SEQUENCE LISTING (26 sequences)

SEQ ID NO: 1 - DNA, 42 bp, Artificial Sequence
  Synthetic primer
cagagacgcagggatgagaagttctctctctctcagctgttg

SEQ ID NO: 2 - DNA, 46 bp, Artificial Sequence
  Synthetic primer
cagagacgcagggatgagaagttctctctctctctctcagctgctg

SEQ ID NO: 3 - DNA, 46 bp, Artificial Sequence
  Synthetic primer
cagagacgcagggatgagaagttctctctctctctctcgagctcgt

SEQ ID NO: 4 - DNA, 46 bp, Artificial Sequence
  Synthetic primer
cagagacgcagggatgagaagttctctctctctctctcgagcttgt

SEQ ID NO: 5 - DNA, 46 bp, Artificial Sequence
  Synthetic primer
cagagacgcagggatgagaagttctctctctctctctcagctgatg

SEQ ID NO: 6 - DNA, 42 bp, Artificial Sequence
  Synthetic primer
cagagacgcagggatgagaagttctctctctctcagctagtg

SEQ ID NO: 7 - DNA, 46 bp, Artificial Sequence
  Synthetic primer
cagagacgcagggatgagaagttctctctctctctctcggtgacgt

SEQ ID NO: 8 - DNA, 66 bp, Artificial Sequence
  Synthetic primer
attataaggcctgctgaaaatgactgaatataaactctctctctacttctcatccctgcgtctctg

SEQ ID NO: 9 - DNA, 68 bp, Artificial Sequence
  Synthetic primer
attataaggcctgctgaaaatgactgaatataaactcctctccaacacttctcatccctgcgtctctg

SEQ ID NO: 10 - DNA, 20 bp, Artificial Sequence
  Synthetic primer
ttggagctggtggcgtaggc

SEQ ID NO: 11 - DNA, 21 bp, Artificial Sequence
  Synthetic primer
gttggagctggtggcgtaggc

SEQ ID NO: 12 - DNA, 21 bp, Artificial Sequence
  Synthetic primer
ggtggcgtaggcaagagtgcc

SEQ ID NO: 13 - DNA, 28 bp, Artificial Sequence
  Synthetic primer
ttgttggatcatattcgtccacaaaatg

SEQ ID NO: 14 - DNA, 26 bp, Artificial Sequence
  Synthetic primer
agctgtatcgtcaaggcactcttgcc

SEQ ID NO: 15 - DNA, 59 bp, Artificial Sequence
  Synthetic primer
ccgcggcccgggcgcggcccggcttctctcttccttctctccttccctcttggagctgt

SEQ ID NO: 16 - DNA, 72 bp, Artificial Sequence
  Synthetic primer
gtggcgtaggcaagagtgccttgacctttttctttctttttctttcttttccgggccgcgcccgggccgcgg

SEQ ID NO: 17 - DNA, 52675 bp, Homo sapiens
caaacagaaagacaaaaaggtgctgggtgagaaaaggagcagagacataaataaaatatccaattttaagggtatagagaggggattcactcaaggaggggagaccatctatctgctttgagaagctgggaaacaaagtcatagggtcaggatggtgcctgactatggatgctctcaaaagctaggcaccaaggatttggactggattcagctggatataagaagttattacagacttggaagcaagattaagtctccgggaaggggagacttaactgggaccagagatcattttcccctataattttaaaggtacttatcatctttaggtactcattaggtacttagcttgtaactctttccactgttcaaatatatacccagtatgcatgtagcctatatggagcaggcacagagtaaatgtttgatgatgataaaagacatgcggaagaaaggttaatttggcaacatcataaaactgaattgagacaaagaaagccaggaggtaggaaagtcaatgaagaagttattccagaaatgtagctgagaaggaaggaatacagaagaggcagatatgggaaaatactcaggaagtataattaaaaggagctgtgactaattttaataaggactgggttaaaaattaagttttcatgtctaaatgttctggaggaccatgatgtcactcaggtaagatggaggaattgagagagggaattcgttagagggaataacatggggaatttggctttggacaggcatttgccatgataacagaatattcatttagaaatggtccaggaaattggtttgatgagaatgaagtgcctgtgaagagagaggactgaagcttgttataatttcattcacttcaggaatatttacagaggacccaaatgtgctaagaactatgaaaacatagaattaaaagaaatgggcctggaataatttacaacctagtaaagcagttatgggaaaacatatttgcaaaaaaggtatacaaagtataatgaaataagtgtccagtaaggataaagtgcagagtaagtgaattaagcagcacccattcatgtgttcaaattcctgccagagtcaaaaggttgtgctgaagtagagtccatgaaagcatcgtagatggctcctcctgctcaagttcccctgctctgcgtcctgctactctggccacaaccgtctggacccagggttgacacacaaacaaacacaataatcttttagccagacataaagaaggccagccaccaatcaggaaaattgtgtcccataaaggcccttcctattgaacagtgaatgacagacatggccagatcttctctcttggaatgctttgaatgttagtcacagagagtgaccactagaagcacagatagcagtagaagctaagactacatgaaaaagcagtggacagatggtgatttatgagaatggcaaaattactagagtcataggcaatggatacttgttaatgaagggatgagcagggccccacagcctgttgctggctcacaagtgcagttgattgctggactgaacagcagctctccgcctgatgatagggttttttaaagtgtccttattgccttaaagtaaatcctcagcatttgcagtgctctgagggtgtcctagcattttataccttttttctaagagcccaggtaacataagggtactcctgttgttctggctttaattctatctgcagaagagggtttcttgtgaaagaaagggtcagtatggtcttttatctgtacagcagataaaaagggtatgtacgtgcacacctttgtacgtggctgccttcccaggacagtctgacagtagagggtagaaacttcagttgtagctgagagcaggcctggaatccccatgcttatactttttatttcctcccccctttcccattgtgatcacaggctacttcagtgtgcttgtccttggagagagcaagggaagggagagccagggagactgttcaagggagccaccaggctcgagaaagaggaacccctgaagacagtagaaagtgcaggtgccaagaatttgaatatctacatcagagtttctcaatgtgcacacagtgaactaccagtttaggatcatttgatttgctaaaaatgaagattactggtctaccttagaccaactgaataaaatatctgggtgaggggcctaggaacttgcatttttggtaggcatggcaggtgattcctaaagcatttacccttgagacctctatgttaaggaaagaaaggtaatgttgcaaggaggtggtgccggcttctaagaaagtacccaggactgaacggcagaaagacctgacataccatatgtataaattgctgtggaagtgaaaaggaaagagaaagtgtctgaggtaaaactggagtgtggggtgcgtggaacaaatggttggatgcagatttgctttacgaatcatgagcctagatgataactgagaccatgtggatggattaggtttctgctaatgccagaatttttataatcagcataaaagtgctatataaagctttcccctcttctatattatagtccttttaagatgtatggaacatcaactataggaagaacatcatattcacagctgtaagaggaaacaagaacttatcatgcacttgatgttgtacaaaataaatctgtgatttatgcttgagtgaccacaaagtagcatacacataagcgcaaattcattcatttaagaattccttgtgtctattatgtacgagataagtatctctgagctgcacggaatgtggcttatcagaaggtgacctaagtttcaaagcagattttgttaagatgaagacagagattgacaggaggtttaagacactctgtctaaagtaaagatttagagtcacagagttcatggattaggatttagaatccacagagggtccacagattcactcattcaacattccataaatatttattgaatgcctttttgtgtcagagactgtcttaggtgctggaaatttagcagtaaatgaaacagaccaaaacccatgccctcatggagcttacattctgatggtagagagacaagaaaacaaaatagatagtgtattattgaaggtgatgagagctctggagaaaaagtaggaaaagagacagatctgggacaagggcgaaattacagtatcaaagatgatctttttagggaagatctccttttaaaaacactttggaacaaagatttaaatgaggtgccagaggggtagcaagtgcatattccctgaggaagacgcctgcctggcattttcaaggaacagccagtaaccaatgtttatctacgtaagtaaggaagggagaacagtaggatgagagttcagagaagagggtaggggatatcaaataatttaaggccatgtaggatttttgagaagaattttgcttttatgtcaagtggaatgagggccactgatgatctgggagtagagtgactatgatccgacatgaagtatactccattttttaactatgtgaacttgtgccaacgttttaacctctaaatctgtttcgtcatttgtaaaacggtaaaaagtattttacctcataaggttgtcgtgatgattaaataagatgatacgataagtgcaaaagatttagcttgtacttaacatagagtaggcacattttctccccttccctgtctttcacttttctcttctgccccttccacctggcgctaggagggggagactggaataaaccttgcagattacagcccgtgtaagagtagaaaggaaaggatgacagttgatgtaaagccttggttaacagacataatagctgggatttaaattcagctttattggtggtttatgatgtggactagaggaatggaactgaaagtctcggaggaggggcgatcctatcaggtacaggcgctgcttttccagccctcaatcctcaagactctcccaagatacatttctaggtagtttatcaacacagactccgggtatgctagcatgtttaattgccccattgtttaatgtcttaactccacgaactttaactgattaatctgtcttctaattaatgtttgaatgactctcctcaggtctaaactaccaaggccatctctacttaaaaacagttgtcttttgtttgtgatttcaggggccctgggtataagcgaagtccctgtttagagaccttgtgatgggttcaaaatatcaagaaagatagcaaaatatcacaagcctcctgacccgagaagattagcgttgaaagggtctgtcgtgtttgtttgggcctggggctaaattcccagcccaagtgctgaggctgataataatcggggcggcgatcagacagccccggtgtgggaaatcgtccgcccggtctccctaagtccccgaagtcgcctcccacttttggtgactgcttgtttatttacatgcagtcaatgatagtaaatggatgcgcgccagtataggccgaccctgagggtggcggggtgctcttcgcagcttctctgtggagaccggtcagcggggcggcgtggccgctcgcggcgtctccctggtggcatccgcacagcccgccgcggtccggtcccgctccgggtcagaattggcggctgcggggacagccttgcggctaggcagggggcgggccgccgcgtgggtccggcagtccctcctcccgccaaggcgccgcccagacccgctctccagccggcccggctcgccaccctagaccgccccagccaccccttcctccgccggcccggcccccgctcctcccccgccggcccggcccggccccctccttctccccgccggcgctcgctgcctccccctcttccctcttcccacaccgccctcagccgctccctctcgtacgcccgtctgaagaagaatcgagcgcggaacgcatcgatagctctgccctctgcggccgcccggccccgaactcatcggtgtgctcggagctcgattttcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggtacggagcggaccacccctcctgggcccctgcccgggtcccgaccctctttgccggcgccgggcggggccggcggcgagtgaatgaattaggggtccccggaggggcgggtggggggcgcgggcgcggggtcggggcgggctgggtgagaggggtctgcaggggggaggcgcgcggacgcggcggcgcggggagtgaggaatgggcggtgcggggctgaggagggtgaggctggaggcggtcgccgctggtgctgcttcctggacggggaaccccttccttcctcctccccgagagccgcggctggaggcttctggggagaaactcgggccgggccggctgcccctcggagcggtggggtgcggtggaggttactcccgcggcgccccggcctcccctccccctctccccgctcccgcacctcttgcctccctttccagcactcggctgcctcggtccagccttccctgctgcatttggcatctctaggacgaaggtataaacttctccctcgagcgcaggctggacggatagtggtccttttccgtgtgtaggggatgtgtgagtaagaggggaggtcacgttttggaagagcataggaaagtgcttagagaccactgtttgaggttattgtgtttggaaaaaaatgcatctgcctccgagttcctgaatgctcccctcccccatgtatgggctgtgacattgctgtggccacaaaggaggaggtggaggtagagatggtggaagaacaggtggccaacaccctacacgtagagcctgtgacctacagtgaaaaggaaaaagttaatcccagatggtctgttttgcttggtcaagttaaacccgaagaaaacccgcagagcagaagcaaggctttttccttgctagttgagtgtagacagcaatagcaaaaatagtacttgaagtttaatttacctgttcttgtcctttcccctatttcttatgtattaccctcatcccctcgtctcttttatactaccctcattttgcagatgtgttctacatctcaagagttattacagtactccaaaacagcacttacatgattttttaaacttacagaggaattgtagcaatccaccagctaaccgcctgaaatagacttaaacatgtgcatctcctttttttttttttttttgagacacagtctcgctctgttgcccaggctggagtgcaatggcgcggtatcggctcactgaaacctccgcctcctgggttcaagcaattctcctgcctcagcctcccgagtagctgggactagtaggtgcacgccaccatgcccagctaatttttgtatttttagtagagacagagtttcatcatgttggtcaggatggtctccatctgctctgttgcccaggctggagtgcagtggcgccgtctcggctcactgcaacctctgcctcctgcattcaagcaattctcctgcctcagcctcccgaataactgggattacaggtgtctgctgccatgcccggctaattttttgtatttttagtagagacgggggtttcaccatgttggtcaggctggtctagaactcctgacctcgtgatctgcccgcctcggcctcccacagtggcatgtgcatcttatagctgaagtctaagccttcttaaatcttgagatccatcaaaacagacaggttttctaattgttatacaatgtatatgttatgtttataatagaaatcattttacaaataagttataaatgggaaaggtctatttgtaattatcagctcagaattaaccataaaactggtgtcactgaagtgactgaggtccaaaatgctgactctgcatgttatagactacagatatcaaatatggttgctaacaatagtttactttgagactgtagccatccacagtatatttgcttttaagagatggtagatggtaattcagttttatgaaaaataaaaatgaattttcttccattacaaaattgttggattcgagtccagtccactccttactagcttttctaactctcggtgagggatcccctcccagcccatgatcttcatttggtaagactcctttggaacccagttctctctagtggatttaaatgtgatttggttttaaaaatctcattcaaggaattttttttttttctggaaacaaccaccgcataaacaagtaaaccggaagatacatgtggctctgaattcatatatatacacaaactctaatccaatgtctgtccacagtatttcctaggctagtaaactttttggccttaacgacccctctaccctctttgtttttttgagagagagagtctcactctgtcacccaggccggaatgcagtggcgcgatctcggcccgctactacctccgactctcaggctcaagcgattctcccgcctcagcttcccgagtagccgggattacaggctcccgccaccgggctaattgtatttttagatacgggatttcaccatgttggccaggctggtctcgacctcctgacctcaggtgatccgcccgcctaagcctcccaaagtgctgggattacaggccaccacacccggcctacactcttaaaaattatcgaaggggccgggcacattggctcttatctgtaatcccagcactttgggagactgaggcgggaggatcgcttgaggccaggagttggagaccagcgtactcaacatagtgagaccttgttataaagaaaaaaaaaatccaggattaaaaaaaatctttgatttgtttgggatttattaatatttaccgtattggaaattaaaacaattttttaaaatgtattcatttaaaaataataagcccattacttggtaacatgaataaaatattttatgaaaaataactattttccaaaacaaaaccaaaacttagaaaagtggtattgtttcacacttcagtaaatctctttaatgatgtggcttaatagaagatatggattcttatatctgcatctgcattcaatctattatgatcacacatctggaaaacttgtgaaagaatgggagttaaaagggtaaaggacatcttaatgttattatgaaaacagttttgacctcttgcacaccagaaaagtcttagtaacctgaggggttcctagaccacattttgagaactgttttaggctatgcaaactggttggggggaggttggggtaggcagagagctagaagatacattttagtgtaattctcctcatctattcctaattgctttggcctacatttgaaataaagcgtggaggcaaacgggataagatacatgtttgtagtggttgttaacttcaccctagacaagcagccaataagtctaggtagagcagagtaaggcggggaactatgccgtgaccgtgtgtgatacaatttttctagcctgtggtgctttttgcggcagggcttaggagtaaggttagtatgttatcatttgggaaaccaaattattattttgggtcttcagtcaattatgatgctgtgtatatttagtgtttatctacaatatatgcacattcattaatttggagctactcatcctataataaatagttgtgcatttactcccatttttttctgcatttctctccttatttataattatgtgttacatgagggaaaggaggtgaaattaaacattcatattatttcaaaaaatttgaaacaactaactaaaaaatatgttttattttctgtatggtgtttgttatacaatctgtcaatattcatgcacctcttgggagacagtgtatgaaaagcaaagagtaacagtcacatggattactgattactgagatatattcacttgcatctttttttttttttgagacggagtggctctgtcgcccaggctggagtgcagtggcgtgatctcggctcactgcaagctccgcctcctgggttcacgccattcttctgcctcagcctcccaagtagctgggactacaggcgcccgccaccacgcccggctaatttttttatatttttagtagagacggggtttcaccgggttagccaggatggtcttgatctcctgacctcgtgatccaccctcctcggcctcccaaagtgctaggattataggcgtgagccaccgtgcccggctcacttgcatctcttaacagctgttttcttactaaaaacagtgtttatctctaatctttttgtttgtttgtttgttttgagatggagtcttactccgtcacccaatctggagtgcagtggcgtgatctgggctcactgcaacctctgcctcccgggttcaagtgattctccttcctcagcctccccagtagctaggactacaggagagcgccaccacgcctgattaatttttgtatttttagtagagagagggtttcaccatattggccaggctggtcttgaactcctggcctcaggtgatccacccgccttggcctctgaaagtgctgggattacaggcatgagccgccgcacccggctttctaatctttatctttttttgtgcagcggtgatacaggattatgtattgtactgaacagttaattcggagttctcttggtttttagctttattttccccagagatttttttttttttttttttttttgagacggagtcttgctctatcgccaggctggagtgcagtggcgccatctcggctcattgcaacctcggactcctattttccccagagatatttcacacattaaaatgtcgtcaaatattgttcttctttgcctcagtgtttaaatttttatttccccatgacacaatccagctttatttgacactcattctctcaactctcatctgattcttactgttaatatttatccaagagaactactgccatgatgctttaaaagtttttctgtagctgttgcatattgacttctaacacttagaggtgggggtccactaggaaaactgtaacaataagagtggagatagctgtcagcaacttttgtgagggtgtgctacagggtgtagagcactgtgaagtctctacatgagtgaagtcatgatatgatcctttgagagcctttagccgccgcagaacagcagtctggctatttagatagaacaacttgattttaagataaaagaactgtctatgtagcatttatgcatttttcttaagcgtcgatggaggagtttgtaaatgaagtacagttcattacgatacacgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagtacttattacaagataattatgctgaaagttaagttatctgaaatgtaccttgggtttcaagttatatgtaaccattaatatgggaactttactttccttgggagtatgtcagggtccatgatgttcactctctgtgcattttgattggaagtgtatttcagagtttcgtgagagggtagaaatttgtatcctatctggacctaaaagacaatctttttattgtaacttttatttttatgggtttcttggtattgtgacatcatatgtaaaggttagatttaattgtactagtgaaatataattgtttgatggttgatttttttaaacttcatcagcagtattttcctatcttcttctcaacattagagaacctacaactaccggataaattttacaaaatgaattatttgcctaaggtgtggtttatataaaggtactattaccaactttacctttgctttgttgtcatttttaaatttactcaaggaaatactaggatttaaaaaaaaattccttgagtaaatttaaattgttatcatgtttttgaggattattttcagatttttttagtttaatgaaaatttaccaaagtaaagaccagcagcagaatgataagtaaagacctgtaagacaccttgaaggtcatggagtagaacttccatcccaagcagatgaggatttatttaatctcaaagacctccaggaggggacattccccaactgtccttgttaactcattttcagaacatatttattagcatattttacatgtaatttggatcttcatgttaaatttaacatcagtggagatggaaaataagcatatcgccttgtctttgaaatagccctatattgttagattgtttcttaggcttctttaccctgggttaagcagtcctaatactttagcatttattctacatctagtgtactaatttaaaaaaatcagttctgaaaaatttctaagaactttcttcaagttccaagctgtgaaatctagaacaggtcaaagtgccttattaacgtactgtactgtgtagtgtcttgaagagacactttgcgctgaggcaagttctgagggcattgggtggccttgggaagatatttatgcagtttagaacctggagaattgattagataactaatcataaggaaacgtcacatatttttggtactataaaaaagtggagaaataatgcctatttgcaaagatttgatttaaacatagaaacaactttatttggcttccaattttaagaatttacagcagtaaaggggaacagtctaattgaagtagactgcctatgcaatagtctctgtatatttacttttgacaagttaattcaatgtgtactatagttttgtttctttgaagaggtttgaatagtgcacccattttaatctgtattgcaaattcagggttacttggcagactctactatttaaatcagatgtaaaaggaagttttaatataattcactttatgcctgaaagttttcctgggattttggaaggtgattttactggaaatgctgtctgtcttccctgaaaatctgagaaattccattacactttgtttccaatcagaggtcatgagtgctatatgagtatatacagcatgacgtcatgaatgtgataaagtgggttaggaaaccttttgctaatgattgttaaaatgcaatataaatgttgaagaaataaagctaacagttaagcctttatttgggcggaaggctgaaaaagtttataaacttaaacctataactctgcttatgatttctgccaaaccagaagacttgactctgggaagcattggttacctgtgaactttgaaactgacggtccctgacgtagtttagtcacctgggaaaaggtatctgagattatctcttatctcccaagttacagtgagtctctgagggaactgacacattacattaagttcttggtgtagttaaactgtaagaaaggcaggagaacttagtagttaaatagttggttaaatggaaatgctgactccatgttattgtaaaaagttaaaaatttaggaggatatggggatttcactgccattgcaggttttgattggtatttaccaatccgtgtgggtcagagagaaaattagaaaggatatgactgcacattttggaattattagcagtttttctacatttaaaatggaaataaattttttaaaaatttaaatcaagtaatactgtattttttggtgatttagatttttcaaaatttacactaagagatagtaaggagggtggctattgtttctttcaataatgtctctgagaggttgtaactcatctaaggatacgtagctaataagtggtaggatttcaatttaaattctctgagaccaagttaagtagaatttgcactgtactcttgtataactttttaaaactgaaaattagctatctttcaaattaagaaaatatttactaatggagactaattcagatttgtaagtataccaaaatttgaacttagcctgctatctaatggcaacttagtggcagaggtatgatgtaaaatcattcaggtatgacacatagatggagtatgtttgtattcgaggctgtgcacataatcacctttacttgtattgtgaagtatatattgttatcttttatgaagcccactaaagagataatgaaatacctcgttattagggcaagattattgaaaactcaaaatagcccccaaacacaatacttggctagaaatatatacctttatagttcagagatcatttattatcaaaaccctgaagttttttttctaaggtaaaatttggtggaagaggaaaagtctcgttttaaaaaaatgtaggtagttacagagatcagaatgattagttgatcacttaccaaatatatattaagtatctactgtatataatatgctagtaagaataaatatagcaggaagtattttttcccaggctctaattgtttgacatcagcatgcttttattgtggcacttataattcagttcaagtattatgcccctctttgatggaacagtttcctattcagtaaggaagaccagattaatcattggattggtttgtttcatctttagtgttctgagctgtagagtatttatttaccaaggtttattttaatttttattttatttttatttttccatgttcattgtagaattcattttacctacgaatgaagtatgtagattatagagagaaaatttgtaaaattaaactgatactgaagactggtataagaaaagccttatgtaatttgtaagctgctattcttctgagtttatacatatatctttagtaatcaatgagggatggttgggtgactgccctccaggggacatttggcaacatctggagatgtttttggttgccacaacttggggagagagtactgctactggcatctattgagtagatgctattactttaaatggcaaagctgcagttacctttgcaccaacctaatattaaacttcctgcagtgcacgggaaagcccccacaacagggttatctgaccccaaacctcaatggtgttaagatccaaaccttgatatgttaacctgtagctttaaacatcctttaaattgtcaaattcatgtccctgacataaggtttatgttagattttcaagtataacaaagatttaaactttaacttttgtacgttaatgatatgttagcttactccagtcttctattaaaacattctgtttttaaaatcagagacacacagcaattttataaatcatttctcttcaaggctgtgaagctctccccacttttgtgagtgccctctactggtcaaattatttgctttataacaagtaacagtgaaatcctaagtttgtgtagtttcgctgtttaaattatgggtggcatcaatttataaatatattcgttttatttaaaagtcttatatgattgatttcgtatcatttttgctctctgctaatattaatataaagattactgtctgtattagttaggcctaactaagtaggtgagtatagtgaactaagaaaggaaacgaggcagtatataagaaaatagggtggttcagttgttaacacttactgagcttactttgttgaagggactaaaaggcagcagtgtggctctctgagcttctttgcatgcactcaggagctgcttaatggagtccaaggcttggtggtgtgttacaggggatgataggagggtcctattcagaagtggcaaattgtgaaagtgcacattttgtagagttttataggactgtagaatagttgtgagcacctgatttttagaataaacagaaaactcaggtactgtatttaggtcaaattaagaataagtatttattaagacctgaatataaaactttactggtcatggtttttttctaccttgggtttttataaatccaaagatttaaaaacatacaaatggaagttggtaatggaattaagtgaaaggaaaaaatgattttatggtttggaatctcctaagattctggttttaacaatacaactaattccttaatcctagaaatgttcttcactgcccactttgtaccatgcagtcttcctgtgggctagagatacactgaggcgcaaaacagaccagattcctgccttcatggagcttattagttttaggtatctctagatttcttgtaatacctattacaatgcctgcacatcagttcattcatgtgggttcaacgtagtactcagtacatggcaaattcaagttttacttttcggaacttcatggatttttttcctcagaatatcttttatccataattggttgaatctgtagatgcagtacccatggatatggatggcccactttattttgaagagcagtgtttctaggcaatcatgctaattatatatgacttaatttagaggctttatacttaagagcattacatttctggcgtctcttaaccattattatttcataatgtgtaggttatggaacagttaaattattgggatcttaatatagaaattagtagaaataagccagatatggtggctcatgcctgtaatcttagcactttgggaggctgaggctattcgctgtactattttttactacttttctataggtttgaaattttttcaaaataaaacattgaaaaaagtaaggtaggtagtgtgtccctccttaatcctttcaaatattttattttcactatttctattaatttttttttttgtttttgagatggagtctcgctctgttgcccaggctggagtgcagtggcgcgatcttggctcactgcagcctccacctcctgggttccagccattctcctgcctcagcctcctgggtagctggtattacaggcatgcaccaccacacccaattactttttgtatttttagtagagacggggtttcaccatgttggccaggctagtctcgaactcctgacctcgtgatctgcccgcctcagcagtgtcactgcttctagaccgttttcaaggcacagagcttagaaatgcatgttactaagaaatcaagagttaactatttttcaccttctttctcccgcagtgagaaccctggttctaccctgtttctccttgtgtaaattttaatgctaaactatacacttgtgaaataaaaatgataatgtcattcttaaattatggatcttgcagtgttatctaagtaacatagattgagtgatttaactttaggtttccttatttgtggaatttggataaatatttttcacccttgagaaaagtgagactcctttctcatcatcagagtatccttaaaccattaaggcaaacatttgggaaaaaactgagctatctggctgcataaaaattaagttttctttaacaaagatagaagacaaatgaaaacctagaaaaaccatttggttcaagtaacaggaagctatcttatatatgaattagagaaaagcaaacacacaaatagaaaaaaagggatggggggtactaaagatataaatagcttgtctaccaaaaaagaaataaaataaataacatgaacatataaaaagacacttacttcatgaatgtgatgcaagttcaaacaataaataacatttctgtactttcatattggctaaggttaaaatgataactgctaggaagggtatggagaagtgtgcgccttgcactgtagtgggagtatagaccctcagactttatggaggtcagtctggaaatatgtttcaaaatgtaaactacatgtcctttgaccaggtaattcaacttcttgaaatttatccaaggatttaattggataaatgtttaagatgtatatataagaatgtttactgcagtgttgtttatgattttaaaaaaatggaaatcatcttcatgtctaccaatagagaatgggtgaataaattatggtatgtccatatatacaaattacatagttgttggaaatattaggtagatttagatatactgatgttcaaaaatgtccattatgtaagtgaagctgggtcacagcaccttgtgttgagtatgatttcatctagaaacaaaattactccctcatcctttgttgtgttttagttttttaaaataagcttataccattgggctgggggaaaagtaaatactcgttttggagagagaaaagggcactaaagtttcagataccgttagattatttcatgcttatttttcaagcctcaataaattacataattcacatgtagtcttggattaaggaaattgctattaaggctaaataaataatatgagaggtatataatataaaatatgaacattatattggcattaagattggatccacggtcattccagcctctcattcttacctggacttcaagtgatcacttgtgggcaaatgccatctgacttgaacaggttacacatgtatgctcattatatcgttattttcaaaatttgtcatataaattttccttgagttcattcagatttttgaactagttttttctcttgggagtagtacacacttaattctctctagtactaagctaatgttcaccattcttataattttaagtatccagcatttagtaaagaagtctttgttttctttatccttacttttagtgaatgtcttagtttttaattgaaaattctgccatgaaaataagctctttaacatcttcactccctaatcaaaacagaaatccttcatagccttcagttgtagctatccttccctgtgatttgtccagctccattatatttattttgaaatatggtgaccagttttgcaaaattatttcaactgtaggtgcccagtgattttgtaaggagaagatactgtttctgaacagttctcagtagccagtggcctgcccctactttttggcctgcgtgtagtatataaaataatgcagttaactttttatagcacttttcattttataaagagattttcatggtctttaatattaatctatgtataaagtcctgtatgcagttttacctactttcacagctgaaggaacaatagcttagagaagatgtgagataaagtagtttgcccaagcccatagcacaaataagtgaagttcttcggctgtccatggatcgaagactcccaagtctatctctagcctggacttctgtcctgagcaccagacatgtatgtatatcaagatgcctgcaggtcatatccaccaggacaacccatgagtacagggaattcaacatgcccaatatcactcatcttttccttcgccctcccctttgtactcatcccctgtcggtaagctctgttattttaaaaaattgaaatgtattcacatagcatacaatttacacttttcaagtgtacatggtttttagtatattcacaagggttgtgcagtcattactactaattccagaatgttattatcaccccaaaagtcccacatccattagcagccactccccaatcccttctcccaccagcctctaaaaactgctaatttttccatctctgtggatttgtccactctgattatttcatataaagagaatcgtacagacgtggccttttgtgtctggcatcctccacacaggatgatattttcagagttcgtctatgtttttgcttgttgatcattccttcattcctttttctggctgaataatactctgttatatggatataccttattttgtttatctgttcatttgatgggcatttgagtgatttcctctttttggcaattttgaataatgccactataaacatttatgtacacgtttttgtgtgaccatatgttttcacttctctcgggtgtatatctaaggtacagttgctgggttatatggtagctctgtctttgactttttgaggaactgccaagtggttttggtagtgattgtactgtttacattcctaccaacaattttacctaagtatttctcaaatctatttaatcttttcggtccatactgctgttgctgccttagttcagattttgtcatttcttgtaataattcgtagctcatctcccagtctctgctcccctctctccctccctcccccttcttctctctcttatttccacccatttttaacatttatagaagtcaaaagtctagttcagaaagcagaaaccatactagatatttcagcacagagaactaattaggtgttggaagactgaaaggcaaaaaaacactgaagtaacacagtaacatcaagaatgggcactactcctaagattcagggaatgctgggaagatttggggtttatcagaactggaagctcagaggaggggccccttgtcgctgaggcttaatccctgcagaggtgcctttggctgctactggtgaatctgagtgggtatgatgagtcagtgtctgggaagggccaaaacattttgtccctttctataatttgtcatgataatgctagtaatgaatctgatctcccttcctattttaaaaaccttttagtgattttgtataggatgaagtttaaaactccttacttaatatacacatgaccctccgtaagctggcccctgcttgattgtccagtttcacttcttggtgcttattctaaggcctctaagccttagagatcctctaagcctttgagatccccaaaccctggactgcggactggtacccacctgtgtggcctgtgaggaactgggctgcacagccggaaggaggtgagcattacttgccttagctcctgtcagatcggcagcattagattctaataggagcgtgaaccgtgttgtgaactgcccatgcaaggatctaggttgcatactccttaggagaatctaactaatgcttgatggtctgaggtgaaacagtttcatcctgaaatcacccccaactcggtccttggaaaaattgtcttccacgaaactggtccctgatgccggaaaagttggggaccgctgttctaagctaaagttatatggagctccttggttctgtgtcctcaacatgctgttctatgttttttacattctgtttgctccttcctgcttggaatgtccttcccctccccgtctttcttaatgcatacaaagttgatctctcctgtgtgccaccattgtacttcgtcttgcatatggtgttacattcattttattttaattatttatttacgttcatgtctcttccactcaccttagttgcttgaggtcagaaactatataatgtgtgacacggaatgtgacacctagattttcaataagtgtttctatgatacaagggagactgatgtgggtagatgggaatgaactcatcaacctctgtttacataccctaaattccctgtttcttccctattataattctgacagtctacaaccgtctttgatggcttataaacggaaagtgcggaacacatcattctacagtgaatttaaataacctttcggaagagtaacgtaaagtacttgagcattaattgagtaaaagtttctcatcttttcctacaggtgttattaagcagtatgtaaaaagtccttacaatacttaatacattaagaaaacatacaatttcaagaggaaatccccgagtaatacattattgacattttcagcagttctagttatattgagaagagcatctcatggaattggcagaatgaagatggagattaaatgagatgatgtttgtaatatgcttatgacagtatctggcatataagtaagggctcagtaaatgttgactgctgtaattactattaatagtaatatgattacctttagtaaaagttattagtttctttaggttttttgtttactacaatatagtaaacaaaatctatacttggaatgtatatattgttttgttttgatacatggaatatgtctctgtgtcagagtcactgcctgagttggaaaacccatactcgagtatgttaaaaggtgaacacactgaataatttagttattaattataatggaaaaatgacaaacttgatgttctggttaatgaggttatcttatcttgaatgagttagcttttaaattcctcaaaataaaggcatttaataaaccaggaaacacttcattaaaaaaattatgcaagtcagtgtaaaagaagattaaaattccacatgggcaaaggacacacgttggcgataaatatgcagataagaaaaaaaacctatataacattattactcctcaaagaaattggtatgaaaacaataaaaatgtgtagcttatcaaaccaacaaaaatttaaaaatatgaaatccattttaagtaatgataaaatgggtgcactcttagtgctttatagaatagtagtataatgaacctcatgtgtgtaccaaccagctctttcatatcttaacatttagcaacatttgatttagctctttcttttttccaagatagaaaagttaatattgttgaagactcctgcattcttttccctagtcttattttcttccctcccataaatgtgttaaaatctctgtgtgtattgttttggttgtatttttacataaaactttacatattatataaaatttaattgaaggtaaaatttattaaattattcttaatatatattgtaatttaaaaattaacagcttcattgtcttgataaaatttatggtatcttaaacatgtgcttgtttttctaagagaacattgaaacatagattttaaaacaaattgttgaaagattaaaaaatctgcctttgcacactgttacattgaaagtggggcatttgtcgtgaacattcatttcaaatatgtagtatcttcagaatatttgagaaggatttgtattatataattgaaaaatctgttaaattgtatttatgttaactgcttaattctaataaaatttccattcattttttagtatctgcatatatttacatcaaatggattcattcacttatttaagaggcagtactaattacctatagcgttcaagactgttaggtagagggtgtgtagtggtgagtacaacaggcgtgagccctaccaacacggagtttaaagcctagtagaggatatagacttaaacaatttcacaagtaaatacataattacaaattataatacatgctatgaaggaaacataggaggtaccagagaaggaagagtgctttgcatttttatttttaagaccgaagagtgctattggaggactttgagcaagtgaatgacatgatctaacctaccttcgttcattcattcattcattcattttcttccttcctggctcaagcagtcctcccacctgagctccccaaatagctgggactacaggtacacactaccacacctaatttttttttgtattttttgtatttttgatgggattttaccatgttggccaggctggtcttgaactcttgacctcaggtgatccacctgtctcggcctcccaaggtgttgggattataggtgcctagcccatggtgcctagccctaacctacatttataaactatcacttgctgctgtgtggagactatattgtgagattaacagcagggatacctgctaggaagcaattgctgcagattgcctgagacaaaatagttatcatggactagggggatggtggtggtggtggtggtaggtggttggatgtaggatatattttgaagataggtaaatggtgcaagattatgggtcagttttaaatgcttaagtaaattttctttgtaagacattttaggatgccatgttaagaatctctttataactgtcatttaaaaaaaaaccacatattttcttagcataatttcccatagtaacattactatgtcaaaggctatgaacatttgaatgactttagataaatactgtaattgctttccaaaaatattgtgcttattatgtcaccagaaatgtttgaattctgtctacaattcagtcttgccagtatagtacatttcatttagaaaaattttttactatgtagatggaaaaaataatattttagctgggagtggggggactatggggaataactttccttcatttaatattttattgtgagttagtttaagttactttattttatcgtagtttcctaaggctacaaattagtaaccttggtaacttatgtacctaatttaaaagtttacttttttgaaaggctggaaatactaattaaaaacgtaacaccttcatccttgtctttgctccattattaactagtttcattacagaatctctgtgttttaaaatcagatgggttttcataaccagtactttctcagagtggtaaatttaaaaaaatatataaagagaataaataatatttgttgagaatacttcaaataatgtgaagagttattaacttacagcaggagttggcaaacttttctataaagggccatatgggtctttgtcacaaagtcttgggtttttgtttttgtttttttaaacagctatttaactattcctagctaatgggcaatacaaaaacagtgggcaagatttggcctgtgggcagtagcttgctgaaaccttatttagactctaaattttttgaaagagtctacattgatgcatatttttttttcttcctccaaatacagttgacccttgaacaacatgcgtttgagtgaccatgggtccacttgtgatacacgtttttttcccaaccaaatgcagatatggagggctgacttttcatatacctggatgttcctgggccaactgtaggactagaggctgggggggtcttggaaccaatgccgtgtgtataccagggatgactgtttcttatggcctgacctgaagttggaacagaatctttattaatatataatttttgttgcgtttgttttctctttatatttatccattctttttagatcgtatttcatttaacactttttcttctttagtttttaccaagttgcactgaaaatagctcagtgactaattgcacttctaagagtgaggaccctagttaaaattaactctaaaaatactgaatttttaacctaaaccttttatttctaatcaacagtattatttatgagtaggttatagattactttgaaacggaatgtgtctcagaactttgctatcgatatttttaaggtctggtagggaaaagataataggaatgagatttatcagtgaataggggactgctttcccagtttctcggtcgcactggtgtattcaccatggaagcatcttatgaaatatgtacataaactactaatatcccacattacaggttgactattctttatctgaaatgcttaggacctagaagtatttttggattttggtttttcagagtagggatactcagcctacattggtaagtaaagaatgtgaggtgacaggctgggcgcgatggttgacgcctgtaatcccagcactttgggaggccgaggcggatcacctgaggtcaggagttgaagaccagcctggccaatctgtactaaaaatacaaaaattagctggacacagtggcacgtgccagtagtcccagctactcaggaggctgaggtaggagaatcgcttgaacctgggaggcggaggttgcagtgactcgagatcgtgtcactgccctccagcctaggcaacagagcaagactccatctcaaaaaaaaaaaaaaaaaaaaaaaaaaagaatgtgaggtggcagcaataggtaggaagagtctttggtcagctttacatgctctgtagccatgcctgggtaatgggttgactctaagactctgtgctttgctcccacctcctgctttttcattactctttagaatggtttttaatttgtgatctataggagttctttcaagtatttaataagagaataggctaaattaagtaaatgtcaactgaatgctcaaatctctactaaagagcctcttatttagaaaataaatatccatcttttttttctgactggtgagataattaatttttattacagatggtttggaaaataccatatgctttaaaagataagcacaaaattatagtctaatatgtaggttttcatactttaaaaaattgaaaaccaaagaaaaacatttaacatagcatctagtacaaagaaaagagataagcaagagataaatgtcttttttgggacagagttttgctgttgttgcccaggctggagtgcaatggcacaatctcagctcaccgtaacctccacctcccgggttcaagtgattctcctgcctcagcctcccgagtagctgggattacagtcatgcaccaccaggcccaggtaattttgtatgtttagtagagatggggtttctccgtgttggtcaggctgatctcaaactcccgacctcaggtgatctgcccaccttggcctcccaaagtgctgggattacagacatgagccatcgcacccggccaagataaatgtcttttaaattatctccattaaagacataacctttataacattttgatgtatatattaccagtttttaaacacatagtagatttgtataaatacataaacacatattattgtgatcatgctgcacttagacatctttatattctccttatactgtaaacattttgaaatactttactaacaacatttgtaatgaccattctttctctctttctccctctgatagaatggtctacagagtaattcataaactaaacatactttagaggctgggcgcagtggctcatgcctgtaatcccagcactttgagaggctgaggcgtgcagatcacgaggtcaggagttagagaccagcctgactaacatggtgaaaccccatctctactaaaaaaacagtacaaaaattagccgggcgtggtggcgtgcacctagaatcccagctactcaagaggctgaggcaggagaatcactcgagcccaggaggcagaggttgtagtgagccgagattgcaccacagcactccagcctgggcgacagagcgagactccatctcaaaaaaaaaaaaaaaagatacattaatactatagcctacatgtggaacattaagaaaataattgcttttatgtttatgctttatacctgttgttagccctgcttcttatttcatgatttcatggcttcacattgtaacatccctttaccatattttttgaggactgttttggcagaatgtgtgaaatcttgagcagaagtattacccaaaagtcagaagaaaatcagatttttatttcaagattctgttaaagttacccactcccttcttttacttaatcttatagttgcagttctctctctttttagaaaagaaaaaagaggcccctcaggatttgcagatgaaacaatattgctctttagagatatccatctggctgttagattatttttccacagttttcagaagtggatgaggccattagaatcttgagtattgcccatttccttatgtgtgcctttgactatagataaaatagatgcatgacaattatttataagttgattgatttttcttgtcatttaaatcatcttgaataatagagttggtagagctatcccatttttgaaattattttgttttgtcaataactttttgttaccagcatgtacacttgcattgttgactctccatataatacctttaaaaaatttttttttgtggtaaaatatgcataacataaagtttaccatggtagttttctttcatttgttttgtttttgtttttttgagacggagccttgctctgttgccaggctggagtgcagtggagcgatcttggctcactgcaacctccgcctcccgggttcaagcaattcccctgcctcagcctcctgagtagctgggactacaggcgcccgccaccacgcccggctaatattttgtattttaatagagatggggtttcaccatgttggccaggatgttcttgatctcctgacctcatgatccgcccacctcggcctcccaaagtgttgggattgcaagtgtgagccaccgcgcctagaccatggtagttaattttaagtgttcaattcagtgaccttaagtgtgttcataatgttgtgcaaccatcaccatgttgtctaaccattagcactatctgttttgagaacttttttttatcatcccaaattagaattctgtacctgtcaaatagtccccagtaatcctccctcccccagcccctggtaatctgtagtctacttttcgtctttttgaatttgcctattttaggttcctcatataagtggaattatgtggtatttgtccttttgtgttggcttacttcatttagcataatgttttcaaggttcatctgtgttgtagcatgtatatacaggttgaagcatccgttatccaaaatggttgtgaccagaagtggtttggatttcagattttttttttggattttggaatattcatagatacttaactggttcagcatccctcgtccaaaaatccaaaatcagatggagctcagtggctcatgcttgtaatcccaacacgttgggtggccaaggcaggaggatcgcttgagcccaggagttcaaccagcctgagcaacacaagaccctatctctccaaaaaaaaaaaaaaaaaaaaaaagatgaaagaaaaaaaaatccaaaatcaaatgctccagtgagcatttccttttagcatcatgtcaggctctaaaagttacaggttttggagcattttggatttcagatttttggattaacctgcattaatgctcaacctatatgaaattttattcctttttatggctgaataatgttccactgtatgtatatactacattttgtttatccattcatctgttaacagacacttaagttatttccacattttgggtattataaatagtgctgctgcgaacattggtgtacatgtatctgtttgagtccctgtttttagttattttggttatatacctaggaatggaattgctgatcatatggtaattctgtgtttaactttttgaggaactaccactgttttccacaatggcatcaccattttacattcccaccagcaatgcacaaagatttcagtgtctgtatccttgctaacacttattttccattttttgagtttttttgttttgtttttttaataatagccaatcctaatgggtatgtggtagcatctcatggttttgattttattttcctgactattgatgatgttgagcatcttttcaggtgcttagtggccatttgtccgtcatctttggagcaggaacaatgtcttttcaagtcctttgcccatttttaaattgaattttttgttgttgagttgtatataacaccttttttgaagtaaaaggtgcactgtaataatccagactgtgtttctcccttctcaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattataggtgggtttaaattgaatataataagctgacattaaggagtaattatagtttttattttttgagtctttgctaatgccatgcatataatatttaataaaaatttttaaataatgtttatgaggtaggtaatatccctgttttataaatgaagttcttgggggattagagcagtggagtaacttgctccagactgcatcggtagtggtggtgctgggattgaaacctaggcctgtttgactccacagccttctgtactcttgactattctacaaaagcaagactttaaactttttagatacatcattaaaaaagaaaaccataaaaaagaatatgaaaagatgatttgagatggtgtcactttaacagtcttaaaagcaatcgtgtgtatagcatagaattgcttggattggataaacagtggcattatatattttaaaaaataaaagttttgaaagattgaagaatttgggcattacagttctcttaaatctgacaaagctgcataaaactattaaaataatcattattatactattttatattctatttctttgagggtttagttttccaaaaactacatattaagcaaatgaatcactcagtggctatgtcatataataacgagttagcctagttataagaagtttaacattttatttaagaacattgttacagcatgtttactgtatagtctagtaatagaggaaaagacatttgggtgggtggtagtggtagtatttttatagaggagttaccaaatttcagctctattatccaagtttacccagctaatggtgttcggaaccgggaatttgagccaattctgactctgttgtctgctctgctccttcttttgtgctgtgtctttgaaagtcacctaaaattgtgagggaatgtaatttcaccccaaatttagagtttatgcacttgttatattgaaaatgattaacatgtagaagggcttttaatggaataagtggtgtagtaacttcagtgttgcctacctagaaatcaaaatctttctagttgtccactttgttttttgaaaaagtaatatgaaaattatgttaatgctttaattcaggtttttgtaaaatattttttatctttacacatttaacatacgtttctaaaattatagtctgttatatagcactttgggtctagaatttttcagtagtttctgttttactattatgatctacctgcatattaacctattaggttatagttttactatacttctaggtatttgatcttttgagagagatacaaggtttctgtttaaaaaggtaaagaaacaaaataactagtagaagaaggaaggaaaatttggtgtagtggaaactaggaattacattgttttctttcagccaaattttatgacaaaagttgtggacaggttttgaaagatatttgtgttactaatgactgtgctataacttttttttctttcccagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacaggtaagtaacactgaaataaatacagatctgttttctgcaaaatcataactgttatgtcatttaatatatcagtttttctctcaattatgctatactaggaaataaaacaatatttagtaaatgtttttgtctcttgagagggcattgcttcttaatccagtgtccatggtactgcttttggctttggtttctttctacattgaaaatttctcttcaattctgagcacatgttaacatttagaattcaagaggtggggatttttttttcccatggttacatatatatatatatatatatatatatatatatatatatatatatatataaagaacagggcaacaaatttttgcgttttctatttcggtagtacttttaaaccattatgtcatgtttctaggttaaacgttgttgtatttgaagaattttactttggcagaatttttttgaggatgtgtttatttctggagaaaggtctcattaaagaaagacaatacccagaaagccaacagaaattctgttactcatttaatgcatttttctgacaaaaattattgccagagagaacctgaattttgtttcaaaaatcatctttgttttaaaaatgactttttcttcaggtaaaataaaataatttcagttgctattatttaacctgtttgtatgaagagtttaacatataggaaatgaatacataaagataggaaggaattaattgttatatgtagtcatatgtctcttaatgacagggatactttctaagaaatacattgttaggtgattttgtcattgtgcaaacatcatagaatatacttacacaaaccttggtagtataacctactatacacctgggatatgtagtatagtctcttgccccagggatacaaacctgtacagtatgtaactgtactaatgactataaggcaattgttaacacaatggtaagttttgtgtgtctaaacctacacttgggctaccctaagtttatatatttttttaaatttctgttcaataataaattaaccttactttactgtaactttttaaactttttaatttttcctaacattttgacttttgtaatacagcttaaaacacacattatacagctatacaaatttttctttccttatatctttattctgtaagcttttttccatatttaaaattttttgtttgtttttacttattaaacttttttgttaaaaactaagacatgcatgcacattaacctaggcctacacagggtcaggaccatcaatatcattgtcttccacttccacatcttgtcccactggaagatcttcaggggcagtaacacacgtggagctgtcatctcctataataacattgccttcttttggaatacctcctgaaggacctatccaaggctgtttatagttaacttttttttttttttttttttttttttagtaaataggaggagtacactataaaataacaatataggtgctataccattatacaactgacagtgcagtaggtttgtttacaccagcatcaccacaaacacgtgagcaatgtgtcgtactacagtgttaggatggctataacatcactaagcaataggaacttttaaactccattataatcttatgggaccactatcacatatgcaatctcctgtggaccaaaatgtcattatgtggtacatgactgtactaagaaattgatccatctatattccatcaatttgtttagggctttttctggttacatttacctgtgagcccagaaaaccagttttgtagaaattaacttctgtaatgctaggagttaaaaaaaattgctgaacaacttttacattgttaaacatttaaaaacaagcgttctagaagtttatcaaatttcataaaggtgcaaaaatgtaaatgtaaatcattatccagctaatatatatgttgtatttccctagtaggagagcatatgtacctcttcctagttatacaaatttgatatatagtaaagaaacagtaaattctacttcaagtcattttgggaggattaaaaactgaatttctctagtttgaccattgtacagatttatctggcaattttactaaaacctgatttataggttaaacttggtgtatatcatatatcactttactttagaggaattaagatttcacataaatccatttccaggttccaaagaccaggaagaggcttggtttttgtttttctttttactgtctttacagtctccttgacttttcttaggagagaaggtactgagaaaacatgattctaatatttattattttttcttccaacattttcttatgaaacattttcaaatacaaaattgagttttatttaaaacatttgcaaatatactacctagattctaccattgttgttttatatttgctttacttacaacttttaaaagatgctttttataccactgaacattttagcttacatttcacaaagaaaagaaaaaatttaagagactttgcataatgttttaaggggttgcagtaaagaagtgcttcttatattttcttatgcatacaaatcagctgggcttattaaaatccagattctaattcagaaggtttaggtggggaccgagtctgcatttctaacaaactcctaggtggtatttttcttggtacttggaccatactttgagtagaaaagcagtagaggacataaaaagagtcttgttagtcccactttgttgctgtccacttctcatttgataatatcctaaaatagctgtgtctcctttttggtggttgtatgattactacctcagaagtactaattgattcttgctatttgaccttaatactttaatataacacagcattcatatttgatcagaaaactatctggcttccttttataagagatttttaggttttatacagttttgtggccttgggtttttttgtttgatttgtttttttgaaggtatataatatgtaagtagataaacaaatttgatttgtagacatttttatgtggatcatctaattaaaaatggagggatacagtatgaaagaatacttgtacttcttaacagagcactcaacctttcttttacatcctgtttcactgatgttattatgtaatttatgttgctaaactataaattagatatttaatttctgttctttgatttccttttattattaaatggacttgttgatttgcctagaaattaatttgcctttcaaaagtcttattaatcttcctccgttgaaattaatttgatatttgcatgcttctggaagactttaaagagctattccgagtaactgtagagattataaaatgaaatatgggaattttaataaattttacatctccagttactggtgaaaatgtcaagtcctcctttctgcagagtattttgttactcatctgttattcagcttatttatttatttatttatttatttatttttctttctttcttgttttttttttttgagacggagtcttgctttgtcgcccaggctggagtacagtggtgggatcttggctcactgcaggctccgcctcccgggttcacaccattcttctgcctcagcctcccaagtagctgggactacaggcacccgccaccatgccttgctaaatttttgtatttttagtagagacgggtttcactgtgttagccaggatggtctcgatctcttgacctcgtgatccacctgcctcggcctcccaaagtgctgggattacaggcatgagccaccgcgcctggcccttatttgttttttaaacaaaattagtgtgcatatccttgttgtattttatcggcaagttgttttatgccctaacttttggggtcttgatcatgagcctaaaacacgtaaacacccaaaaagaattatattccggttaaaggaacaaaacattcatttagaagttctcatccatgtaaatcagaggctggcaaatattttctgtaaagggccaagatagtaaatgttttaggctttgagggccacaagtggtatctgttgcatttttttttaattatgaccctttaaaatgcaaaaatcgttgttagcttgtgcatagtataaaaataggctggccgcatgctgtggctcatgcctgtaatcccagaaatgaggtgggaagccgaggtgggcacaccacctgaggtcaggagttcgaggccagcctggccaacgtggttgaaaccccgtctctactaaaaatacaaaacttagccaggcgtggtggcgggtgcctgttatcctggctactcaaggggctgaggcagtagaattgcttgaacctgagaggcagaggctgtagtgagcccagatcaagccagtgcacaccagcctggacgaccgagcgagactctgtctcaaaaaaaaaaaaaaaaggctgtggctgcatttggtccattggctgtaatatgctgattcctaattctctgggtaactttagtgtttgattagctactagaagttaggttaaacttttgtattttacaggctaactttaataatcttaaagtaaaacttaacatagttcatggaaaggaaatagaaattttaccctagtactcttttttttttttttttttttttttgaggcagagtctccctctgtcacccaggctggagtgcagtggtgggatcttggctgattgcaacctcctcctcctgggttcaagcaattcttgtgcctcagcctcccgagcagctgggactacaggcacgcaccaccacacctgactgatttttgtatttttagtagagacagggtttcgccatgttggccaggctggtcttgaactcctggcatcaagtgatcctcccatctgagcctcccagtgtgctgggattacagacgtgagtcactgtgcctggtctctagtattttttttttttttgagacggtctcactgttgccaggctggagtgcagtggcgcgatcctggctcactgcaacctccgcttcccggattcaagcgattttcctgcctcagcctcctgagtagctgggactatgggtgcacaccaccacgcccagctaatttttgtatttttagtagagacggggtttcaccatgttggccaatatggtctcaatctcttgacctcgtgatctgcccgtctcggcctcccaaagtgctgggattacaggcgtgagccactgtgcccagctgtactttttaagataagaattgcagggtatatatttttaccaacttaataacttataattttaaaaagctaattacttggctagaatataatgcgttacatattctttacactcagttcagtccatatctgaaaggcaaatagaattattttctgctagtacattgtgtagtccctatgttcctagtgtataaggactgttacctagttcacatttatctgggttgttgacagattttcctggtccctttggacagtgcatggccatgttggcaaaagctgtcaaaattgaaacattgacaccatgagaattgtgtgttttccagtctgctaaaatcaaaagtgggagggttcagtaaggtgaataacagaagcagagttttcggggtatctgttactcctcattcggcttttctgctctctgggggtctcaatttaaatataatgtgaaaattagttttacgaacctaaaaatgttgagtgattcatttcctggttttgttgttaatttctagatatttaaattaattgttagaagaaccccgttaaagaatgctttgcaaaacaacctccttatgtgctatgtctctgtttaatagtagttgagtttgtgtacatgagatcaatattttgaactatagctttttatgagttaaaaattgacggaacagttactgtgcacttgctgtgcaccatggtagtctcccaagtagtggtttttctgcatttcaatagtacatgagataggctgtgggtggcaaggtttcttgagaaagtgagggatgcacagttgggttttagaatacatcttgttcctccatgcccttccccaccaaaaggctggtagtcttgcatttgtatatagttagggtatttgatgtgttgcttccttgacagagttttgcaagaatttgcagatttaacaggaacaaaaacttacttaaaacaaaatctcttagtaaaagcatagtctagcaagatttagaatgatactttggctaacagtactttctctatatggagtgctttgtttccatagcctcacaagtatgttttcagataatagttgagttgaaaatgttgtcaatctcttgattttaaaaaatttacatatttaaagttgtatacttttgttcctacgtattttcagttgttcttaaagtttaataagtgacatttgaaaatgagtatatgtgtataaaaacaaaagtaggctaggcacggtggctcatgcctataatcctagcactttgggaggctgaggcaggcggatcacaaggtcaggagtttgagaccagcctgggcaatatggtgaaacccccctctactaaaaatacaaaaattagctgggtgtggtggtgcatgcctgtagtcccagctactcaggaggctgaggcaggagaatcgcttgaacccggaggtggcggttgcagtgagccgagattgcaccactgcagtccagcctgggcggcagagcgagactccatctcaaaaaaaaaaaacaaaaaaagaaaaagttaaaaaaaaacaaaaaacccccacaaaatgagtatatgtggcaacaagtcctattctcaaaaaaattattgtgtgctagttaagagcttaatgagtagccagtcggtattaaatatctgtttcagctatattttatctttaaaaattatctacagattttggaatgtgaaaaactagtgttttgtttcataggtatatactgtaggcattttaaaaataagagccagtgccagtggtttacagtgtacacaaggataatgttctcatgttctcttgatgtcagtatgactttaaagcatattatcaagaaataactaagtctgaaaaactgtggtaaataactggtactctaaaacctaagtttcttattactaaaaataagaaatggtaaaagtcaccctgtgctgttaattatatgagccactgaggtcctgacactgaattcttggtggtggataataatctcttctttttaattattggcttccaattctctctgcattgctggaaacaaaaatcatatatttcactattggtggtggggatgctgtcactgaaaaagtagacacattcatattgattttagaaataagttaaaatcaaaatttgcttctgctaaattagtagaggaccaatactgtttttctccttcatagtatgttttggtacttctacattgacattataacttttttttttttaaacagaaatagaagtttacattcttagaaaatttatgaaaatatgagcttttacctggtttgtgtgtgtgcgtatatatatacacatatttttaaatttcttacattgattttcaaattgaaagagaaccatttgtgaaagtatcttaacagagctcatgctttacattttacatgctacaaagttattttagtgccttaaattatttatgttgcttattaatgaaaattttggatacataattttttcaagacaaaggtaaaaataataaaccctttccttctgaggattaatgataaatataaactttaaaacgattaaaaaaatttttttagagacagggtcttgctctgttgcccagactgaagtgcagtggtgcagtcatagctcaatgaagcctcaaactcctgggcccaggcaaccctcctgcctcagccttttgagtagctgggacttcaggctcatgccaacatgcctaatttatcttatttttagtagagatgaggtctcaaactcctggcatctcttgccctctcaaagtgctggtactacaggcattagtcaccacacctgacacttaaaatcttttatatacaggtgtaagtgggtatctaacttaaagtgccaacgaatgtagttgaaagtttgtagttggcttagctaactagttaactaaattgattccattaaaaataagataagactgctcttagaatataatgatttttgttattcgttaaatataaatatatcactggatagtatatgttaatgacttgagatacgcattttaacatataatcacgttacttaaatgcctgcctttgaactgaaacttaacattatgaatttaaattaaagtttgactttagaggtaaatttctgtactttactaaagcagttcttaatataattctgagatttctaaaaattagtgtgccctaaagaattgaggtgtgtttttcttaactactgtaggcagtagatgtacagatgacttctgcatgcaaaaattaagccctagccattggtttacttcaactaatacttagttgccaattctctgtgtgtgattgaatttaaaactgcaaatggtactggtgatacattaactttttaggtgctaggtccactttgttacatttggttcagtagaaacattgatgttaccaatctcagaaagctaaaatatgtatgccaatccccaaattaggtaatttattcttaattttaagataaaagaatagaattcccttaaaattaaatgtggagtaaaatataccagctttaaaaaatattcacctttctgttagaagaatgaacataatattacatcttttaatttgcactatatatagattaatatttctgtgtatttctctgtgcccctactttgatggtatgcttttctgaacaaactagcagcacagttaactaagcactttgccccgtttgatgactgcctaattttctagattggaaaatattaaaaacttttatctccatatggccaatatatgattgtacctgttgtcatagctctcttatgtttaagcaagaaaaaccctattaagagtatttaaattagaatggaaggcacacagccagtatgattgaacactgttctaaaaattatttttaagacttgtagtaaggccaggtttggtggctcatggctgtaatcccagcccttaggaggccaaggtgggcggatcacttgtgctcaggagtttgagaccagcccgggcaacatggcaaaaccctgtctctacgaaaaatacaaaaatcagtcaggtgtggtggtgcttgcctgtagtcccagctatttgagaggctgaggcagggggatcacctagcctgggaggtcgaggctgcagtcatgatcgtgccattgcactccatcctgggcaacccagtgagaccctgtctctaaaacaaaaaaataaaaaaagaacttgtagtaaggatacaaaatgctcctattttgtgtgtgtcctttaattcatgatgtttttatattatggtaagcagctctcatttaagattttaataatgtaattaaacatgtacagaagacccagtctcagcttcacttgtataccctggaaatagactgaaaggtgttaaaatttaagataaaactcaaggttccagtttcttgactcacctttgagattcttttatgtttttgttgttttttaacaaaggtttcacgtccatattttaccatttttcttctcattctcccctggaggagggtgtgggaatcgatagtatataaatcacttttttcctaagtcaaagaagtaatttaaagctaacttcagtttaggctttaattccaggactagcaaactaaaatggttgcattaattgacaaacagatgctaatacctgtgtttaggcttgtcataatctctcctaattcctaatttaaaaattttaaaatttaattccattagaaaacaaaactgacttttaagaacaaaccaggattctagcccatattttaaaactgcatcctcagttttattcaaacagtctgatgtctgtttaaaaaaaaaaaaatctcaagctcataatctcaaacttcttgcacatggctttcccagtaaattactcttaccaatgcaacagactttaaagaagttgtgttttacaatgcagagagtggaggatgctttttatacattggtgagggagatccgacaatacagattgaaaaaaatcagcaaagaagaaaagactcctggctgtgtgaaaattaaaaaatgcattataatgtaatctggtaagtttaagttcagcacattaattttggcagaaagcagatgtcttttaaaggtaacaaggtggcaaccactttagaactacttaggtgtagtattctaacttgaagtattaaaagataagaaacttgtttccataattagtacatttatttttaatctagtgggaattaattataattgagacaattttgatggctgtagtagactaatctatatttggcataaagtctaatgatttaatgagtcttaagtaaactaaatatttggaaactgatatttacctttatttttaagggaaaagttttgagataatcagcagcttttttttttttttttttttttttagtagggagaaaaagatatgagctatagtagacagcagtaatattgaatggcccagaaggtgggaaaaagccactcttaaatgtattttttcttttggatattttacaagcaaataataacttctgcctaagttcgccatctcagtggcatcagcagcacagcactttcttatcccagtgagaaacctgggaattttaggatgactcctaccgccctcttttccccctggtttggaagtatccacaaattcctgtgacgttacattctgtgtcttttatgtcatcattagttcaggcccctatcatttcttgttggactgttagaacctcctatttggtttaccagttgctgccatcattcattgtgaaaccggagagatacactttaaagaaatgtcatttttggccgggcgcggtggctcacgcctgtaatcccagcactttgggaggcctaggcgggtgatcacctgaggtcaggagttcaagaccagcctggctaacatggtgaaaccctatttctactaaaaatacaaaaaattagccgggcgtggtggcacgtgcctgtaatcccagctacttgggaggctgaggcaggagaattgcttgaacctgggaggcagaggttgcagtgagctgagaatgcaccattgcactccagcttgagcaacaagagcgaaactctgtctcaaaaaaaaaaaaaaaaagtcattttagctatagaataaaatctcatgttccacatgtgttgcagatagtccttactaccttcccaccactccagctcttttttggtcttatatctaaaaacgtcatcttgcctgaatttcttttgttcttctataaataaataccatgttatttcctaccttcccttgagtcttggctcttgtttggaatgccagtatttttatccctagtcttactaattagctaacactctcatgattccccagtctcctactctctaaaaacctttctttaaacccttagactaggcatggagcccttcctgtgtattcccagaatactattcttaactattatatgcttcccatgttatgttgaaataactaacctcttctgtttcattcctatattacttgacagcaaaatcttagccagaattacatatttttaatctttgcacacccattgcctagtaaggttcctgggacatagtaactacccagtaaatatttattgcgtggaattctcattttcgtttctaaacccgtattaaactctgtcttgctcagaaaatacttcactaggtatcataaagttcatggcagagcttaagctttggatgcatattgtttgtaatatatcatgttcttaagaataggcaataaaattacagttttcaaaaactactacatttattatatttattacaagttggtgttctttattacatgaattttaggtatttcccaaaagtataaaatatacatttgaatagtagactcaatcccaaaagatactacgtggtgtactaatctactaaactcagaaacaaagcatgactggcattaatttttgttgaaatttatgaactctgaatgtttttgaatatcattctgtaaagcaatattttgcaattaaagcaattttgcatgttaaattttaccacaacctctaaaatattgcaaatttaacaatacagtttgaaaagttacacattttaaataacagtaccatgaccagatttaggtggtggttttaattttttattttctcctcctattgtctcaccattagatgattttaaaaatagaattgtttagagtaaaataagtgttatgctctaatttatatttaaaatgaaggtttaagcacgtactattctaaaatttctaatttgtgcaaattatgttttatacagtgactgtaggtgaatgtcacaattgtttgatgtgacgaatccttgtttttcagtacacgtggaagtaattcatataaaagagaagtatacttggtaattaaaaatttaaaattaaatacaatttaaaaaaaaatttatttgacaagctggctgtggtgtgtgtgcctgtagtatcagctgcttgggagcctgaggcaggaggattgcctgaccccaggagtttgaggttgaagggagctatgatggtgccatggcactgtagcctaggcaacagaaagagactccatctcttaaaaaaagtaaaaataaaaaaattttggcacagggacagtggctcacacttataatgccagaactttaggagtccacagcgcgaggactgcttgaggccaggagtttaagaccagactgggcaacgtaatgagaccccacctttaggaaataaatacataaataaaaatttgacaatgataaacatatataaattagcttttcttagtcctgaaaaagataatgttatgtgtatgtgtgagaatgattagttctcatatgagaaaaaaagaattcattgctctgtgtaggttgtgacatttccttcacgattgaaattaattaatttttttttattacttatttatttttaaaatagagacaggttcttgctgtgttgcccaggctggtctcaaactcctggcctcaagcagttctcctgcctcagcctcccaaattgctgtgactgtaggtgtgagccactgcactgggccaaaattacttaattttaacaagatgatgtagagaggagagttcattgcaacataagcctagaatctttgtcagaatcttaggaagtaatgttttcaaattctgtgttttcaccataaaatgtgtcttctctgtgtccatcacatggtttttcattgttttctgctttaccattttagtaccattggcatttttcttcattgtaaaagtagtagaaatggagtagattacataaggatgtgatcagagggaatttattcattcagggtaagggagttagatcctcttttaagattctatcacattctaagggtttatgattctaaactgtcaagtaaattgtcaagtgctggcaagctacagaataatttttattgtatcattggaaattttcccctctatatgtgttaaagagtttagcctgaagggatacatacacatacatatatgtaatcaaaccttgatggtattgtattgctgataaattatttcttaccacttttcctttctcctgtgggagaaacaaaagcatatgtttgtgtagtatcagtaatgatattagagagtgggaaacatcagtgagtgcagtttggggactttattggagactttcactagtgctcaaataaataatgctggtttttatcctactgtttgcttaatgtggactagcctcttattcccattctatgtttacctctcttaaaatattggtcacgctttcttgaattatagatctattaggaaaattcatgaactgtagctaattttcattgttcatgctccagatttattttgaaatatcgttaatcttagtagtacagtaaaggagaaataccacttaacattttttgtttttttttctttgagacagagtcatgctctgtcacccagtctggagtgcagtggtgctatctcggctcactgcaatgcacttcgcctctccgggttcagcaattctcctgcctcagcctcctgagtagctgggattacaggcacctgctaccacacccagctaatttttgtatttttagtagagacagggtttcaccatgttggccaggctggtctgaaactcctcacctcaagtgatccacccgtcttggcctcccaaagtgctgggattacaggcttgagccaccgcaccccgcccacttaacattttaaattaatttcaagataatatcacttgaatatttttacacatataatttttttaatacatttatttacacagtttataatatcctacaaagtgattacaatgagtaaaaacccagttttcattgttcctaaagtggcttgatttatacaacttaatgtgttgggtatttgtttctaagactccctctgctgtctaggtttggaagtattgtgaggttaacagattttctttttatagttactactcagttgaacaggctttaaaatacagagagaatcatattttttcttcattttttgcttttatttatatttttcttttaattggagacatgacaagaattgacttgtgtatggatcttgcataatttaagtactgcaggtttaaaatctactaccagtttgagagtgccatttttcacactgtagattattaggttgaaaagtattatggcttaaaatcgcttttagccattaaatttaaataaccttgctttaatcataaatagatggtggtcacaatgactaactgttaaactctttgaagacaggatatttggctttatatggcaagcttttgaatacaacagaaattaaaactttatgggatagaaagaatctcctccaaattggtaaactataagacctttcaaatgatttagctaatttctccacaaatctgaggtattagtgttttttttaaagtggtattctcctgtgttggggtcactttaaacctttttcttaatgataaatatatgaattgaaactaatcccttaatatatatcatttgaaaactgaaataatatgtttagatactgtttacttgttgataaattattggaataggatgttcgaatactgtttacttcttggtaaatttttaaatccaatggattttacgtaagtatagaactggagctcaaatactgttactgtgtgtgaagatatatgaacatagtttacagttgcatggcttatatctaaagtccagaaacataaggacaattaagtgtacacacacacacatgcatttggattttgatgacttaggtttgccaatgtggaaaaaatagtagcaaattaagttctcctgtgaaaaagtcgttaccttatttaaaattctgtgccattggttatccttgtcttttgtgaaaattagtgttcctgtttataatattgacaaaacacctatgcggatgacatttaagaattctaaaagtcctaatatatgtaatatatattcagttgcctgaagagaaacataaagaatcctttcttaatattttttccattaatgaaatttgttacctgtacacatgaagccatcgtatatattcacattttaatactttttatgtatttcagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccaggaactgtttgttttgtagtgaaccttgagtatgtgctgttaatataccaaattgggtgaaaaaataagggattcctttcaaaagttaagagaagtaagtgtgtaagaaattattttgcttattaaatgttcggtaaatggcattctcttgtcagtaaaatggagaaataagctaaaaataattggctaagtcctattaagttagaggattaagtgtattatattttcattcaaaattgggtgctcattaatttatgatcggtagtatagctaaattgctatgtttgtatcaaaattgagcataaagttgctgatactttctccgtatgaacagaagttgaaacctatttagttcagtagggcagctcagggattttttacacaacatgtatatcttcccattttaagttagaattattttacaacatctggtatacataaacagctggcactgatagctaaattaaagtagtaatgatcaattagttttgttggtatctgaataatagcgttgtttcatagctctgtatttcctaaggaagtacaaagcttctagctctttcattacaaattcgccctgtgcaataagttctttgatcttctctggattcttcacatctttgtttttaaggaaaatgttcttcaaacgctttttaaaatagtctgctccttttggatagtctcgtccaagatacagcagcttcaaaaagaaagattatatatttctaaacaatccatgtcatataataacatttttataaaattggcaacataattacttacatttttataaagttttagtacttctcctcttaaagaattggccattttcatttatcatgtaaattatccacttttatgcataacatacctaaagaaaggaaaatttttttgcaattagctgcattgtagtcttaaaaaaataaaaaaaggttatacacattgagaaaatggtaaccttttttacattcaataaatatttcttgataactttttcgttccacgtactgggatatagttataaacacttccgataaaattacctgctgtcataattgacgttttcctatgggagacataagcaaagacaattgtgattgtgagaagtcacatgaaggaaatgagaaagtggattgtcatcacagataggtacgtgtacctccttttatgccacagtggaatgagttaaactagatttaaattccagttgcataatgtacagattaattaaccttgctgagcctgagttttccttatcaacaaacaagagattatctttaccctgctctcaaggcaaggccagagccacttgaaggacattgagcagaagcctgatcaaatgctgatgggtgcttatccaaagggaggctgaaaactagcagaaactgggtgagttaagcaggttggaatagtagatgggcagtaagattggtggtgaagaggccaaatgaacaacctgtaagagggtgtccctgaggaacaggcaaaatcatgcttctttatgtgtaatgtgttaactctactttgtagaggaggctccaaact

SEQ ID NO: 18 - DNA, 38 bp, Artificial sequence
  Synthetic primer
cagagacgcagggatgagaagttctctctcagctagtg

SEQ ID NO: 19 - DNA, 38 bp, Artificial sequence
  Synthetic primer
cagagacgcagggatgagaagttctctctcgctgatgg

SEQ ID NO: 20 - DNA, 38 bp, Artificial sequence
  Synthetic primer
cagagacgcagggatgagaagttctctctcggtgacgt

SEQ ID NO: 21 - DNA, 46 bp, Artificial sequence; identical to SEQ ID NO: 4
  Synthetic primer

SEQ ID NO: 22 - DNA, 57 bp, Artificial sequence
  Synthetic primer
gctgaaaatgactgaatataaacttgtggtagttgacttctcatccctgcgtctctg

SEQ ID NO: 23 - DNA, 55 bp, Artificial sequence
  Synthetic primer
atgactgaatataaacttgtggtagttggagctacttctcatccctgcgtctctg

SEQ ID NO: 24 - DNA, 23 bp, Artificial sequence
  Synthetic primer
agttggagctggtggcgtaggca

SEQ ID NO: 25 - DNA, 25 bp, Artificial sequence
  Synthetic primer
tagttggagctggtggcgtaggcaa

SEQ ID NO: 26 - DNA, 25 bp, Artificial sequence
  Synthetic primer
ttggagctggtggcgtaggcaagag

(19) United States
(12) Patent Application Publication
(10) Pub. No.: US 2014/0186844 A1
(43) Pub. Date: Jul. 3, 2014

(54) POLYNUCLEOTIDE PRIMERS AND PROBES

(71) Applicants: Vladimir Makarov, Ann Arbor, MI (US); Laurie Kurihara, Ann Arbor, MI (US); Anjali Mishra, Ann Arbor, MI (US); Sergey V. Chupreta, Ann Arbor, MI (US)

(72) Inventors: Vladimir Makarov, Ann Arbor, MI (US); Laurie Kurihara, Ann Arbor, MI (US); Anjali Mishra, Ann Arbor, MI (US); Sergey V. Chupreta, Ann Arbor, MI (US)

(73) Assignee: SWIFT BIOSCIENCES, INC., Ann Arbor, MI (US)

(21) Appl. No.: 14/114,041

(22) PCT Filed: Apr. 26, 2012
(86) PCT No.: PCT/US2012/035206

§ 371 Date: Feb. 7, 2014

Related U.S. Application Data
(60) Provisional application No. 61/479,344, filed on Apr. 26, 2011.

(51) Int. Cl. C12Q 1/68

(52) U.S. Cl. CPC C12Q 1/6876
USPC 435/6.12; 536/24.33

(57) ABSTRACT

The present disclosure provides a novel technology that involves improved primer design. These primer pairs have a wide range of applications and provide high sensitivity and specificity.

### CROSS REFERENCE TO RELATED APPLICATIONS

This application claims the priority benefit under 35 U.S.C. §119(e) of U.S. Provisional Application No. 61/479,344, filed Apr. 26, 2011, which is incorporated by reference in its entirety.

### FIELD OF THE INVENTION

The disclosure relates to polynucleotide combinations and their use.

### SEQUENCE LISTING

This application contains, as a separate part of the disclosure, a Sequence Listing in computer-readable form (filename: 46157A_SeqListing.txt; created: Apr. 25, 2012; 74,958 bytes—ASCII text file) which is incorporated by reference in its entirety.

### BACKGROUND

Detection and amplification of nucleic acids play important roles in genetic analysis, molecular diagnostics, and drug discovery. Many such applications require specific, sensitive and inexpensive quantitative detection of certain DNA or RNA molecules, gene expression, DNA mutations or DNA methylation present in a small fraction of total polynucleotides. Many current methods use polymerase chain reaction, or PCR, and specifically, real-time PCR (quantitative, or qPCR) to detect and quantify very small amounts of DNA or RNA from clinical samples.

While the performance of current PCR assays is constantly improving, their sensitivity, specificity and cost are still far away from becoming a widely acceptable diagnostic test. Indeed, many PCR methods currently used in the art suffer from technical limitations that make the methods inadequate for many practical applications. For example, in instances where the target molecule has secondary structure that inhibits or even completely prevents binding of one or both primers to the target, amplification can be reduced or even non-existent, which, for example, from a diagnostic standpoint could give rise to a false negative despite use of a highly specific primer with binding properties that would be expected to be sensitive. Other challenges include low sensitivity of current real-time PCR assays in detection and discrimination of rare DNA molecules with a single base mutation in situations when they mixed with thousands of non-mutated DNA molecules, and ability to combine multiple mutation detection assays into one multiplex diagnostic assay.

There thus remains a need in the art for a development of amplification primers that combines high binding specificity with low synthesis cost that retain the ability to overcome technical problems recognized in the art, including novel application of PCR for diagnostics using next generation sequencing platforms.

### SUMMARY OF THE INVENTION

In one aspect, the disclosure provides a polynucleotide primer combination comprising a first polynucleotide (1) and a second polynucleotide (2), the first polynucleotide (1) comprising a first domain (a) having a sequence that is sufficiently complementary to a first target polynucleotide region (A), a second domain (c) comprising a unique polynucleotide sequence, and optionally a third domain (b) comprising a polymer sequence that is not sufficiently complementary to hybridize to a domain in the first polynucleotide (1), a domain in the second polynucleotide (2), or a region in the target polynucleotide, wherein domains in the first polynucleotide are arranged 5′-a-b-c-3′; the second polynucleotide (2) comprising a first domain (f) having a sequence that is sufficiently complementary to a sequence in a second target polynucleotide region (F), a second domain (d) comprising a polynucleotide sequence sufficiently complementary to (c) such that (c) and (d) will hybridize under appropriate conditions, and a third domain (e) comprising a polynucleotide sequence that is not sufficiently complementary to hybridize to a domain in the first polynucleotide (1), a domain in the second polynucleotide (2), or a region in the target polynucleotide, wherein domains in the second polymer are arranged 5′-d-e-f-3′, wherein under conditions in which region (A) specifically hybridizes to domain (a) and region (F) specifically hybridizes to domain (f), domain (c) hybridizes to domain (d) and neither domain (b) nor domain (e) hybridizes to a domain in the first polynucleotide (1), a domain in second polynucleotide (2) or a region in the target polynucleotide.

In another embodiment, the disclosure provides a polynucleotide primer combination comprising a first polynucleotide (1) and a second polynucleotide (2), the first polynucleotide (1) comprising a first domain (a) having a sequence that is sufficiently complementary to a first target polynucleotide region (A), a second domain (c) comprising a unique polynucleotide sequence, and a third domain (b) comprising a polymer sequence that is not sufficiently complementary to hybridize to a domain in the first polynucleotide (1), a domain in the second polynucleotide (2), or a domain in the target polynucleotide, wherein domains in the first polynucleotide are arranged 5′-a-b-c-3′; the second polynucleotide (2) comprising a first domain (f) having a sequence that is sufficiently complementary to a sequence in a second target polynucleotide region (F), a second domain (d) comprising a polynucleotide sequence sufficiently complementary to (c) such that (c) and (d) will hybridize under appropriate conditions, and optionally a third domain (e) comprising a polynucleotide sequence that is not sufficiently complementary to hybridize to a domain in the first polynucleotide (1), a domain in the second polynucleotide (2), or a domain in the target polynucleotide, wherein domains in the second polymer are arranged 5′-d-e-f-3′, wherein under conditions in which region (A) specifically hybridizes to domain (a) and region (F) specifically hybridizes to domain (f), domain (c) hybridizes to domain (d) and neither domain (b) nor domain (e) hybridizes to a domain in the first polynucleotide (1), a domain in second polynucleotide (2) or a domain in the target polynucleotide.

In some embodiments, polynucleotide primer combinations are provided wherein domain (c) is at least 70% complementary to domain (d), and in some aspects polynucleotide primer combinations are provided wherein domain (d) is at least 70% complementary to domain (c). In further aspects, domain (c) is at least 75%, at least 80%, at least 85%, at least 90%, at least 95% or more complementary to domain (d). Embodiments are also provided in which domain (d) is at least 75%, at least 80%, at least 85%, at least 90%, at least 95% or more complementary to domain (c). The disclosure also provides embodiments wherein domain (d) and domain (c) are sufficiently complementary to hybridize to each other in the absence of the template polynucleotide.

In various aspects, the polymer sequence of domain (b) is selected from the group consisting of a polynucleotide sequence, a polynucleotide sequence comprising at least one modified nucleotide, and a non-polynucleotide chemical polymer sequence. In one embodiment, the polymer sequence of domain (b) is a chemical polymer sequence that is hydrophilic.

In further embodiments, the polynucleotide sequence of domain (e) is selected from the group consisting of a polynucleotide sequence of naturally-occurring nucleotides and a polynucleotide sequence comprising at least one modified nucleotide, wherein the polynucleotide sequence of domain (e) is a template for polymerase extension.

Polynucleotide primer combinations of the disclosure include, in some aspects, those that further comprise a domain (g) positioned between domain (d) and domain (e) in the second polynucleotide, wherein domain (g) comprises a replication blocker. In still further aspects, the second polynucleotide further comprises a fourth domain (h) positioned between domain (f) and domain (e), wherein domain (h) comprises a replication blocker and wherein domain (h) is not part of domain (f). In one aspect, domain (h) is part of domain (e) while in various aspects, domain (h) is at a position in domain (e) that is 1, 2, 3, 4, or 5 nucleotides from the 3′ end of domain (e). Any moiety that can block extension of a polynucleotide by a polymerase enzyme is contemplated, and in certain aspects the replication blocker is selected from the group consisting of a modified base, an abasic site and a polymer. In some embodiments, the second polynucleotide (2) comprises both a domain (g) and a domain (h).

The disclosure also provides polynucleotide primer combinations in which the second polynucleotide (2) further comprises a domain (s) positioned 5′ of domain (d), wherein domain (s) comprises a sequence that is not sufficiently complementary to the first polynucleotide (1) or the second polynucleotide (2) to hybridize to the first polynucleotide (1) or the second polynucleotide (2) under conditions in which domain (a) specifically hybridizes to region (A), and optionally wherein domain (s) is sufficiently complementary to a polynucleotide extended from domain (f) to hybridize to the polynucleotide extended from domain (f) under conditions in which domain (a) specifically hybridizes to region (A). In some embodiments, domain (s) further comprises a detectable marker, and in various aspects domain (s) further comprises a quencher that quenches the detectable marker.

In some aspects, polynucleotide primer combinations are provided wherein domain (a) of the first polynucleotide (1) further comprises a domain (j), wherein domain (j) is contiguous with domain (a) and is positioned 3′ of domain (a), and wherein domain (j) is sufficiently complementary to a region (F*) in a non-target polynucleotide to hybridize under conditions in which domain (a) specifically hybridizes to region (A), wherein (i) region (F*) in the non-target polynucleotide differs from region (F) in the target polynucleotide at at least one nucleotide or (ii) region (F*) comprises a sequence that is identical to region (F).

In further aspects, domain (a) and domain (j) are not contiguous and are separated by a domain (p), wherein domain (p) is not sufficiently complementary to a domain in the first polynucleotide (1), a domain in the second polynucleotide (2), or the target polynucleotide to hybridize to the first polynucleotide (1), the second polynucleotide (2) or the target polynucleotide under conditions wherein domain (a) specifically hybridizes to region (A).

In any of the polynucleotide primer combinations, methods or uses described herein, the first polynucleotide (1) comprises a modified nucleotide. In some aspects, the modified nucleotide is in domain (a), and in further aspects the first polynucleotide (1) comprises a plurality of modified nucleotides in domain (a). In another aspect, the modified nucleotide is in domain (c) of the first polynucleotide (1).

The disclosure also provides polynucleotide primer combinations, methods or uses in which the second polynucleotide (2) comprises a modified nucleotide, and in some aspects the modified nucleotide is a nucleotide at a 5′ terminus or a 3′ terminus of second polynucleotide (2). Thus, in some embodiments, the modified nucleotide is the nucleotide at the 3′ terminus of second polynucleotide (2). In further aspects, the modified nucleotide is in domain (f) and in some embodiments the second polynucleotide (2) comprises a plurality of modified nucleotides in domain (f). With respect to the modified nucleotide, the disclosure provides embodiments wherein the modified nucleotide is the nucleotide that is 2, 3, 4, 5, 6, 7, 8, 9 or 10 nucleotides from the 3′ terminus of the second polynucleotide (2).

It is generally known in the art how to synthesize polynucleotides of various lengths. Accordingly, in various aspects the disclosure provides polynucleotide primer combinations wherein domain (d) is from about 5 bases in length to about 200 bases in length, about 5 bases in length to about 150 bases in length, about 5 bases in length to about 100 bases in length, about 5 bases in length to about 50 bases in length, about 5 bases in length to about 45 bases in length, about 5 bases in length to about 40 bases in length, about 5 bases in length to about 35 bases in length, about 5 bases in length to about 30 bases in length, about 5 bases in length to about 25 bases in length, about 5 bases in length to about 20 bases in length, about 5 bases in length to about 15 bases in length, about 10 to about 50 bases in length, about 10 bases in length to about 45 bases in length, about 10 bases in length to about 40 bases in length, about 10 bases in length to about 35 bases in length, about 10 bases in length to about 30 bases in length, about 10 bases in length to about 25 bases in length, about 10 bases in length to about 20 bases in length, or about 10 bases in length to about 15 bases in length.

In further aspects, polynucleotide primer combinations are provided wherein domain (a) is from about 10 bases in length to about 5000 bases in length, about 10 bases in length to about 4000 bases in length, about 10 bases in length to about 3000 bases in length, about 10 bases in length to about 2000 bases in length, about 10 bases in length to about 1000 bases in length, about 10 bases in length to about 500 bases in length, about 10 bases in length to about 250 bases in length, about 10 bases in length to about 200 bases in length, about 10 bases in length to about 150 bases in length, about 10 bases in length to about 100 bases in length, about 10 bases in length to about 95 bases in length, about 10 bases in length to about 90 bases in length, about 10 bases in length to about 85 bases in length, about 10 bases in length to about 80 bases in length, about 10 bases in length to about 75 bases in length, about 10 bases in length to about 70 bases in length, about 10 bases in length to about 65 bases in length, about 10 bases in length to about 60 bases in length, about 10 bases in length to about 55 bases in length, about 10 bases in length to about 50 bases in length, about 10 bases in length to about 45 bases in length, about 10 bases in length to about 40 bases in length, about 10 bases in length to about 35 bases in length, about 10 bases in length to about 30 bases in length, or about 10 bases in length to about 100 bases in length.

In still further aspects, polynucleotide primer combinations are provided wherein domain (c) is from about 5 bases in length to about 200 bases in length, about 5 bases in length to about 150 bases in length, about 5 bases in length to about 100 bases in length, about 5 bases in length to about 50 bases in length, about 5 bases in length to about 45 bases in length, about 5 bases in length to about 40 bases in length, about 5 bases in length to about 35 bases in length, about 5 bases in length to about 30 bases in length, about 5 bases in length to about 25 bases in length, about 5 bases in length to about 20 bases in length, about 5 bases in length to about 15 bases in length, about 10 to about 50 bases in length, about 10 bases in length to about 45 bases in length, about 10 bases in length to about 40 bases in length, about 10 bases in length to about 35 bases in length, about 10 bases in length to about 30 bases in length, about 10 bases in length to about 25 bases in length, about 10 bases in length to about 20 bases in length, or about 10 bases in length to about 15 bases in length.

Polynucleotide primer combinations of the disclosure, in various embodiments, are provided in which the first polynucleotide (1) and/or the second polynucleotide (2) is DNA, modified DNA, RNA, modified RNA, peptide nucleic acid (PNA), or combinations thereof.

In some aspects, polynucleotide primer combinations of the disclosure further comprise an extension blocking group attached to the first polynucleotide (1) at its 3′ end which blocks extension from a DNA polymerase. In various embodiments, the extension blocking group is selected from the group consisting of a 3′ phosphate group, a 3′ amino group, a dideoxy nucleotide, and an inverted deoxythymidine (dT).

Polynucleotide primer combinations are also provided wherein the first target polynucleotide region (A) and the second target polynucleotide region (F) are separated by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 10000, 20000, 30000, 40000, 50000, 60000, 70000, 80000, 90000 or 100000 or more bases in the target polynucleotide. In further aspects, polynucleotide region (A) and polynucleotide region (F) and/or (F*) are separated by at least 110 kilobases, at least 120 kilobases, at least 130 kilobases, at least 140 kilobases, at least 150 kilobases, at least 160 kilobases, at least 170 kilobases, at least 180 kilobases, at least 190 kilobases, at least 200 kilobases, at least 210 kilobases, at least 220 kilobases, at least 230 kilobases, at least 240 kilobases, at least 250 kilobases, at least 260 kilobases, at least 270 kilobases, at least 280 kilobases, at least 290 kilobases, at least 300 kilobases, at least 310 kilobases, at least 320 kilobases, at least 330 kilobases, at least 340 kilobases, at least 350 kilobases, at least 360 kilobases, at least 370 kilobases, at least 380 kilobases, at least 390 kilobases, at least 400 kilobases, at least 410 kilobases, at least 420 kilobases, at least 430 kilobases, at least 440 kilobases, at least 450 kilobases, at least 460 kilobases, at least 470 kilobases, at least 480 kilobases, at least 490 kilobases, at least 500 kilobases, at least 510 kilobases, at least 520 kilobases, at least 530 kilobases, at least 540 kilobases, at least 550 kilobases, at least 560 kilobases, at least 570 kilobases, at least 580 kilobases, at least 590 kilobases, at least 600 kilobases, at least 610 kilobases, at least 620 kilobases, at least 630 kilobases, at least 640 kilobases, at least 650 kilobases, at least 660 kilobases, at least 670 kilobases, at least 680 kilobases, at least 690 kilobases, at least 700 kilobases, at least 710 kilobases, at least 720 kilobases, at least 730 kilobases, at least 740 kilobases, at least 750 kilobases, at least 760 kilobases, at least 770 kilobases, at least 780 kilobases, at least 790 kilobases, at least 800 kilobases, at least 810 kilobases, at least 820 kilobases, at least 830 kilobases, at least 840 kilobases, at least 850 kilobases, at least 860 kilobases, at least 870 kilobases, at least 880 kilobases, at least 890 kilobases, at least 900 kilobases, at least 910 kilobases, at least 920 kilobases, at least 930 kilobases, at least 940 kilobases, at least 950 kilobases, at least 960 kilobases, at least 970 kilobases, at least 980 kilobases, at least 990 kilobases, at least 1 megabase, at least 1.5 megabases, at least 2 megabases, at least 2.5 megabases, at least 3 megabases, at least 3.5 megabases, at least 4 megabases, at least 4.5 megabases, at least 5 megabases, at least 5.5 megabases, at least 6 megabases, at least 6.5 megabases, at least 7 megabases, at least 7.5 megabases, at least 8 megabases, at least 8.5 megabases, at least 9 megabases, at least 9.5 megabases, at least 10 megabases, at least 10.5 megabases, at least 11 megabases, at least 11.5 megabases, at least 12 megabases, at least 12.5 megabases, at least 13 megabases, at least 13.5 megabases, at least 14 megabases, at least 14.5 megabases, at least 15 megabases, at least 15.5 megabases, at least 16 megabases, at least 16.5 megabases, at least 17 megabases, at least 17.5 megabases, at least 18 megabases, at least 18.5 megabases, at least 19 megabases, at least 19.5 megabases, at least 20 megabases or more in the target and/or non-target polynucleotide.

Also provided are polynucleotide primer combinations wherein domain (f) is about 2, 3, 4, 5, 6, 7, 8, 9, 10, 11, 12, 13, 14, 15, 16, 17, 18, 19 or 20 bases in length, and in one aspect domain (f) is 8 bases in length. In some aspects, the sequence of domain (f) is 100% complementary to the sequence of the second target polynucleotide region (F), while in further aspects the sequence of domain (f) comprises a mismatch with the sequence of the non-target polynucleotide region (F*). In related aspects, polynucleotide primer combinations are provided wherein the mismatch in the sequence of domain (f) with respect to the sequence of the non-target polynucleotide region (F*) is not a 3′ base mismatch, while in further aspects polynucleotide primer combinations are provided wherein the mismatch in the sequence of domain (f) with respect to the sequence of the non-target polynucleotide region (F*) is a 3′ base mismatch. In an aspect, the mismatch is a mutation in the target polynucleotide, and in further embodiments the mutation in the target polynucleotide is selected from the group consisting of an insertion, a deletion, a substitution and an inversion. In some embodiments, region (F) and region (F*) comprise an identical sequence. Thus, in some aspects, the sequence of domain (f) is 100% complementary to the sequence of region (F*).

The disclosure further provides a polynucleotide primer combination further comprising a blocker polynucleotide that comprises a 5′ terminus and a 3′ terminus.

In aspects involving a blocker polynucleotide, it is contemplated that the blocker polynucleotide comprises a nucleotide sequence that is sufficiently complementary to region (F) such that the blocker polynucleotide will hybridize to all or part of region (F) under appropriate conditions. In some embodiments, a polynucleotide primer combination is provided wherein a nucleotide at the 3′ end of the second polynucleotide (2) and a nucleotide at the 5′ terminus of the blocker polynucleotide overlap. In further embodiments, a polynucleotide primer combination is provided wherein the blocker polynucleotide has a sequence that overlaps (f) over the entire length of (f). In every aspect of the disclosure, the blocker polynucleotide is perfectly complementary to a region of a non-target polynucleotide and not perfectly complementary to a region of a target polynucleotide. Also in every aspect of the disclosure, domain (f) is perfectly complementary to a region in a target polynucleotide.

Polynucleotide primer combinations are also provided wherein the nucleotide at the 3′ end of the second polynucleotide (2) and the nucleotide at the 5′ terminus of the blocker polynucleotide are different. In one aspect, an internal nucleotide of the second polynucleotide (2) and an internal nucleotide of the blocker polynucleotide overlap. Thus, in various embodiments, the nucleotide of the second polynucleotide (2) that overlaps with the nucleotide of the blocker polynucleotide is 1, 2, 3, 4, 5, 6, 7, 8, 9, 10, 11, 12, 13, 14, 15, 16, 17, 18 or 19 nucleotides upstream of the 3′ end of the second polynucleotide (2). In further aspects, more than one nucleotide of domain (f) is different from more than one nucleotide of the blocker polynucleotide.

In any of the polynucleotide primer combinations of the disclosure, it is contemplated that the second polynucleotide (2), the first polynucleotide (1), and/or the blocker polynucleotide comprises a modified nucleotide.

The disclosure further provides polynucleotide primer combinations wherein the blocker polynucleotide is sufficiently complementary to hybridize under appropriate conditions to a third target polynucleotide region (X), wherein region (X) is between region (A) and region (F) in the target polynucleotide. In some aspects, region (X) comprises a sequence that is at least about 1 nucleotide to about 100 kilobases in length. In further embodiments, region (X) comprises a sequence that is at least about 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, at least 590, at least 600, at least 610, at least 620, at least 630, at least 640, at least 650, at least 660, at least 670, at least 680, at least 690, at least 700, at least 710, at least 720, at least 730, at least 740, at least 750, at least 760, at least 770, at least 780, at least 790, at least 800, at least 810, at least 820, at least 830, at least 840, at least 850, at least 860, at least 870, at least 880, at least 890, at least 900, at least 910, at least 920, at least 930, at least 940, at least 950, at least 960, at least 970, at least 980, at least 990 nucleotides, or at least 1, at least 10, at least 20, at least 30, at least 40, at least 50, at least 60, at least 70, at least 80, at least 90 or at least 100 or more kilobases in length.

A polynucleotide primer combination of the disclosure, in various aspects, further comprises a first rigid frame blocker polynucleotide (RF1) and a second rigid frame blocker polynucleotide (RF2). In these aspects, polynucleotide (RF1) comprises a domain (q) that is sufficiently complementary to a region (Xq) in a non-target polynucleotide to allow hybridization between domain (q) and region (Xq) under appropriate conditions, a domain (t) that is sufficiently complementary to a region (Xt) in the non-target polynucleotide to allow hybridization between domain (t) and region (Xt), and a domain (r) that is sufficiently complementary to a domain (v) in polynucleotide (RF2) to allow hybridization between domain (r) and domain (v) under appropriate conditions; and wherein polynucleotide (RF2) comprises a domain (u) that is sufficiently complementary to a region (Xu) in a non-target polynucleotide to allow hybridization between domain (u) and region (Xu) under appropriate conditions, a domain (w) that is sufficiently complementary to a region (Xw) in the non-target polynucleotide to allow hybridization between domain (w) and region (Xw), and domain (v) that is sufficiently complementary to domain (r) in polynucleotide (RF1) to allow hybridization between domain (r) and domain (v) under appropriate conditions, and wherein when domain (q) is specifically hybridized to region (Xq), and domain (t) is specifically hybridized to region (Xt), and domain (u) is specifically hybridized to region (Xu), and domain (w) is specifically hybridized to region (Xw), and domain (r) is specifically hybridized to domain (v), and domain (a) is specifically hybridized to region (A), domain (f) will not hybridize to region (F), and wherein region (Xq), region (Xt), region (Xu), and region (Xw) are not in the target polynucleotide.

In various aspects of the disclosure, a polynucleotide primer combination is provided wherein the blocker polynucleotide is sufficiently complementary to hybridize under appropriate conditions to a fourth target polynucleotide region (Y), wherein region (Y) is 5′ of region (F) in the target polynucleotide. In further embodiments, the blocker polynucleotide is from at least about 1 to about 100 nucleotides or more in length. In various aspects, the blocker polynucleotide is at least about 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100 or more nucleotides in length. In one aspect, the blocker polynucleotide is about 20 bases in length.

In some aspects of the disclosure, polynucleotide primer combinations are provided that further comprise a probe polynucleotide, the probe polynucleotide comprising a nucleotide sequence that is complementary to the fourth target polynucleotide region (Y). In some embodiments, the probe polynucleotide comprises a label and a quencher. In any of the embodiments comprising a probe polynucleotide, it is contemplated that the first polynucleotide, the second polynucleotide and/or the probe polynucleotide comprises a modified nucleotide.

In certain polynucleotide primer combinations of the disclosure, region (A) is 3′ to region (F) in the target polynucleotide, while in other polynucleotide primer combinations, region (A) is 5′ to region (F) in the target polynucleotide.

In further embodiments, a polynucleotide primer combination is provided wherein at least one nucleotide in domain (a) overlaps at least one nucleotide in domain (f). In some aspects, a polynucleotide primer combination of the disclosure is provided wherein domain (a) comprises a label. In an aspect, the label is quenchable. Accordingly, in certain aspects a polynucleotide primer combination is provided wherein domain (a) comprises a quencher. In specific embodiments, the quencher is selected from the group consisting of Black Hole Quencher 1, Black Hole Quencher-2, Iowa Black FQ, Iowa Black RQ, a G-Base, and Dabcyl.

A polynucleotide primer combination is also provided that further comprises a reverse primer, wherein the reverse primer comprises a polynucleotide sequence complementary to a sequence extended from domain (f).

The disclosure further provides a method of detecting a target polynucleotide in a sample with a primer combination, the primer combination comprising a first polynucleotide (1) and a second polynucleotide (2), the first polynucleotide (1) comprising a first domain (a) having a sequence that is sufficiently complementary to a first target polynucleotide region (A), a second domain (c) comprising a unique polynucleotide sequence, and a third domain (b) comprising a sequence that is not sufficiently complementary to hybridize to a domain in the first polynucleotide (1), a domain in the second polynucleotide (2), or a domain in the target polynucleotide, or the third domain comprises a chemical polymer, the second polynucleotide (2) comprising a first domain (f) that is fully complementary to a second target polynucleotide region (F), a second domain (d) comprising a polynucleotide sequence sufficiently complementary to domain (c) such that domain (c) and domain (d) will hybridize under appropriate conditions, and a third domain (e) comprising a sequence that is not sufficiently complementary to hybridize to a domain in the first polynucleotide (1), a domain in the second polynucleotide (2), or a domain in the target polynucleotide, domain (f) having a sequence that is not fully complementary to a non-target polynucleotide in the sample and the method comprising the steps of: contacting the sample with the primer combination and a polymerase under conditions that allow extension of a sequence from domain (f) which is complementary to the target polynucleotide when the second target polynucleotide region (F) is present in the sample and detecting the sequence extended from domain (f) indicating the second target polynucleotide region (F) is present in the sample.

In some aspects, the method provides a change in sequence detection from a sample with a non-target polynucleotide region compared to sequence detection from a sample with a second target polynucleotide region (F).

Also provided is a method of detecting a target polynucleotide in a sample with any of the polynucleotide primer combinations of the disclosure wherein the second polynucleotide (2) comprises a first domain that is fully complementary to region (F) and wherein domain (f) is not fully complementary to a non-target polynucleotide region in the sample, the method comprising the steps of: contacting the sample with the primer combination and a polymerase under conditions that allow extension of a sequence from domain (f) which is complementary to the target polynucleotide when the second target polynucleotide region (F) is present in the sample and detecting the sequence extended from domain (f). In various embodiments, the method provides a change in sequence detection from a sample with a non-target polynucleotide region compared to sequence detection from a sample with a second target polynucleotide region (F).

In each of these methods, an embodiment is provided wherein the detecting step is carried out using polymerase chain reaction. In these embodiments, aspects are provided wherein the polymerase chain reaction utilizes the second polynucleotide (2) of the primer combination and a reverse primer, the reverse primer having a sequence complementary to the sequence extended from domain (f). In further aspects, the polymerase chain reaction utilizes a reverse primer complementary to the sequence extended from domain (f) and a forward primer having a sequence complementary to the strand of the target polynucleotide to which domain (f) hybridizes.

In another aspect of these methods, detection is carried out in real time.

The disclosure further provides a method of initiating polymerase extension using a primer combination and a target polynucleotide as template in a sample, the primer combination comprising a first polynucleotide (1) and a second polynucleotide (2), the first polynucleotide (1) comprising a first domain (a) having a sequence that is sufficiently complementary to a first target polynucleotide region (A), a second domain (c) comprising a unique polynucleotide sequence, and a third domain (b) comprising a sequence that is not sufficiently complementary to hybridize to a domain in the first polynucleotide (1), a domain in the second polynucleotide (2), or a domain in the target polynucleotide, or the third domain comprises a chemical polymer, the second polynucleotide (2) comprising a first domain (f) that is fully complementary to a second target polynucleotide region (F), a second domain (d) comprising a polynucleotide sequence sufficiently complementary to domain (c) such that domain (c) and domain (d) will hybridize under appropriate conditions, and a third domain (e) comprising a sequence that is not sufficiently complementary to hybridize to a domain in the first polynucleotide (1), a domain in the second polynucleotide (2), or a domain in the target polynucleotide, domain (f) having a sequence that is not fully complementary to a non-target polynucleotide in the sample, and wherein the sample comprises a mixture of (i) a target polynucleotide that has a sequence (F) that is fully complementary to the sequence in domain (f) and (ii) a non-target polynucleotide that has a sequence (F*) that is not fully complementary to (f), wherein the sequence of (F) is identical to the sequence of (F*) except for at least a one nucleotide difference, the method comprising the step of contacting the sample with the primer combination and a polymerase under conditions that allow extension of a sequence from domain (f) and complementary to the target polynucleotide strand when domain (f) contacts region (F). In some aspects of methods of the disclosure, the sequence in the region (F) in the target polynucleotide differs from the sequence in the region (F*) in the non-target polynucleotide at least at one base position. In a further aspect, a method is provided further comprising the step of detecting the sequence extended from domain (f), wherein detection indicates the presence of the target polynucleotide in the sample.

In one aspect of any of the methods described herein, extension of a sequence from domain (f) causes displacement of domain (a) from the target polynucleotide. In another aspect, extension of a sequence from domain (f) causes degradation of domain (a).

Also provided is a method of initiating polymerase extension using any of the polynucleotide primer combinations described herein and a target polynucleotide as template in a sample, wherein the second polynucleotide (2) comprises a first domain (f) that is fully complementary to a first target polynucleotide region (F) and wherein domain (f) is not fully complementary to a non-target polynucleotide in the sample, the method comprising the steps of: contacting the sample with the primer combination and a polymerase under conditions that allow extension of a sequence from domain (f) which is complementary to the target polynucleotide when the target polynucleotide is present in the sample. In some aspects, methods described herein further comprise the step of detecting the sequence extended from domain (f), indicating the presence of the target polynucleotide in the sample.

The disclosure further provides a method of amplifying a target polynucleotide in a sample using a polynucleotide primer combination, the primer combination comprising a first polynucleotide (1) and a second polynucleotide (2), the first polynucleotide (1) comprising a first domain (a) having a sequence that is sufficiently complementary to a first target polynucleotide region (A), a second domain (c) comprising a unique polynucleotide sequence, and a third domain (b) comprising a sequence that is not sufficiently complementary to hybridize to a domain in the first polynucleotide (1), a domain in the second polynucleotide (2), or a domain in the target polynucleotide, or the third domain comprises a chemical polymer, the second polynucleotide (2) comprising a first domain (f) that is fully complementary to a second target polynucleotide region (F), a second domain (d) comprising a polynucleotide sequence sufficiently complementary to domain (c) such that domain (c) and domain (d) will hybridize under appropriate conditions, and a third domain (e) comprising a sequence that is not sufficiently complementary to hybridize to a domain in the first polynucleotide (1), a domain in the second polynucleotide (2), or a domain in the target polynucleotide, domain (f) having a sequence that is not fully complementary to a non-target polynucleotide in the sample and wherein the sample comprises a mixture of (i) a target polynucleotide that has a sequence in region (F) that is fully complementary to the sequence in domain (f) and (ii) one or more non-target polynucleotides that are not fully complementary to domain (f); the method comprising the steps of: (a) contacting the sample with the primer combination and a polymerase under conditions that allow extension of a sequence from domain (f) which is complementary to the target polynucleotide when the target polynucleotide is present in the sample, (b) denaturing the sequence extended from domain (f) from the target polynucleotide, and (c) repeating step (a) in the presence of a reverse primer having a sequence complementary to a region in the sequence extended from domain (f) in step (b) to amplify the target polynucleotide, wherein extension and amplification of the target polynucleotide occurs when region (F) is fully complementary to the sequence in the domain (f) but is less efficient or does not occur when region (F) in the target polynucleotide is not fully complementary to the sequence in domain (f).

Also provided is a method of amplifying a target polynucleotide in a sample using any of the polynucleotide primer combinations of the disclosure, wherein the second polynucleotide (2) comprises a first domain (f) that is fully complementary to a first target polynucleotide region (F) and wherein domain (f) is not fully complementary to a non-target polynucleotide in the sample, the method comprising the steps of: (a) contacting the sample with the primer combination and a polymerase under conditions that allow extension of a sequence from domain (f) which is complementary to the target polynucleotide when the target polynucleotide is present in the sample, (b) denaturing the sequence extended from domain (f) from the target polynucleotide, and (c) repeating step (a) in the presence of a reverse primer having a sequence complementary to a region in the sequence extended from (f) in step (b) to amplify the target polynucleotide, wherein extension and amplification of the target polynucleotide occurs when region (F) is fully complementary to the sequence in domain (f) but is less efficient or does not occur when the first region in the target polynucleotide is not fully complementary to the sequence in domain (f). In some aspects, the reverse primer has a sequence that is fully complementary to a region in the sequence extended from domain (f), while in further aspects the reverse primer is a primer combination comprising a first polynucleotide (3) and a second polynucleotide (4), the first polynucleotide (3) comprising a first domain (a2) having a sequence that is sufficiently complementary to a first region (A2) in the sequence extended from domain (f) in step (a), a second domain (c2) comprising a unique polynucleotide sequence, and a third domain (b2) comprising a sequence that is not sufficiently complementary to hybridize to a domain in (3), a domain in (4), a domain in the first polynucleotide (1), a domain in the second polynucleotide (2), a domain in the target polynucleotide or a domain in the sequence extended from domain (f), or the third domain comprises a chemical polymer, the second polynucleotide (4) comprising a first domain (f2) that is fully complementary to a second region (F2) in the sequence extended from domain (f) in step (a), a second domain (d2) comprising a polynucleotide sequence sufficiently complementary to domain (c2) such that domain (c2) and domain (d2) will hybridize under appropriate conditions, and a third domain (e2) comprising a sequence that is not sufficiently complementary to hybridize to a domain in (3), a domain in (4), a domain in the first polynucleotide (1), a domain in the second polynucleotide (2), a domain in the target polynucleotide or a domain in the sequence extended from domain (f).

It will be understood that any of the polynucleotide primer combinations disclosed herein may be used in any of the methods likewise disclosed herein. Accordingly, in each method of the disclosure, an aspect is provided wherein the reverse primer is a primer combination as disclosed herein.

In each of the methods of the disclosure, the method further comprises the step of detecting a product amplified in the method. In some aspects, detection is carried out using polymerase chain reaction, and in further aspects detection is carried out in real time. In some embodiments, detection is carried out through the use of a detectable marker.

### BRIEF DESCRIPTION OF THE DRAWINGS

FIG. 1 is a schematic representing a primer combination of the disclosure.

FIG. 2 is a schematic representing a primer combination in which the relative positioning of domain (a) and domain (f) is inverted.

FIG. 3 is a schematic of mutant allele selection and extension by a polynucleotide primer combination of the disclosure for use in detecting a mutation in a target polynucleotide versus a non-target polynucleotide.

FIG. 4 depicts target polynucleotide discrimination with a polynucleotide primer combination of the disclosure and a blocker polynucleotide.

FIG. 5 depicts three forward polynucleotide primer combinations of the disclosure for multiplexed PCR that have flexible domains (e2), (e4) and (e6), each with the same universal sequence ‘e’.

FIG. 6 is a depiction of PCR with a polynucleotide primer combination of the disclosure and a conventional (standard) reverse primer.

FIG. 7 depicts the steps of PCR with a polynucleotide primer combination of the disclosure and a conventional reverse primer.

FIG. 8 is a depiction of PCR with two polynucleotide primer combinations of the disclosure.

FIG. 9 depicts a detailed protocol of PCR with two polynucleotide primer combinations of the disclosure.

FIG. 10 depicts a polynucleotide primer combination of the disclosure that further comprises a replication blocking domain (g).

FIG. 11 depicts PCR with two polynucleotide primer combinations of the disclosure, each comprising a replication-blocking domain (g), and two conventional primers.

FIG. 12 depicts a detailed protocol of PCR with two polynucleotide primer combinations of the disclosure, each comprising a replication-blocking domain (g), and two conventional primers.

FIG. 13 depicts a polynucleotide primer combination of the disclosure in which region (A) is 5′ to region (F) in the target polynucleotide, for use in detecting a mutation in a target polynucleotide versus a non-target polynucleotide.

FIG. 14 depicts a polynucleotide primer combination of the disclosure in which region (A) is 5′ to region (F) in the target polynucleotide, for use in detecting a mutation in a target polynucleotide versus a non-target polynucleotide wherein at least a portion of domain (a) acts as a blocker polynucleotide.

FIG. 15 depicts a polynucleotide primer combination wherein domain (a) further comprises a detectable marker and a quencher that quenches the detectable marker, for use in detecting a mutation in a target polynucleotide versus a non-target polynucleotide.

FIG. 16 depicts a polynucleotide primer combination wherein domain (a) further comprises a detectable marker and a quencher that quenches the detectable marker, and wherein domain (a) further comprises a blocker polynucleotide domain (a_{5′}), for use in detecting a mutation in a target polynucleotide versus a non-target polynucleotide.

FIG. 17 depicts a polynucleotide primer combination that further comprises a replication blocker domain within the second polynucleotide (2).

FIG. 18 depicts a PCR method using a polynucleotide primer combination of the disclosure in which region (A) is 5′ to region (F) in the target polynucleotide, without a replication blocker domain.

FIG. 19 depicts a polynucleotide primer combination of the disclosure in which region (A) is 5′ to region (F) in the target polynucleotide and is used for PCR wherein the second polynucleotide (2) and the fourth polynucleotide (4) comprise a replication blocker domain (g).

FIG. 20 depicts the steps of PCR when using polynucleotide primer combinations of the disclosure in which region (A) is 5′ to region (F) in the target polynucleotide, and wherein the second and fourth polynucleotides (2) and (4) each comprise a replication blocker domain (g).

FIG. 21 depicts potential interactions between polynucleotide primer combinations of the disclosure in which region (A) is 5′ to region (F) in the target polynucleotide, wherein the second polynucleotide (2) and the fourth polynucleotide (4) further comprise a replication blocker domain (h) at different temperatures, and illustrates the lack of amplifiable primer-dimer formation by the depicted polynucleotide primer combinations.

FIG. 22 depicts polynucleotide primer combinations of the disclosure in which region (A) is 5′ to region (F) in the target polynucleotide, wherein the second polynucleotide (2) and fourth polynucleotide (4) further comprise a replication blocker domain (h) and their use in PCR.

FIG. 23 A-G depicts the steps of PCR when using polynucleotide primer combinations of the disclosure in which region (A) is 5′ to region (F) in the target polynucleotide, wherein the second and fourth polynucleotides (2) and (4) each comprise a replication blocker domain (h).

FIG. 24 depicts a polynucleotide primer combination in which the first polynucleotide (1) comprises a self-blocking domain (j).

FIG. 25 depicts a polynucleotide primer combination in which the first polynucleotide (1) comprises a self-blocking domain (j) and a domain (p).

FIG. 26 shows a polynucleotide primer combination for detecting a small deletion.

FIG. 27 shows a polynucleotide primer combination for detecting a small deletion, and further comprising a blocker polynucleotide.

FIG. 28 depicts a polynucleotide primer combination for detecting a large deletion and further comprising a blocker polynucleotide that prevents looping-out and priming of non-target polynucleotide.

FIG. 29 depicts a rigid frame blocker polynucleotide and its interaction with a non-target polynucleotide.

FIG. 30 shows the inhibition of non-target polynucleotide extension by a polynucleotide primer combination of the disclosure using a rigid frame blocker polynucleotide, wherein the deletion is at least 50 bases in length.

FIG. 31 depicts deletion detection using a junction-specific polynucleotide primer combination.

FIG. 32 depicts small insertion detection using an insertion-specific polynucleotide primer combination.

FIG. 33 depicts small insertion detection using an insertion-specific polynucleotide primer combination and a blocker polynucleotide.

FIG. 34 shows large insertion detection using an insertion-specific polynucleotide primer combination.

FIG. 35 depicts results of a qPCR analysis of KRAS mutant G12A using synthetic KRAS template when region (F) is 5′ to region (A) in the target polynucleotide.

FIG. 36 depicts results of a qPCR analysis of KRAS mutant G12R using synthetic KRAS template when region (F) is 5′ to region (A) in the target polynucleotide.

FIG. 37 depicts results of a qPCR analysis of KRAS mutant G12C using synthetic KRAS template when region (F) is 5′ to region (A) in the target polynucleotide.

FIG. 38 depicts results of a qPCR analysis of KRAS mutant G12D using synthetic KRAS template when region (F) is 5′ to region (A) in the target polynucleotide.

FIG. 39 depicts results of a qPCR analysis of KRAS mutant G12V using synthetic KRAS template when region (F) is 5′ to region (A) in the target polynucleotide.

FIG. 40 depicts results of a qPCR analysis of KRAS mutant G12S using synthetic KRAS template when region (F) is 5′ to region (A) in the target polynucleotide.

FIG. 41 depicts results of a qPCR analysis of KRAS mutant G13D using synthetic KRAS template when region (F) is 5′ to region (A) in the target polynucleotide.

FIG. 42 shows qPCR curves for G12V KRAS mutant sample containing 50 ng SW480 DNA (14,000 mutant DNA copies) versus 50 ng (14,000 copies) of wild-type (WT) genomic DNA when region (A) is 5′ to region (F) in the target polynucleotide (opposite to orientation of primer domains in Example 1).

FIG. 43 shows qPCR curves for a wild type genomic DNA sample versus 10 and 100 copies of KRAS G12D in a background of wild-type genomic DNA.

FIG. 44 depicts qPCR curves for wild type genomic DNA versus 10 and 100 copies of KRAS G12S in a background of wild-type genomic DNA.

FIG. 45 shows qPCR curves for wild type genomic DNA versus 10 and 100 copies of KRAS G13D in a background of wild-type genomic DNA.

### DETAILED DESCRIPTION OF THE INVENTION

The disclosure is based on the discovery of a discontinuous polynucleotide design that overcomes problems encountered during the hybridization of polynucleotides, and in particular, amplification primer hybridization to a target polynucleotide. These problems include but are not limited to a low specificity to single-base changes in a target polynucleotide.

Polynucleotide combinations described herein offer an advantage over both standard PCR primers and long PCR primers when using polynucleotide templates that are difficult to amplify efficiently. Such templates include, for example, those that contain a degree of secondary structure formed through internal self-hybridization giving rise to, for example, loops, hairpins and the like, that preclude, cause to be less efficient or inhibit hybridization to a complementary sequence. Template secondary structure can prevent priming with a standard PCR primer which is unable to destabilize the internal hybridization and thus is unable to hybridize to the primer complement. Using polynucleotide combinations of the disclosure, template secondary structure is dehybridized (or melted) and hybridization with the complementary template regions occurs under appropriate conditions.

A long PCR primer is able to resolve secondary structure in a target polynucleotide, but is not able to simultaneously provide either the specificity or sensitivity near the 3′ (priming) end of the primer. This is because for a long PCR primer a large portion is hybridized to the target polynucleotide and a mismatch near the 3′ end of the primer relative to the target polynucleotide will not be sufficient to reduce priming efficiency. As a result, a PCR product will still be synthesized despite the mismatch(s).

The polynucleotide combinations of the disclosure offer other advantages. For example, short PCR primers alone are useful for precise sequence hybridization to the target polynucleotide, but in order to achieve the high specificity of primer binding to a target polynucleotide that is desired for PCR, the highest possible annealing temperature is typically chosen. This annealing temperature is chosen based on the melting temperature of a given primer, and for a short primer that annealing temperature will be relatively low. A low annealing temperature, however, has the disadvantage of allowing for non-specific hybridization of the short primer to the target polynucleotide, resulting in non-specific PCR product formation. Based on the relatively low annealing temperature that must be used to allow a short PCR primer to anneal to its target polynucleotide, short primers form duplexes with a target polynucleotide that are typically unstable even when they are 100% complementary to the target polynucleotide region. Moreover, these duplexes are even more unstable when the primer is less than 100% complementary (i.e., at least one mismatch between the primer and the target polynucleotide region). The polynucleotide combination of the disclosure helps to overcome the instability problem associated with using a short PCR primer and permit highly specific binding to a desired target. For example, combinations of the disclosure are able to discriminate between target sequences that differ by as little as a single base.

For example, the polynucleotide combination design described herein allows for use of a short primer domain (f) through hybridization of the first domain (a) of the fixer polynucleotide (i.e., “first polynucleotide (1)”) to the target polynucleotide and hybridization of the second domain [c] of the fixer polynucleotide (i.e., “first polynucleotide (1)”) to the second domain [d] of the primer polynucleotide (i.e., “second polynucleotide (2)”), thereby giving the effective result of an apparent “longer” primer sequence. This longer and discontinuous hybridization in effect stabilizes binding between the first domain (f) of the second polynucleotide (2) even if this region is as small as eight bases, thereby increasing the efficiency of PCR. In another embodiment, the regions of the target polynucleotide that are complementary to the first domain of the first polynucleotide (1) and second polynucleotide (2) need not be directly adjacent. The present disclosure provides polynucleotide primers that comprise domain (b) in the second polynucleotide (2) and domain (e) in the first polynucleotide (1) that allow the first domain (a) of the first polynucleotide (1) and the first domain (f) of the second polynucleotide (2) to “search” for their complements in the target polynucleotide. This allows the distance between region (A) and region (F) in the target polynucleotide to vary substantially. The flexible linker domains cannot hybridize to a sequence in the first polynucleotide (1), the second polynucleotide (2) or the target polynucleotide under conditions in which domain (c) specifically hybridizes to domain (d).

### TERMS

As used herein, a “standard” or “conventional” PCR primer is one that hybridizes over its entire length to a polynucleotide.

The term “domain” as used herein refers to a contiguous sequence on a polynucleotide primer of the disclosure. The term “region” as used herein refers to a contiguous or non-contiguous sequence on a target or non-target polynucleotide. The term “target polynucleotide” as used herein refers to a polynucleotide from which extension by a polymerase is desired, or to which a polynucleotide primer combination of the disclosure is intended to hybridize. Thus, a “non-target polynucleotide” is a polynucleotide from which extension by a polymerase is not desired, or is less desirable than that of a target polynucleotide, or to which a polynucleotide primer combination of the disclosure is intended to hybridize with less specificity than a target polynucleotide.

“Appropriate conditions” as used herein refers to those conditions that are determined by one of ordinary skill in the art, and generally refer to nucleic acid hybridization conditions. One of skill in the art will understand that “appropriate conditions” with respect to hybridization depend on factors including but not limited to length of a polynucleotide, relative G+C content, salt concentration and hybridization temperature. Additional hybridization conditions are discussed herein below.

“Specifically hybridize” as used herein means that a polynucleotide will hybridize substantially or only with a specific nucleotide sequence or a group of specific nucleotide sequences under stringent hybridization conditions when the sequence is present in a complex mixture of DNA or RNA. Stringent hybridization conditions are described herein below. One or more nucleic acids are said to be “sufficiently complementary” when, given a certain set of hybridizing conditions, the one or more nucleic acids hybridize to each other. Accordingly, one or more nucleic acids are said to be “not sufficiently complementary” when, given a certain set of hybridizing conditions, the one or more nucleic acids do not hybridize to each other. “Fully complementary” or “perfectly complementary” as used herein means that a polynucleotide is 100% complementary to another polynucleotide.

A “mutation” as used herein refers to one or more nucleotides in a polynucleotide that differ from one or more corresponding nucleotides in a wild-type polynucleotide. Examples of a mutation include but are not limited to an insertion, a deletion, a substitution and an inversion. In various aspects of the disclosure, a mutation is in a target polynucleotide, with a wild-type sequecne being in a non-target polynucleotide.

As used herein, a “chemical polymer” sequence is all or in part a non-nucleic acid sequence that can be incorporated into a polynucleotide, but cannot hybridize to a nucleic acid. In one aspect, the chemical polymer is hydrophilic. Chemical polymers contemplated by the disclosure include but are not limited to polyethylene glycol (PEG), a peptide and a polysaccharide.

An “abasic site” as used herein is a Apurinic/Apyrimidinic (AP) site in a nucleic acid sequence or a chemical polymer that can be recognized and cleaved by an endonuclease.

A polynucleotide is said to “overlap” with another polynucleotide when one or more bases of each polynucleotide can hybridize to the same one or more bases of a target or non-target polynucleotide. By way of example, where a first polynucleotide is complementary to a region in a target nucleic acid and a second polynucleotide is complementary to all or part of the same region in the target polynucleotide, the first polynucleotide and the second polynucleotide are said to overlap.

A “unique polynucleotide sequence” as used herein refers to a sequence in a polynucleotide primer that is not complementary to a sequence in either a polynucleotide primer, a target polynucleotide or a non-target polynucleotide.

Percent complementarity or “% complementary” as used herein refers to a relative number of bases in a polynucleotide that are complementary to a number of bases in another polynucleotide. Thus, in one non-limiting example, if 18 out of 20 nucleotides in a polynucleotide primer of the disclosure are perfectly complementary to a target polynucleotide, the polynucleotide primer is said to be 90% complementary to the target polynucleotide.

It is noted here that, as used in this specification and the appended claims, the singular forms “a,” “an,” and “the” include plural reference unless the context clearly dictates otherwise.

### I. Polynucleotide Primer Combinations

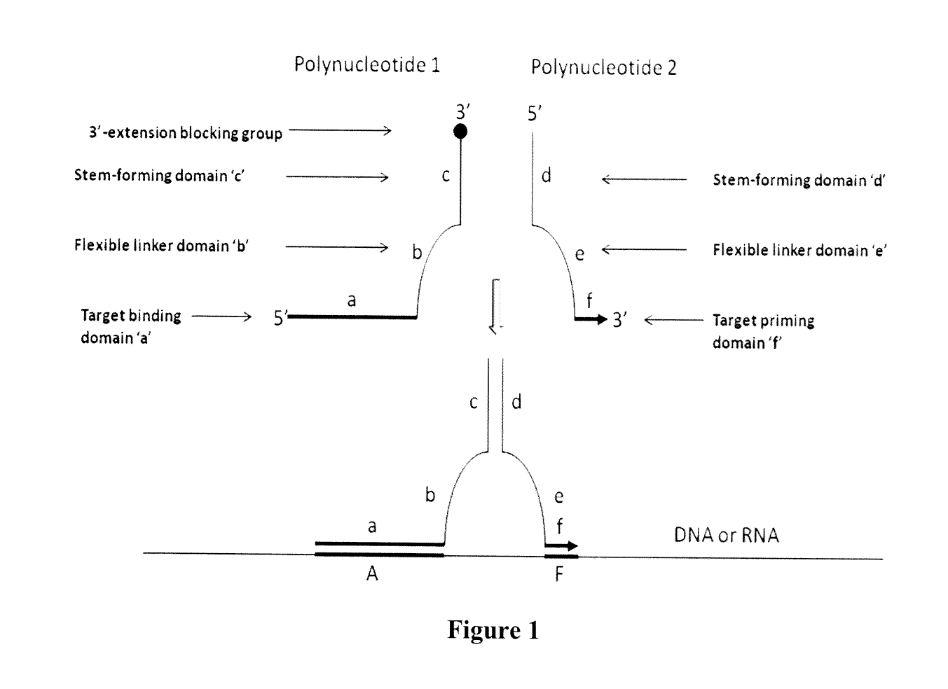

In one embodiment, the present disclosure provides a polynucleotide primer combination comprising a first polynucleotide (1) and a second polynucleotide (2), the first polynucleotide (1) comprising a first domain (a) having a sequence that is sufficiently complementary to a first target polynucleotide region (A), a second domain (c) comprising a unique polynucleotide sequence, and a third domain (b) comprising a polymer sequence that is not sufficiently complementary to hybridize to a domain in the first polynucleotide (1), a domain in the second polynucleotide (2), or a domain in the target polynucleotide, wherein domains in the first polynucleotide are arranged 5′-a-b-c-3′; the second polynucleotide (2) comprising a first domain (f) having a sequence that is sufficiently complementary to a sequence in a second target polynucleotide region (F), a second domain (d) comprising a polynucleotide sequence sufficiently complementary to (c) such that (c) and (d) will hybridize under appropriate conditions, and a third domain (e) comprising a polynucleotide sequence that is not sufficiently complementary to hybridize to a domain in the first polynucleotide (1), a domain in the second polynucleotide (2), or a domain in the target polynucleotide, wherein domains in the second polymer are arranged 5′-d-e-f-3′, wherein under conditions in which region (A) specifically hybridizes to domain (a) and region (F) specifically hybridizes to domain (f), domain (c) hybridizes to domain (d) and neither domain (b) nor domain (e) hybridizes to a domain in the first polynucleotide (1), a domain in second polynucleotide (2) or a domain in the target polynucleotide (see FIG. 1).

In some embodiments, the polymer sequence of domain (b) is selected from the group consisting of a polynucleotide sequence, a polynucleotide sequence comprising at least one modified nucleotide, a non-polynucleotide chemical polymer sequence and combinations thereof. In further embodiments, the polynucleotide sequence of domain (e) is selected from the group consisting of a polynucleotide sequence of naturally-occurring nucleotides and a polynucleotide sequence comprising at least one modified nucleotide, wherein the polynucleotide sequence of domain (e) is a template for polymerase extension.

In another embodiment, a polynucleotide primer combination is provided further comprising a domain (g) positioned between domain (d) and domain (e) in the second polynucleotide, wherein domain (g) comprises a replication blocker. In a further embodiment, a polynucleotide primer combination is provided wherein the second polynucleotide further comprises a fourth domain (h) positioned between domain (f) and domain (e), wherein domain (h) comprises a replication blocker. In various aspects, the replication blocker is selected from the group consisting of a modified base, an abasic site and a polymer.

In some aspects, a polynucleotide primer combination is provided wherein the second polynucleotide (2) further comprises a domain (s) positioned 5′ of domain (d), wherein domain (s) comprises a sequence that is not sufficiently complementary to the first polynucleotide (1) or the second polynucleotide (2) to hybridize to the first polynucleotide (1) or the second polynucleotide (2) under conditions in which domain (a) specifically hybridizes to region (A), and optionally wherein domain (s) is sufficiently complementary to a polynucleotide extended from domain (f) to hybridize to the polynucleotide extended from domain (f) under conditions in which domain (a) specifically hybridizes to region (A). In various embodiments, domain (s) further comprises a detectable marker, and in still further embodiments domain (s) further comprises a quencher that quenches the detectable marker.

In another embodiment, a polynucleotide primer combination is provided wherein domain (a) of the first polynucleotide (1) further comprises a domain (j), wherein domain (j) is contiguous with domain (a) and is positioned 3′ of domain (a), and wherein domain (j) is sufficiently complementary to a region (F*) in a non-target polynucleotide to hybridize under conditions in which domain (a) specifically hybridizes to region (A), wherein region (F*) in the non-target polynucleotide differs from region (F) in the target polynucleotide at at least one nucleotide. In a related embodiment, a polynucleotide primer combination is provided wherein domain (a) and domain (j) are not contiguous and are separated by a domain (p), wherein domain (p) is not sufficiently complementary to a domain in the first polynucleotide (1), a domain in the second polynucleotide (2), or the target polynucleotide to hybridize to the first polynucleotide (1), the second polynucleotide (2) or the target polynucleotide under conditions wherein domain (a) specifically hybridizes to region (A).

In some aspects, a polynucleotide primer combination is provided wherein domain (c) is at least 70% complementary to domain (d). In another embodiment, a polynucleotide primer combination is provided wherein domain (d) is at least 70% complementary to domain (c). With respect to domain (c) and domain (d), it is contemplated that in some aspects domain (d) and domain (c) are sufficiently complementary to hybridize to each other in the absence of the template polynucleotide.

In another embodiment, a polynucleotide primer combination is provided further comprising a blocking group attached to the first polynucleotide at its 3′ end which blocks extension from a DNA polymerase. In various aspects, the blocking group is selected from the group consisting of a 3′ phosphate group, a 3′ amino group, a dideoxy nucleotide, and an inverted deoxythymidine (dT).

With respect to the target polynucleotide, it is contemplated that in various embodiments the first target polynucleotide region (A) and the second target polynucleotide region (F) are separated by 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 10000, 20000, 30000, 40000, 50000, 60000, 70000, 80000, 90000 or 100000 or more bases in the target polynucleotide. In further aspects, the first target polynucleotide region (A) and the second target polynucleotide region (F) are separated by at least 110 kilobases, at least 120 kilobases, at least 130 kilobases, at least 140 kilobases, at least 150 kilobases, at least 160 kilobases, at least 170 kilobases, at least 180 kilobases, at least 190 kilobases, at least 200 kilobases, at least 210 kilobases, at least 220 kilobases, at least 230 kilobases, at least 240 kilobases, at least 250 kilobases, at least 260 kilobases, at least 270 kilobases, at least 280 kilobases, at least 290 kilobases, at least 300 kilobases, at least 310 kilobases, at least 320 kilobases, at least 330 kilobases, at least 340 kilobases, at least 350 kilobases, at least 360 kilobases, at least 370 kilobases, at least 380 kilobases, at least 390 kilobases, at least 400 kilobases, at least 410 kilobases, at least 420 kilobases, at least 430 kilobases, at least 440 kilobases, at least 450 kilobases, at least 460 kilobases, at least 470 kilobases, at least 480 kilobases, at least 490 kilobases, at least 500 kilobases, at least 510 kilobases, at least 520 kilobases, at least 530 kilobases, at least 540 kilobases, at least 550 kilobases, at least 560 kilobases, at least 570 kilobases, at least 580 kilobases, at least 590 kilobases, at least 600 kilobases, at least 610 kilobases, at least 620 kilobases, at least 630 kilobases, at least 640 kilobases, at least 650 kilobases, at least 660 kilobases, at least 670 kilobases, at least 680 kilobases, at least 690 kilobases, at least 700 kilobases, at least 710 kilobases, at least 720 kilobases, at least 730 kilobases, at least 740 kilobases, at least 750 kilobases, at least 760 kilobases, at least 770 kilobases, at least 780 kilobases, at least 790 kilobases, at least 800 kilobases, at least 810 kilobases, at least 820 kilobases, at least 830 kilobases, at least 840 kilobases, at least 850 kilobases, at least 860 kilobases, at least 870 kilobases, at least 880 kilobases, at least 890 kilobases, at least 900 kilobases, at least 910 kilobases, at least 920 kilobases, at least 930 kilobases, at least 940 kilobases, at least 950 kilobases, at least 960 kilobases, at least 970 kilobases, at least 980 kilobases, at least 990 kilobases, at least 1 megabase, at least 1.5 megabases, at least 2 megabases, at least 2.5 megabases, at least 3 megabases, at least 3.5 megabases, at least 4 megabases, at least 4.5 megabases, at least 5 megabases, at least 5.5 megabases, at least 6 megabases, at least 6.5 megabases, at least 7 megabases, at least 7.5 megabases, at least 8 megabases, at least 8.5 megabases, at least 9 megabases, at least 9.5 megabases, at least 10 megabases, at least 10.5 megabases, at least 11 megabases, at least 11.5 megabases, at least 12 megabases, at least 12.5 megabases, at least 13 megabases, at least 13.5 megabases, at least 14 megabases, at least 14.5 megabases, at least 15 megabases, at least 15.5 megabases, at least 16 megabases, at least 16.5 megabases, at least 17 megabases, at least 17.5 megabases, at least 18 megabases, at least 18.5 megabases, at least 19 megabases, at least 19.5 megabases, at least 20 megabases or more in the target polynucleotide.

In some aspects, a polynucleotide primer combination is provided wherein domain (f) is at least about 2, 3, 4, 5, 6, 7, 8, 9, 10, 11, 12, 13, 14, 15, 16, 17, 18, 19 or 20 bases in length. In one aspect, domain (f) is 8 bases in length. It is also contemplated that in some embodiments the sequence of domain (f) is 100% complementary to the sequence of the second target polynucleotide region (F). However, in certain aspects of the disclosure the sequence of domain (f) comprises a mismatch with the sequence of the non-target polynucleotide region (F*). In these aspects, the mismatch in the sequence of domain (f) with respect to the sequence of the second target polynucleotide region (F) is not a 3′ base mismatch, or the mismatch in the sequence of domain (f) with respect to the sequence of the second target polynucleotide region (F) is a 3′ base mismatch. In some aspects, the mismatch is a mutation.

Accordingly, polynucleotide primer combinations of the disclosure are useful in the highly specific detection of mutant sequences. In various aspects, the mutation is selected from the group consisting of an insertion, a deletion, a substitution and an inversion.

### A. Primer Combinations Comprising a Blocker Polynucleotide

The disclosure contemplates embodiments wherein a blocker polynucleotide is included with a polynucleotide combination. A blocker polynucleotide comprises a nucleotide sequence that is sufficiently complementary to all or part of region (F) such that the blocker polynucleotide will hybridize to all or part of region (F) under appropriate conditions. In some embodiments, the blocker polynucleotide overlaps with the first domain (f) of the second polynucleotide (2). In other words, the nucleotide(s) at the 3′ end of the second polynucleotide (2) and the nucleotide(s) at the 5′ end of the blocker polynucleotide would be complementary to the same nucleotide(s) of the target polynucleotide. In some aspects, the blocker polynucleotide has a sequence that overlaps (f) over the entire length of (f). In various embodiments, the overlap of the second polynucleotide (2) and the blocker polynucleotide is 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, or 15 nucleotides. In related embodiments, the nucleotide(s) at the 3′ end of the second polynucleotide (2) and the nucleotide(s) at the 5′ end of the blocker polynucleotide are different. In these embodiments, the nucleotide(s) at the 3′ end of the second polynucleotide (2) would hybridize to the target polynucleotide when they are complementary to the target polynucleotides at the appropriate position, thus allowing for extension of the second polynucleotide (2) under the appropriate conditions. In related embodiments, the nucleotide(s) at the 5′ end of the blocker polynucleotide would not hybridize to the target polynucleotide when the blocker polynucleotide is perfectly complementary to the non-target polynucleotide at the appropriate position, thus blocking extension of the second polynucleotide (2) from the non-target polynucleotide. In various embodiments, the nucleotide at the 3′ end of the blocker polynucleotide is modified to prevent extension by a polymerase.

In some embodiments, the overlapping sequences of the blocker polynucleotide and the first domain (f) of the second polynucleotide (2) differ by at least 2 bases, at least 3 bases, at least 4 bases, at least 5 bases, at least 6 bases, at least 7 bases, at least 8 bases, at 9 two bases, or by at least 10 bases. The differing bases can be at any position in the overlapping portions.

In further embodiments, the blocker polynucleotide is sufficiently complementary to hybridize under appropriate conditions to a third target polynucleotide region (X), wherein region (X) is between region (A) and region (F). In various aspects, region (X) comprises a sequence that is at least about 1 nucleotide to about 100 kilobases in length.

In some embodiments, a polynucleotide primer combination of the disclosure further comprises a first rigid frame blocker polynucleotide (RF1) and a second rigid frame blocker polynucleotide (RF2). Polynucleotide (RF1) comprises a domain (q) that is sufficiently complementary to a region (Xq) in a non-target polynucleotide to allow hybridization between domain (q) and region (Xq) under appropriate conditions, a domain (t) that is sufficiently complementary to a region (Xt) in the non-target polynucleotide to allow hybridization to allow hybridization between domain (t) and region (Xt), and a domain (r) that is sufficiently complementary to a domain (v) in polynucleotide (RF2) to allow hybridization between domain (r) and domain (v) under appropriate conditions; and wherein polynucleotide (RF2) comprises a domain (u) that is sufficiently complementary to a region (Xu) in a non-target polynucleotide to allow hybridization between domain (u) and region (Xu) under appropriate conditions, a domain (w) that is sufficiently complementary to a region (Xw) in the non-target polynucleotide to allow hybridization between domain (w) and region (Xw), and domain (v) that is sufficiently complementary to domain (r) in polynucleotide (RF 1) to allow hybridization between domain (r) and domain (v) under appropriate conditions, and wherein when domain (q) is specifically hybridized to region (Xq), and domain (t) is specifically hybridized to region (Xt), and domain (u) is specifically hybridized to region (Xu), and domain (w) is specifically hybridized to region (Xw), and domain (r) is specifically hybridized to domain (v), and domain (a) is specifically hybridized to region (A), domain (f) will not hybridize to region (F), and wherein region (Xq), region (Xt), region (Xu) and region (Xw) are not in the target polynucleotide.

In another aspect, the blocker polynucleotide is sufficiently complementary to hybridize under appropriate conditions to a fourth target polynucleotide region (Y), wherein region (Y) is 5′ of region (F).

In further aspects, the blocker polynucleotide is sufficiently complementary to hybridize under appropriate conditions to the entire length of region (F) and, in additional aspects, is able to hybridize under appropriate conditions either partially or entirely to region (A).

In some aspects, domain (b) of polynucleotide 1 is absent (i.e., comprises 0 bases), while in further aspects, domain (e) is absent (i.e., comprises 0 bases). In these aspects, it will be understood that in aspects wherein domain (b) is absent, domain (e) is present. Conversely, in aspects wherein domain (e) is absent, domain (b) is present. Accordingly, the disclosure contemplates that either domain (b) or domain (e) is absent in various aspects, but in no aspect are both domain (b) and domain (e) simultaneously absent.

The blocker polynucleotide is, in various aspects, from at least about 1 to at least about 100 bases or more in length. In one aspect, the blocker polynucleotide is 20 bases in length.

### B. Primer Combinations Comprising a Probe Polynucleotide

The disclosure also contemplates embodiments further comprising a probe polynucleotide. In some embodiments, a probe polynucleotide comprises a nucleotide sequence that is complementary to the fourth target polynucleotide region (Y). In other embodiments, a probe polynucleotide has a sequence that is complementary to the extension product of the second polynucleotide. As is apparent, this probe polynucleotide would be complementary to the complementary strand of the target polynucleotide. In embodiments wherein a blocker polynucleotide is included in the primer combination with the probe polynucleotide, the probe polynucleotide is complementary to a target polynucleotide region located 5′ of the target polynucleotide region complementary to the blocker polynucleotide. In various embodiments, the probe polynucleotide comprises a label at its 5′ end. In related embodiments, the probe polynucleotide further comprises a quencher at its 3′ end. In still further embodiments, the probe polynucleotide further comprises an internal quencher, such as, and without limitation, the Zen quencher.

### C. Primer Combinations—Positioning of Domain (a) and Domain (f)

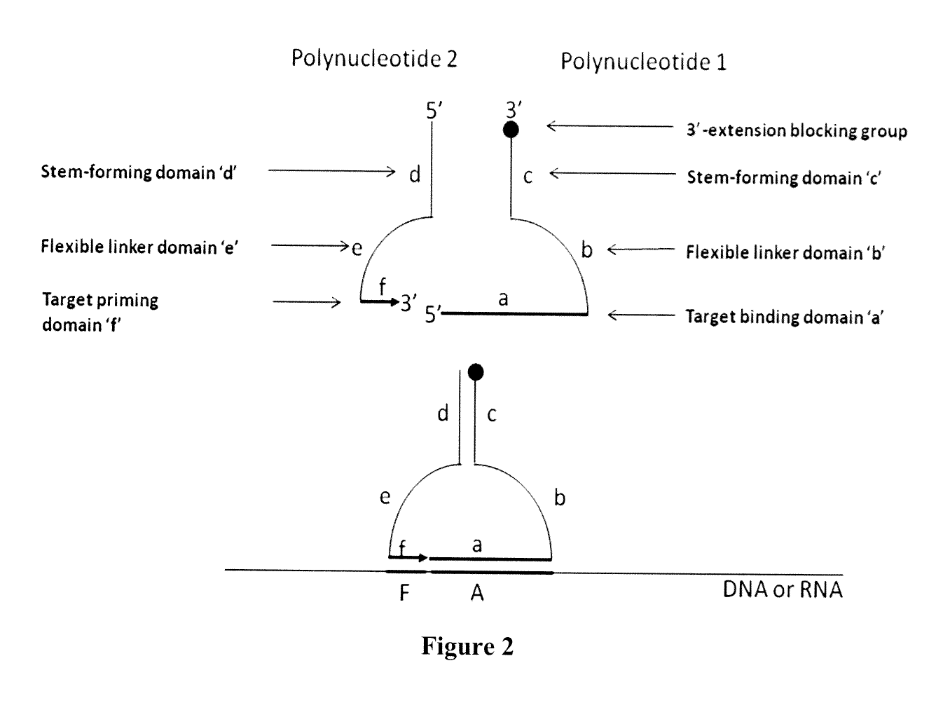

The disclosure provides polynucleotide primer combinations in which the relative positioning of domain (a) and domain (f) is inverted (see FIG. 2). Thus, in one aspect, a polynucleotide primer combination is provided wherein region (A) is 3′ to region (F) in the target polynucleotide. In another aspect, a polynucleotide primer combination is provided wherein region (A) is 5′ to region (F) in the target polynucleotide.

In aspects wherein region (A) is 5′ to region (F), it is contemplated that in some aspects, at least one nucleotide in domain (a) overlaps at least one nucleotide in domain (f) when each are hybridized to their respective region in the target polynucleotide.

In some aspects of the disclosure, domain (a) comprises a label, and in further aspects the label is quenchable. Thus, in some aspects domain (a) comprises a quencher, wherein the quencher is selected from the group consisting of Black Hole Quencher 1, Black Hole Quencher-2, Iowa Black FQ, Iowa Black RQ, a G-Base, and Dabcyl.

### D. Primer Combinations Comprising a Reverse Primer

The disclosure also contemplates embodiments wherein a reverse primer polynucleotide is included with the above polynucleotide combinations. The reverse primer is complementary to a region in the polynucleotide created by extension of the second polynucleotide. As is apparent, in some embodiments the reverse primer is also complementary to the complementary strand of the target polynucleotide when the target polynucleotide is one strand of a double-stranded polynucleotide. In some embodiments, the reverse primer is a combination first polynucleotide/second polynucleotide, as described above.

### II. Polynucleotides

As used herein, the term “polynucleotide,” either as a component of a polynucleotide pair combination, including blocker polynucleotides and probes, or as a target molecule, is used interchangeably with the term oligonucleotide and the term “nucleic acid.”

The term “nucleotide” or its plural as used herein includes naturally-occurring and modified forms as discussed herein and otherwise known in the art. In certain instances, the art uses the term “nucleobase” which embraces naturally-occurring nucleotides as well as modifications of nucleotides that can be polymerized.

Methods of making polynucleotides of a predetermined sequence are well-known in the art. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

In various aspects, methods provided include use of polynucleotides which are DNA oligonucleotides, RNA oligonucleotides, or combinations of the two types. Modified forms of oligonucleotides are also contemplated which include those having at least one modified internucleotide linkage. Modified polynucleotide also include a chemical polymer as used herein. Modified polynucleotides or oligonucleotides are described in detail herein below.

### III. Modified Polynucleotides

Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.” In specific embodiments, the first polynucleotide comprises phosphorothioate linkages.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still other embodiments, a modified oligonucleotide includes mimetics wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., 1991, Science, 254: 1497-1500, the disclosures of which are herein incorporated by reference.

In still other embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligo consists of 2 to 4, desirably 3, groups/atoms selected from —CH₂, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}—P(O)₂—O—, —O—P(O,NR^{H})—O—, —CH₂—P(O)₂—O—, O—P(O)₂—CH₂—, and —O—Si(R″)₂—O—; among which —CH₂—CO—NR^{H}—, CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of oligonucleotides are described in detail in U.S. patent application NO. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₘCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples herein below.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In various aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226, the disclosures of which are incorporated by reference in their entireties herein. In various embodiments, the first polynucleotide comprises a locked nucleic acid. In some embodiments, the first polynucleotide comprises a plurality of locked nucleic acids. In specific embodiments, the first domain of the first polynucleotide comprises a plurality of locked nucleic acids. In more specific embodiments, the nucleotide at the 3′ end of the first polynucleotide comprises a locked nucleic acid. In various embodiments, the blocker polynucleotide comprises a locked nucleic acid. In other embodiments, the blocker polynucleotide comprises a plurality of locked nucleic acids. In specific embodiments, the nucleotide at the 5′ end of the blocker polynucleotide comprises a locked nucleic acid.

Polynucleotides may also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

A “modified base” or other similar term refers to a composition which can pair with a natural base (e.g., adenine, guanine, cytosine, uracil, and/or thymine) and/or can pair with a non-naturally occurring base. In certain aspects, the modified base provides a Tₘ differential of 15, 12, 10, 8, 6, 4, or 2° C. or less. Exemplary modified bases are described in EP 1 072 679 and WO 97/12896.

By “nucleobase” is meant the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N′,N′-ethano-2,6-diaminopurine, 5-methylcytosine (mC), 5-(C³-C⁶)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). The term “nucleosidic base” or “base unit” is further intended to include compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

### IV. Polynucleotide Structure—Length

In one embodiment, the first polynucleotide (1) comprises a first domain (a) containing about 10 nucleotides, this first domain (a) of the first polynucleotide (1) being complementary to a target polynucleotide region that is different from the target region recognized by the first domain (f) of the second polynucleotide (2). In various aspects, the first polynucleotide (1) comprises a first domain (a) containing at least 11, at least 12, at least 13, at least 14, at least 15, at least 16, at least 17, at least 18, 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, at least about 2600, at least about 2700, at least about 2800, at least about 2900, at least about 3000, at least about 3100, at least about 3200, at least about 3300, at least about 3400, at least about 3500, at least about 3600, at least about 3700, at least about 3800, at least about 3900, at least about 4000, at least about 4100, at least about 4200, at least about 4300, at least about 4400, at least about 4500, at least about 4600, at least about 4700, at least about 4800, at least about 4900, at least about 5000 or more nucleotides, the first domain (a) of this first polynucleotide (1) being complementary, or sufficiently complementary, so as to recognize and bind to a target polynucleotide region that is different from the target region recognized by the first domain (f) of the second polynucleotide (2). In a related aspect, the second domain (d) of the second polynucleotide (2) comprises 6 or more nucleotides in a unique DNA polynucleotide sequence that is sufficiently complementary to the second domain (c) of the first polynucleotide (1) so as to allow hybridization between these two complementary sequences under appropriate conditions. In a related aspect, the second domain (c) of the first polynucleotide (1) comprises 6 or more nucleotides in a unique polynucleotide sequence that is sufficiently complementary to the second domain (d) of the second polynucleotide (2) so as to allow hybridization between these two complementary sequences under appropriate conditions. In various aspects, the second domain (c) of the first polynucleotide (1) comprises at least 11, at least 12, at least 13, at least 14, at least 15, at least 16, at least 17, at least 18, at least 19, at least 20, at least 21, at least 22, at least 23, at least 24 nucleotides, at least 25, at least about 30, at least about 35, at least about 40, at least about 45, at least about 50, at least about 60, at least about 70, at least about 80, at least about 90, at least about 100, at least about 120, at least about 140, at least about 160, at least about 180, at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500 or more nucleotides of a unique polynucleotide sequence that is sufficiently complementary to the second domain (d) of the second polynucleotide (2) so as to allow hybridization between the two complementary sequences under appropriate conditions.

In another aspect, the first domain (f) of the second polynucleotide (2) is 2 nucleotides that are complementary to a target polynucleotide region (F). In various aspects, the first domain (f) of the second polynucleotide (2) is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides or more that is complementary to a target polynucleotide region (F). In a related aspect, the second domain (d) of the second polynucleotide (2) comprises 10 or more nucleotides in a unique polynucleotide sequence that is sufficiently complementary to the second domain (c) of the first polynucleotide (1) so as to allow hybridization between these two complementary sequences under appropriate conditions. In various aspects, the second domain (d) of the second polynucleotide (2) comprises at least 11, at least 12, at least 13, at least 14, at least 15, at least 16, at least 17, at least 18, at least 19, at least 20, at least 21, at least 22, at least 23, at least 24 nucleotides, at least 25, at least about 30, at least about 35, at least about 40, at least about 45, at least about 50, at least about 60, at least about 70, at least about 80, at least about 90, at least about 100, at least about 120, at least about 140, at least about 160, at least about 180, at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500 or more nucleotides of a unique polynucleotide sequence that is sufficiently complementary to the second domain (c) of the first polynucleotide (1) so as to allow hybridization between the two complementary sequences under appropriate conditions.

As described herein, the third domain (b) of the first polynucleotide (1) comprises a polymer sequence is selected from the group consisting of a polynucleotide sequence, a polynucleotide sequence comprising at least one modified nucleotide, and a non-polynucleotide chemical polymer sequence. In aspects wherein domain (b) is a polynucleotide sequence, the polynucleotide sequence is, in various aspects, at least 1 nucleotide in length. Similarly, domain (e) of the second polynucleotide is, in some aspects, at least 1 nucleotide in length and can comprise a polynucleotide sequence of naturally-occurring nucleotides and a polynucleotide sequence comprising at least one modified nucleotide. In further aspects, domain (b) of the first polynucleotide (1) and/or domain (e) of the second polynucleotide (2) are at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 nucleotides, at least 25, at least about 30, at least about 35, at least about 40, at least about 45, at least about 50, at least about 60, at least about 70, at least about 80, at least about 90, at least about 100, at least about 120, at least about 140, at least about 160, at least about 180, at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500 or more nucleotides in length.

In some embodiments, compositions and methods described herein include a second set of polynucleotides with the characteristics described above for first and second polynucleotides. In some embodiments, a plurality of sets is contemplated. These additional sets of first and second polynucleotides can have any of the characteristics described for first and second polynucleotides.

The rigid frame blocker polynucleotides are contemplated in one aspect to comprise at least 30 nucleotides. In other aspects, the rigid frame blocker polynucleotides can comprise at least 31 nucleotides, or at least 32 nucleotides, or at least 33 nucleotides, or at least 34 nucleotides, or at least 35 nucleotides, or at least 36 nucleotides, or at least 37 nucleotides, or at least 38 nucleotides, or at least 39 nucleotides, or at least 40 nucleotides, or at least about 45 nucleotides, or at least about 50 nucleotides, or at least about 55 nucleotides, or at least about 60 nucleotides, or at least about 65 nucleotides, or at least about 70 nucleotides, or at least about 75 nucleotides, or at least about 80 nucleotides, or at least about 85 nucleotides, or at least about 90 nucleotides, or at least about 95 nucleotides, or at least about 100 nucleotides, or at least about 105 nucleotides, or at least about 110 nucleotides, or at least about 150 nucleotides, or at least about 200 nucleotides, or at least about 250 nucleotides, or at least about 300 nucleotides, or at least about 350 nucleotides, or at least about 400 nucleotides, or at least about 450 nucleotides, or at least 500 nucleotides or more.

In some embodiments, the probe polynucleotide is from about 5 nucleotides in length to about 100 bases in length. In various aspects, the probe polynucleotide comprises at least 5 nucleotides, or at least 6 nucleotides, or at least 7 nucleotides, or at least 8 nucleotides, or at least 9 nucleotides, or at least 10 nucleotides, or at least 11 nucleotides, or at least 12 nucleotides, or at least 13 nucleotides, or at least 14 nucleotides, or at least 15 nucleotides, or at least 16 nucleotides, or at least 17 nucleotides, or at least 18 nucleotides, or at least 19 nucleotides, or at least 20 nucleotides, or at least 21 nucleotides, or at least 22 nucleotides, or at least 23 nucleotides, or at least 24 nucleotides, or at least 25 nucleotides, or at least 26 nucleotides, or at least 27 nucleotides, or at least 28 nucleotides, or at least 29 nucleotides, or at least 30 nucleotides, or at least 31 nucleotides, or at least 32 nucleotides, or at least 33 nucleotides, or at least 34 nucleotides, or at least 35 nucleotides, or at least 36 nucleotides, or at least 37 nucleotides, or at least 38 nucleotides, or at least 39 nucleotides, or at least 40 nucleotides, or at least about 45 nucleotides, or at least about 50 nucleotides, or at least about 55 nucleotides, or at least about 60 nucleotides, or at least about 65 nucleotides, or at least about 70 nucleotides, or at least about 75 nucleotides, or at least about 80 nucleotides, or at least about 85 nucleotides, or at least about 90 nucleotides, or at least about 95 nucleotides, or at least about 100 nucleotides of a DNA sequence that is sufficiently complementary to a target polynucleotide region so as to allow hybridization under appropriate conditions.

In some embodiments, the blocker polynucleotide is from about 5 nucleotides in length to about 1000 bases in length. In various aspects, the blocker polynucleotide comprises at least 5 nucleotides, or at least 6 nucleotides, or at least 7 nucleotides, or at least 8 nucleotides, or at least 9 nucleotides, or at least 10 nucleotides, or at least 11 nucleotides, or at least 12 nucleotides, or at least 13 nucleotides, or at least 14 nucleotides, or at least 15 nucleotides, or at least 16 nucleotides, or at least 17 nucleotides, or at least 18 nucleotides, or at least 19 nucleotides, or at least 20 nucleotides, or at least 21 nucleotides, or at least 22 nucleotides, or at least 23 nucleotides, or at least 24 nucleotides, or at least 25 nucleotides, or at least 26 nucleotides, or at least 27 nucleotides, or at least 28 nucleotides, or at least 29 nucleotides, or at least 30 nucleotides, or at least 31 nucleotides, or at least 32 nucleotides, or at least 33 nucleotides, or at least 34 nucleotides, or at least 35 nucleotides, or at least 36 nucleotides, or at least 37 nucleotides, or at least 38 nucleotides, or at least 39 nucleotides, or at least 40 nucleotides, or at least about 45 nucleotides, or at least about 50 nucleotides, or at least about 55 nucleotides, or at least about 60 nucleotides, or at least about 65 nucleotides, or at least about 70 nucleotides, or at least about 75 nucleotides, or at least about 80 nucleotides, or at least about 85 nucleotides, or at least about 90 nucleotides, or at least about 95 nucleotides, or at least about 100 nucleotides, or at least about 110 nucleotides, or at least about 120 nucleotides, or at least about 130 nucleotides, or at least about 140 nucleotides, or at least about 150 nucleotides, or at least about 160 nucleotides, or at least about 170 nucleotides, or at least about 180 nucleotides, or at least about 190 nucleotides, or at least about 200 nucleotides, or at least about 250 nucleotides, or at least about 300 nucleotides, or at least about 350 nucleotides, or at least about 400 nucleotides, or at least about 450 nucleotides, or at least about 500 nucleotides, or at least about 550 nucleotides, or at least about 600 nucleotides, or at least about 650 nucleotides, or at least about 700 nucleotides, or at least about 750 nucleotides, or at least about 800 nucleotides, or at least about 850 nucleotides, or at least about 900 nucleotides, or at least about 950 nucleotides, or at least about 1000 nucleotides of a polynucleotide sequence that is sufficiently complementary to a target polynucleotide region so as to allow hybridization under appropriate conditions. In various embodiments, the blocker polynucleotide further comprises a modified nucleotide, which in various aspects is an internal nucleotide and/or is the nucleotide at its 5′ end. In various embodiments, the modified nucleotide is a locked nucleic acid. In some embodiments, the blocker polynucleotide further comprises a blocking group at the 3′ end to prevent extension by a polymerase.

In some embodiments, the reverse primer polynucleotide is from about 5 nucleotides in length to about 100 bases in length. In various aspects, the reverse primer polynucleotide comprises at least 5 nucleotides, or at least 6 nucleotides, or at least 7 nucleotides, or at least 8 nucleotides, or at least 9 nucleotides, or at least 10 nucleotides, or at least 11 nucleotides, or at least 12 nucleotides, or at least 13 nucleotides, or at least 14 nucleotides, or at least 15 nucleotides, or at least 16 nucleotides, or at least 17 nucleotides, or at least 18 nucleotides, or at least 19 nucleotides, or at least 20 nucleotides, or at least 21 nucleotides, or at least 22 nucleotides, or at least 23 nucleotides, or at least 24 nucleotides, or at least 25 nucleotides, or at least 26 nucleotides, or at least 27 nucleotides, or at least 28 nucleotides, or at least 29 nucleotides, or at least 30 nucleotides, or at least 31 nucleotides, or at least 32 nucleotides, or at least 33 nucleotides, or at least 34 nucleotides, or at least 35 nucleotides, or at least 36 nucleotides, or at least 37 nucleotides, or at least 38 nucleotides, or at least 39 nucleotides, or at least 40 nucleotides, or at least about 45 nucleotides, or at least about 50 nucleotides, or at least about 55 nucleotides, or at least about 60 nucleotides, or at least about 65 nucleotides, or at least about 70 nucleotides, or at least about 75 nucleotides, or at least about 80 nucleotides, or at least about 85 nucleotides, or at least about 90 nucleotides, or at least about 95 nucleotides, or at least about 100 nucleotides of a polynucleotide sequence that is sufficiently complementary to a region of a polymerase-extended first polynucleotide so as to allow hybridization under appropriate conditions. In some embodiments, when the target polynucleotide is a double-stranded polynucleotide, the reverse primer is complementary to a complementary strand of the target polynucleotide. In some embodiments, the reverse primer is a combination of first and second polynucleotides, as defined herein.

### V. Polynucleotide Base Structure

In some embodiments, the first polynucleotide is comprised of DNA, modified DNA, RNA, modified RNA, PNA, or combinations thereof. In other embodiments, the second polynucleotide is comprised of DNA, modified DNA, RNA, modified RNA, PNA, or combinations thereof.

Vi. Polynucleotide Structure—Replication/Extension Blockers

An internal “replication blocker” is incorporated as needed when termination of polymerase extension of a polynucleotide is desirable. Similarly, 3′ terminal extension blockers are also contemplated by the disclosure. An “extension blocking group” or “extension blocker” is a moiety that prevents initiation of extension from the 3′ end of a polynucleotide. For example, the second domain (c) of the first polynucleotide (1), in another aspect, further comprises an extension blocker at the 3′ end of the second domain (c) to prevent extension by an enzyme that is capable of synthesizing a nucleic acid. In further aspects, the blocker polynucleotide comprises an extension blocker at its 3′ end. In additional aspects, domain (g), which is positioned between domain (d) and domain (e) in the second polynucleotide, comprises a replication blocker. In still further aspects, the second polynucleotide further comprises a fourth domain (h) positioned between domain (f) and domain (e), wherein domain (h) comprises a replication blocker.

Terminal 3′ extension blocking groups useful in the practice of the methods include but are not limited to a 3′ phosphate group, a 3′ amino group, a dideoxy nucleotide, and inverted deoxythymidine (dT). Internal replication blocking groups useful in the practice of the methods include but are not limited to an abasic site(s), a modified base(s), or a six carbon glycol spacer (and in one aspect the six carbon glycol spacer is hexanediol).

### VII. Polynucleotide Structure—Complementarity

In some aspects, the second domain (d) of the second polynucleotide (2) is at least about 70% complementary to the second domain (c) of the first polynucleotide (1). In related aspects, the second domain (d) of the second polynucleotide (2) is at least about 75%, at least about 80%, at least about 85%, at least about 90%, at least about 95%, or about 100% complementary to the second domain (c) of the first polynucleotide (1).

In another aspect, the blocker polynucleotide is at least about 75%, at least about 80%, at least about 85%, at least about 90%, at least about 95%, or about 100% complementary to a sequence in the target polynucleotide, and in yet another aspect, the probe polynucleotide is at least about 75%, at least about 80%, at least about 85%, at least about 90%, at least about 95%, or about 100% complementary to a sequence in the target polynucleotide, and/or a sequence in the blocker polynucleotide.

### VIII. Hybridization Conditions

In some embodiments, the first (1) and second polynucleotide (2) hybridize to each other under stringent conditions in the absence of a template polynucleotide. In some embodiments, the first (1) and second polynucleotides (2) do not hybridize to each other under stringent conditions in the absence of a template polynucleotide. “Stringent conditions” as used herein can be determined empirically by the worker of ordinary skill in the art and will vary based on, e.g., the length of the primer, complementarity of the primer, concentration of the primer, the salt concentration (i.e., ionic strength) in the hybridization buffer, the temperature at which the hybridization is carried out, length of time that hybridization is carried out, and presence of factors that affect surface charge of the polynucleotides. In general, stringent conditions are those in which the polynucleotide is able to bind to its complementary sequence preferentially and with higher affinity relative to any other region on the target. Exemplary stringent conditions for hybridization to its complement of a polynucleotide sequence having 20 bases include without limitation about 50% G+C content, 50 mM salt (Na⁺), and an annealing temperature of 60° C. For a longer sequence, specific hybridization is achieved at higher temperature. In general, stringent conditions are such that annealing is carried out about 5° C. below the melting temperature of the polynucleotide. The “melting temperature” is the temperature at which 50% of polynucleotides that are complementary to a target polynucleotide in equilibrium at definite ion strength, pH and polynucleotide concentration.

### IX. Methods of Use

### A. PCR

In target polynucleotide amplification methods described herein, a third polynucleotide (3) and a fourth polynucleotide (4) are contemplated for use in combination with the polynucleotide combination described above, the third polynucleotide (3) comprising a first domain (a) that is complementary to a complementary strand of the target polynucleotide [relative to the strand to which the first domain of the first polynucleotide (1) is complementary] at a first target complement polynucleotide region and a second domain (c) comprising a unique polynucleotide sequence, and the fourth polynucleotide (4) comprising a first domain (f) that is complementary to the complementary strand of the target polynucleotide [relative to the strand to which the second polynucleotide is complementary] at a second complement target polynucleotide region and a second domain (d) comprising a polynucleotide sequence sufficiently complementary to the second domain (c) of the third polynucleotide (3) such that the second domain (c) of the third polynucleotide (3) and the second domain (d) of the fourth polynucleotide (4) will hybridize under appropriate conditions. In some of these aspects, the method further comprises contacting the target polynucleotide and a complement of the target polynucleotide with the first polynucleotide (1) and second polynucleotide (2) and the third polynucleotide (3) and fourth polynucleotide (4) under conditions sufficient to allow hybridization of the first domain (a) of the first polynucleotide (1) to the first target polynucleotide region (A) of the target polynucleotide, the first domain (f) of the second polynucleotide (2) to the second target polynucleotide region (F) of the target polynucleotide, the first domain (a) of the third polynucleotide (3) to the first target domain of the complementary strand of the target polynucleotide and the first domain (f) of the fourth polynucleotide (4) to the second complement target polynucleotide region and extending the first domains (i.e., priming domains) of the second and fourth polynucleotides with a DNA polymerase under conditions which permit extension of the second polynucleotide and the fourth polynucleotide. In various embodiments, the third polynucleotide further comprises a third domain (b) comprising a polymer sequence that is not sufficiently complementary to hybridize to a domain in the first polynucleotide (1), a domain in the second polynucleotide (2), a domain in the third polynucleotide (3), a domain in the fourth polynucleotide (4) or a region in the target polynucleotide, wherein domains in the third polynucleotide are arranged 5′-a-b-c-3′. In related embodiments, the fourth polynucleotide further comprises a third domain (e) comprising a polynucleotide sequence that is not sufficiently complementary to hybridize to a domain in the first polynucleotide (1), a domain in the second polynucleotide (2), a domain in the third polynucleotide (3), a domain in the fourth polynucleotide (4) or a region in the target polynucleotide, wherein domains in the second polymer are arranged 5′-d-e-f-3′, wherein under conditions in which region (A) in the target polynucleotide specifically hybridizes to domain (a) and region (F) of the target polynucleotide specifically hybridizes to domain (f), domain (c) of the third polynucleotide (3) hybridizes to domain (d) of the fourth polynucleotide (4) and neither domain (b) nor domain (e) hybridizes to a domain in the first polynucleotide (1), a domain in second polynucleotide (2), a domain in the third polynucleotide (3), a domain in the fourth polynucleotide (4) or a region in the target polynucleotide.

In some aspects, an extension blocker as described herein above is attached to the first polynucleotide (1) and/or the third polynucleotide (3) at their 3′ ends which blocks extension by an enzyme that is capable of synthesizing a nucleic acid. Blocking groups useful in the practice of the methods include but are not limited to a 3′ phosphate group, a 3′ amino group, a dideoxy nucleotide, and inverted deoxythymidine (dT).

In various embodiments, the target polynucleotide, the complement of the target polynucleotide or both has a secondary structure that is denatured by hybridization of the first domain (a) of the first polynucleotide and/or the first domain (a) of the third polynucleotide to a target polynucleotide.

One of ordinary skill in the art will recognize that the polynucleotides of combinations of the present disclosure can be used to prime either one or both ends of a given PCR amplicon. As used herein, an “amplicon” is understood to mean a portion of a polynucleotide that has been synthesized using amplification techniques. It is contemplated that any of the methods of the present disclosure that comprise more than one polynucleotide combination may utilize any combination of standard primer and polynucleotide combination, provided at least one of the primers is a polynucleotide combination as described herein.

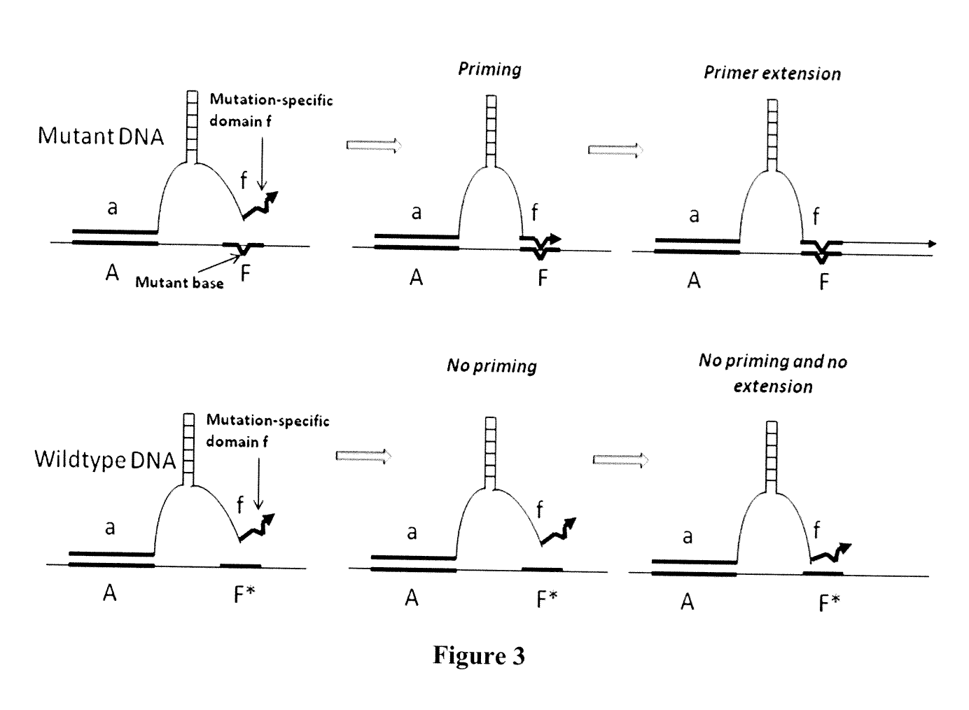

In various embodiments and as described herein, a polynucleotide primer combination of the disclosure is used to detect a mutation in a target polynucleotide versus a non-target polynucleotide. Accordingly, in some embodiments a polynucleotide primer combination is used to detect a polymorphism in a target polynucleotide (see FIG. 3). In FIG. 3, the polynucleotide sequence of domain (f) specifically hybridizes to a mutant DNA sequence (region (F)), but comprises at least one mismatch with a wildtype DNA sequence (region (F*)). Thus, region (f) can specifically hybridize to region (F) and serve to prime an extension product from the mutant DNA target polynucleotide.

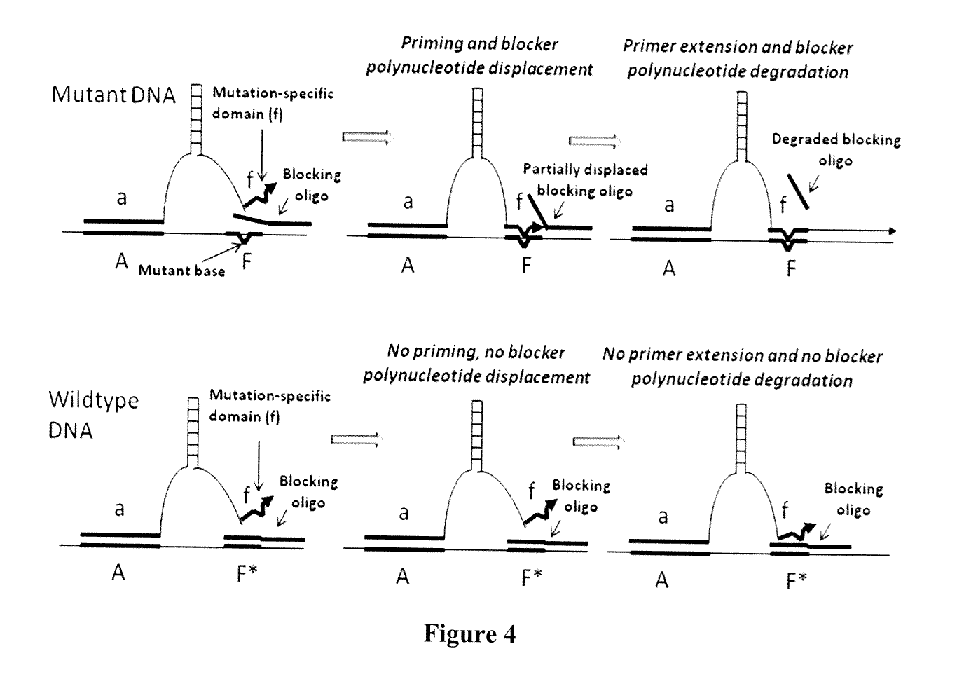

In related embodiments, a polynucleotide primer combination of the disclosure that is used to detect a mutation in a target polynucleotide versus a non-target polynucleotide further comprises a blocker polynucleotide (FIG. 4). FIG. 4 depicts target polynucleotide discrimination with a polynucleotide primer combination of the disclosure and a blocker polynucleotide. In some aspects, and as shown in FIG. 4, the blocker polynucleotide is degraded following displacement from the target polynucleotide. Specifically, in the presence of a target polynucleotide, mutation-specific domain (f) first invades region (F) and displaces the imperfectly annealed 5′ tail of the blocker polynucleotide. Then DNA polymerase with 5′ exonuclease activity (for example and without limitation, wildtype Tag DNA polymerase) cleaves the 5′ flap of the blocker polynucleotide. At the next step DNA polymerase replicates the DNA template in a nick-translation mode until the blocking oligonucleotide becomes too short to maintain a stable interaction with DNA and dissociates from the template allowing polymerase to proceed in normal replication mode. In the case of wildtype DNA (FIG. 4), mutation-specific domain (f) is unable to invade and displace the perfectly annealed blocker polynucleotide, and thus cannot prime DNA synthesis.

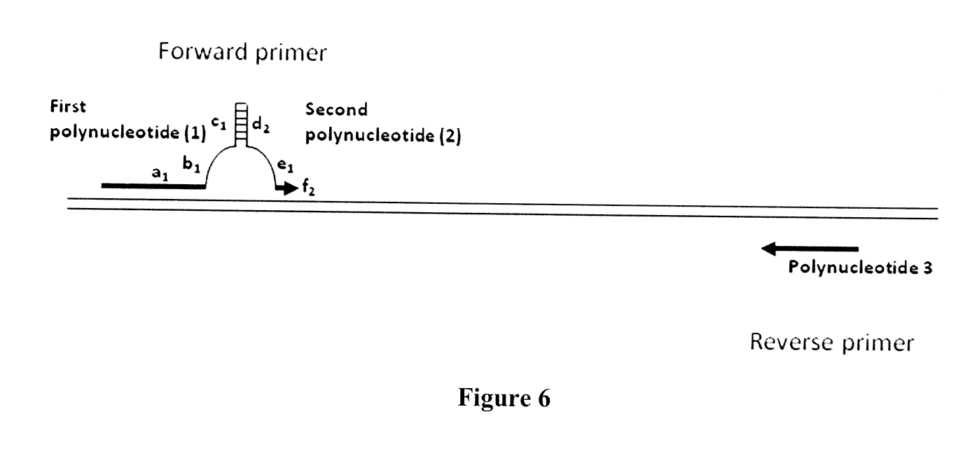

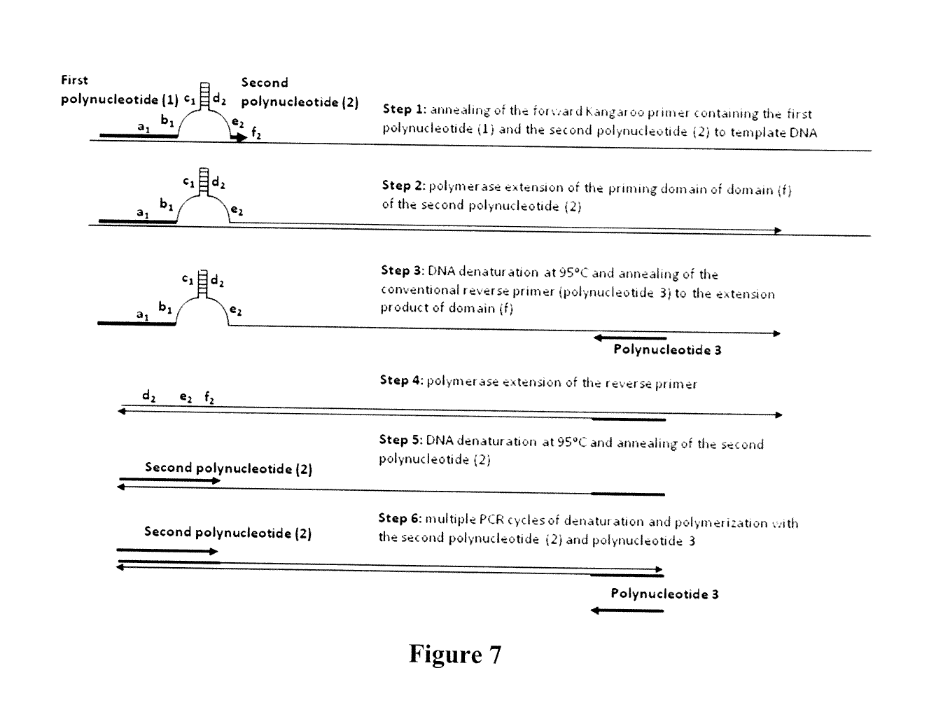

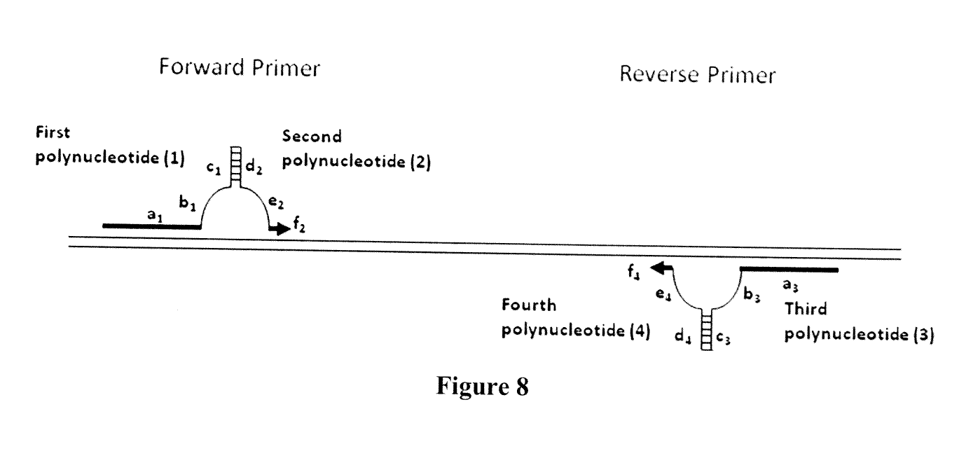

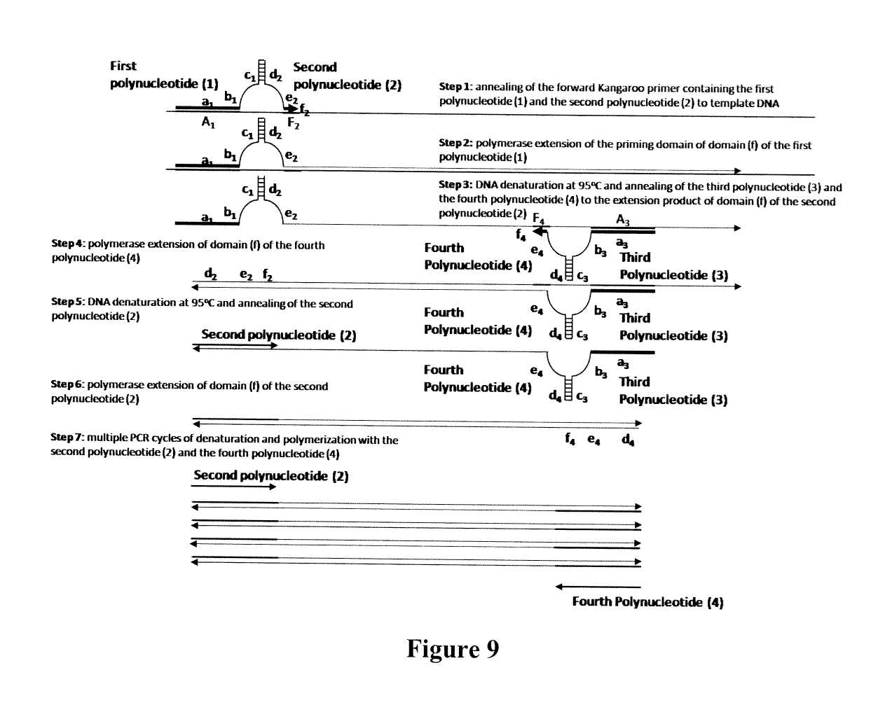

In some embodiments, a primer extension reaction and polymerase chain reactions as depicted in FIGS. 6-9 with one or two polynucleotide primer combinations of the disclosure are shown. Reactions depicted in FIGS. 6-9 are, in various aspects, executed using real-time PCR instruments (quantitative PCR) in the presence of staining dyes, which for example and without limitation include SybrGreen, or in the presence of probes, for example and without limitation TaqMan, beacon and Scorpions. In some aspects, the reactions use mutation-specific polynucleotide primers of the disclosure as described in FIG. 3. In further aspects, the reactions use blocker polynucleotides as described in FIG. 4.

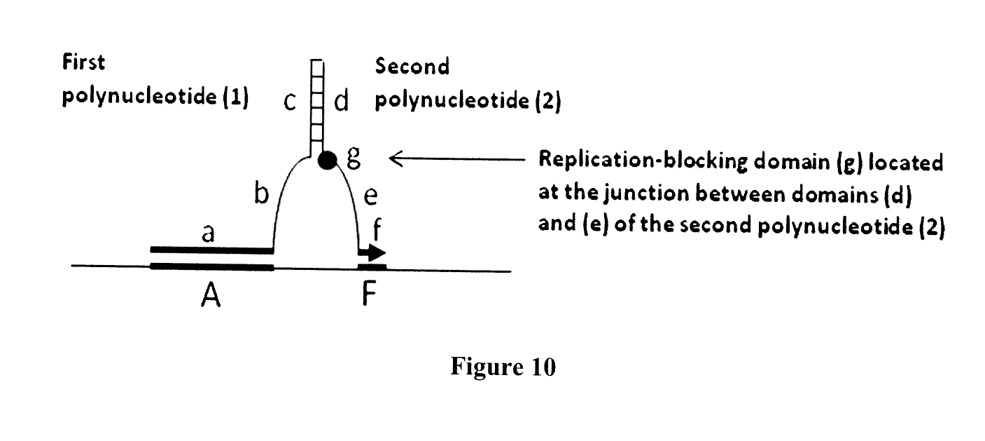

Also provided by the disclosure are embodiments wherein a polynucleotide primer combination of the disclosure further comprises a replication blocking domain (g) (FIG. 10). In this case the polynucleotide primer combination has an additional element, specifically a replication blocking domain (g) located between domains (d) and (e) of the second polynucleotide (2). Domain (g) can, in various aspects, be a modified base or linker or a polymer that would not allow a DNA polymerase to replicate domain (d) after finishing replication of domain (e) but rather would terminate replication at domain (g). In some aspects, the purpose of domain (g) is to eliminate sequence (c) of the first polynucleotide (1) from the 3′ end of the PCR product.

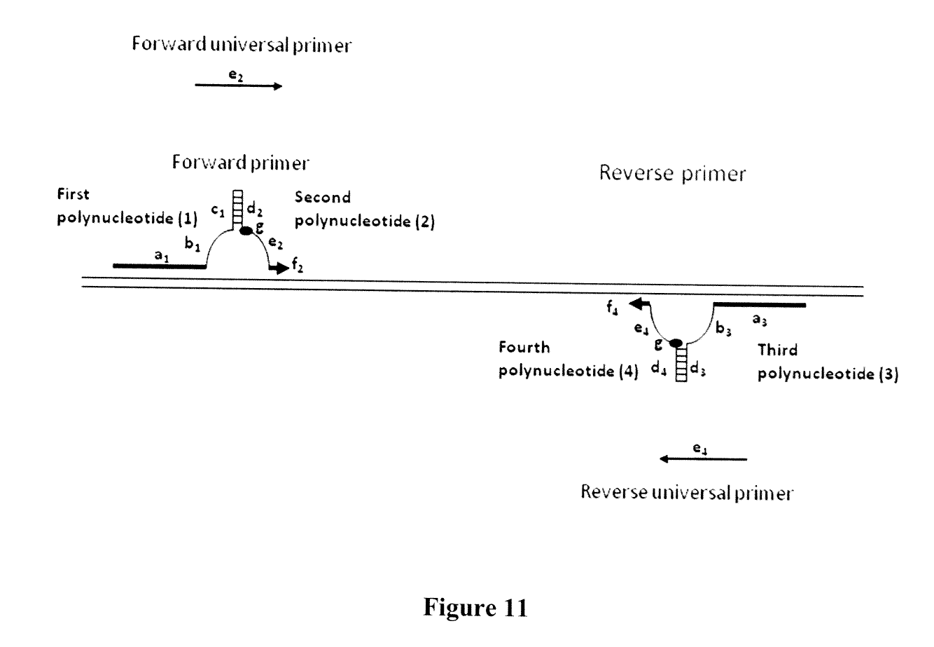

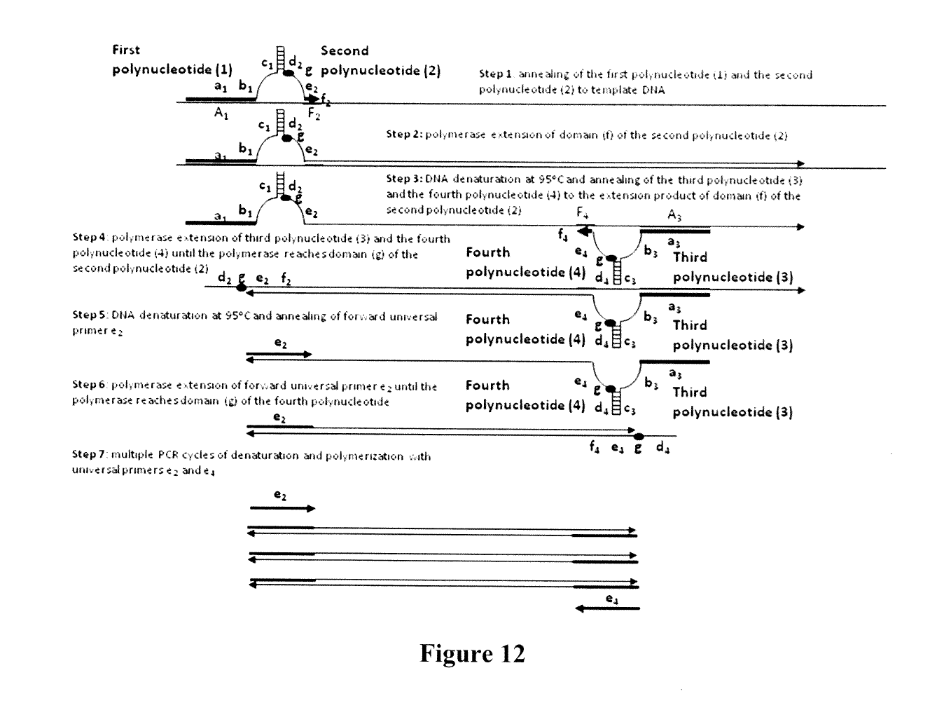

In additional embodiments, a polymerase chain reaction with two polynucleotide primer combinations of the disclosure, each comprising a replication-blocking domain (g) and two universal primers (e₂ and e₄ in FIGS. 11 and 12) that have no complementarity to the target polynucleotide. In some aspects, all four primers are present in the reaction tube from the start. During the first four steps, the first polynucleotide (1), second polynucleotide (2), third polynucleotide (3) and fourth polynucleotide (4) form a PCR amplicon. During the next PCR cycles, amplification can be supported by either the second polynucleotide (2) and the fourth polynucleotide (4) or by universal primers e₂ and e₄, or by all 4 primers (FIG. 12). If the concentration of the second polynucleotide (2) and the fourth polynucleotide (4) is substantially lower than the concentration of universal primers e₂ and e₄, or primers e₂ and e₄ have a higher stability than the second polynucleotide (2) and the fourth polynucleotide (4) due to introduced base modifications (for example and without limitation, LNAs) the amplification will be mostly accomplished by universal primers e₂ and e₄.

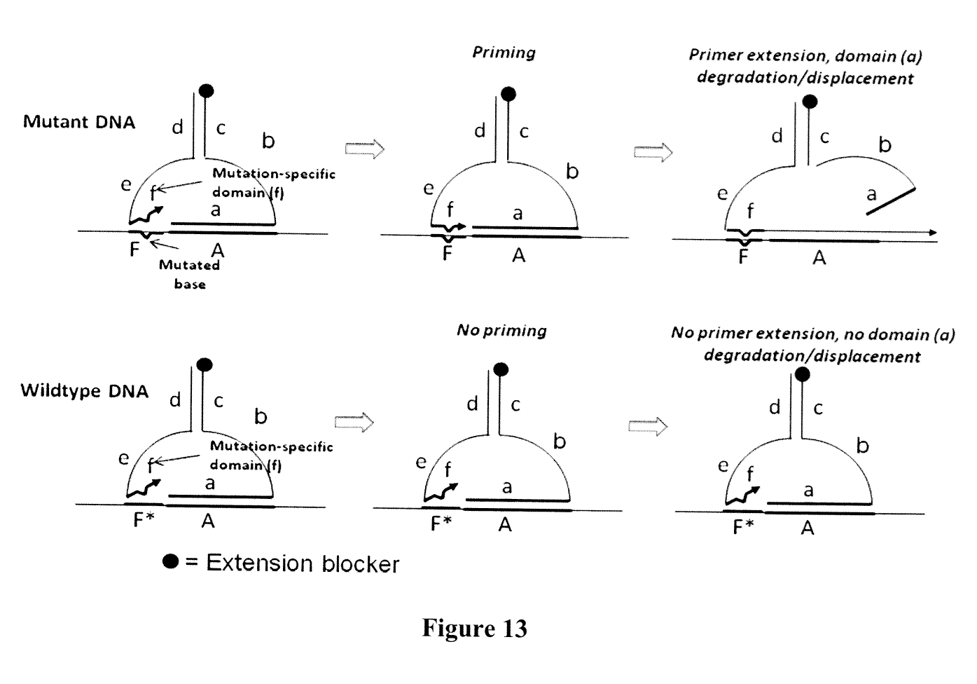

In a further embodiment, a polynucleotide primer combination of the disclosure is provided in which region (A) is 5′ to region (F) in the target polynucleotide. This polynucleotide primer combination is, in some aspects, contemplated for use in detecting a mutation in a target polynucleotide versus a non-target polynucleotide (FIG. 13). Due to the flexibility of domain (e), the interaction of short domain (f) with target polynucleotide region (F) is highly sensitive to any defect within region (F). In the absence of mismatches within the short duplex between domain (f) and region (F), the formed priming complex (see FIG. 13) initiates extension of a product polynucleotide from the 3′ end of domain (f). If region (F) has a mutation, and corresponding mutation-specific domain (f) of the second polynucleotide (2) forms a stable duplex with mutated region (F), the formed primer complex can initiate extension of a product polynucleotide and degradation and/or displacement of domain (a) from the 3′ end of the mutation-specific domain (f). If region (F) has no mutation (i.e., is a wildtype sequence (F*)), the mutation-specific domain (f) of the second polynucleotide (2) cannot form a stable duplex with region (F*) and cannot initiate extension of a product polynucleotide and degradation and/or displacement of domain (a) from the 3′ end of the mutation-specific domain (f) (FIG. 13).

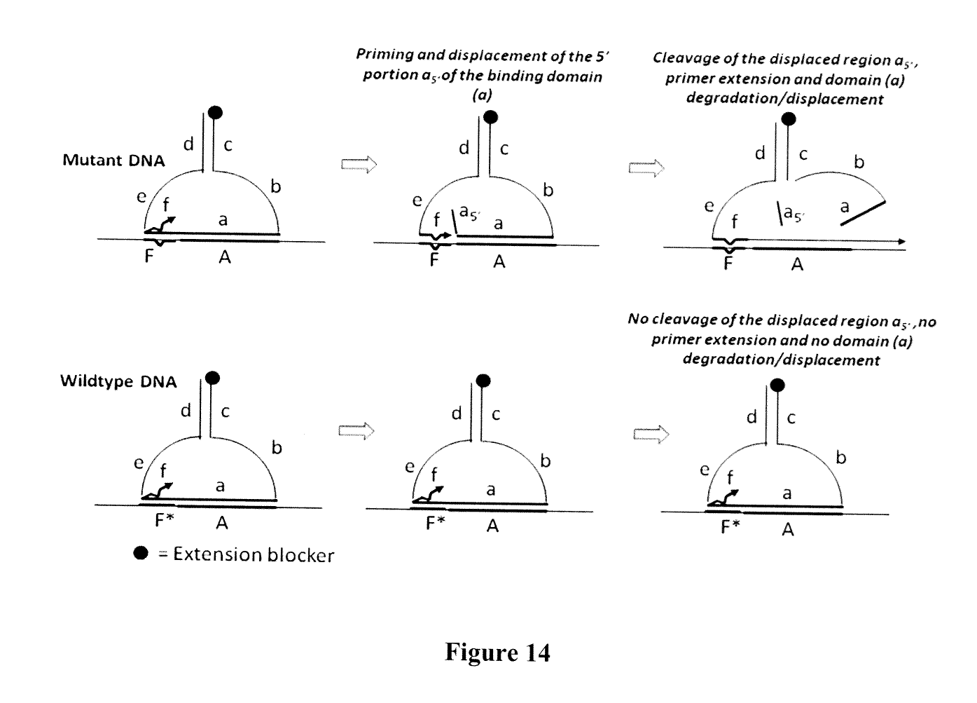

In another embodiment, a polynucleotide primer combination of the disclosure is provided in which region (A) is 5′ to region (F) in a target polynucleotide, it is further contemplated that at least a portion of domain (a) acts as a blocker polynucleotide (FIG. 14). Use of a blocker polynucleotide enables the detection of a few or even single mutant target polynucleotides in the presence of a very large number of non-target polynucleotides, for example and without limitation 1 target polynucleotide: 10⁴ non-target polynucleotides, or 1 target polynucleotide: 10⁵ non-target polynucleotides. Ratios of target to non-target polynucleotides that can be detected by polynucleotide primer combinations of the disclosure are from at least about 1:10 to at least about 1:10¹⁰.

In some embodiments, the length of domain (a) of the first polynucleotide (1) is extended in the 5′ direction, allowing domain (a) of the first polynucleotide (1) to overlap with domain (f) of the second polynucleotide (2). In these aspects, domain (a) serves as a blocker oligonucleotide that increases the specificity of mutation selection during primer extension and PCR.

The blocking portion (a_{5′}) of domain (a) is in various aspects designed to be complementary to a region (F*) in a non-target polynucleotide and include a mismatch with a region (F) in a target polynucleotide. The blocking portion (a_{5′}) of domain (a) interacts with region (F*) or a part of region (F*) that comprises a variant base, thus overlapping completely or partially with domain (f) of the second polynucleotide (2). Blocking portion (a_{5′}) of domain (a), in various aspects, comprises a modified base, including but not limited to LNAs and PNAs.

As shown in FIG. 14, with respect to a target polynucleotide, mutation-specific domain (f) first invades region (F) and displaces the imperfectly annealed 5′ tail of domain (a) of the first polynucleotide (1). Then a DNA polymerase with 5′ to 3′ exonuclease activity (for example and without limitation, Tag DNA polymerase) cleaves the displaced region (a_{5′}) of domain (a) of the first polynucleotide (1). Next, the polymerase replicates the polynucleotide template in a nick-translation mode until domain (a) of the first polynucleotide (1) becomes too short to maintain a stable interaction with the target polynucleotide and dissociates from the template allowing the polymerase to proceed in replication mode. In the case of a non-target polynucleotide, domain (f) is unable to invade and displace the perfectly annealed domain (a) of the first polynucleotide (1), and thus cannot prime polynucleotide extension. Accordingly, use of domain (a) with a blocking function results in improved discrimination of a rare target polynucleotide in the presence of abundant non-target polynucleotides using a polynucleotide primer combination as described above.

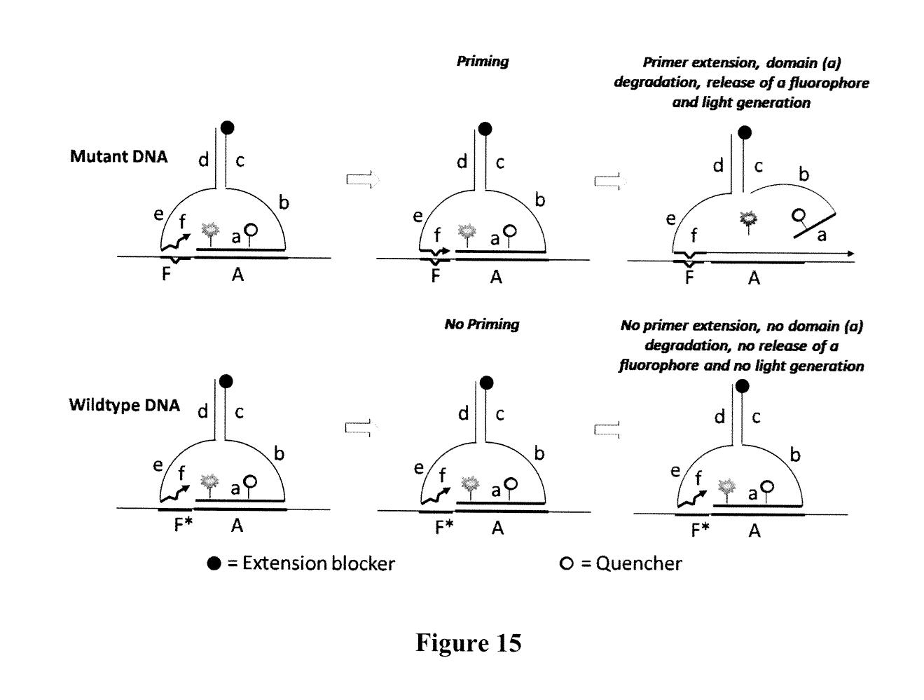

In further embodiments, a polynucleotide primer combination is provided wherein domain (a) further comprises a detectable marker and a quencher that quenches the detectable marker. As shown in FIG. 15, incorporation of a detectable marker and quencher into binding domain (a) adds a domain (a) 5′-detection probe function to a polynucleotide primer combination. Detectable marker and quencher molecules can be positioned as shown in FIG. 15, or in some aspects can be positioned in the reverse orientation (i.e., detectable marker can be positioned 3′ of the quencher). A short distance (for example and without limitation, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 or more nucleotides) between the detectable marker and quencher within domain (a) would result in quenching of the detectable marker and absence of detection. Upon primer extension and degradation of domain (a) the detectable marker or the quencher will be released into solution and generate detectable signal. Extension of domain (f) would result in a detectable signal generated from the target polynucleotide. Lack of extension of domain (f) results in no detectable signal from the non-target polynucleotide. For generation of a detectable signal by primer combinations containing detectable marker and quencher molecules within domain (a), it is important for the polymerase used for extension or amplification to have intrinsic 5′ to 3′ exonuclease activity. Examples of thermophilic DNA polymerases with 5′ to 3′ exonuclease activity include but are not limited to Taq DNA polymerase, Tth DNA polymerase, family of DyNAzyme DNA polymerases from Termus brockianus, Bst DNA polymerase, holoenzyme; a non-limiting example of a mesophilic DNA polymerase with 5′ to 3′ exonuclease activity is E. coli DNA polymerase I.

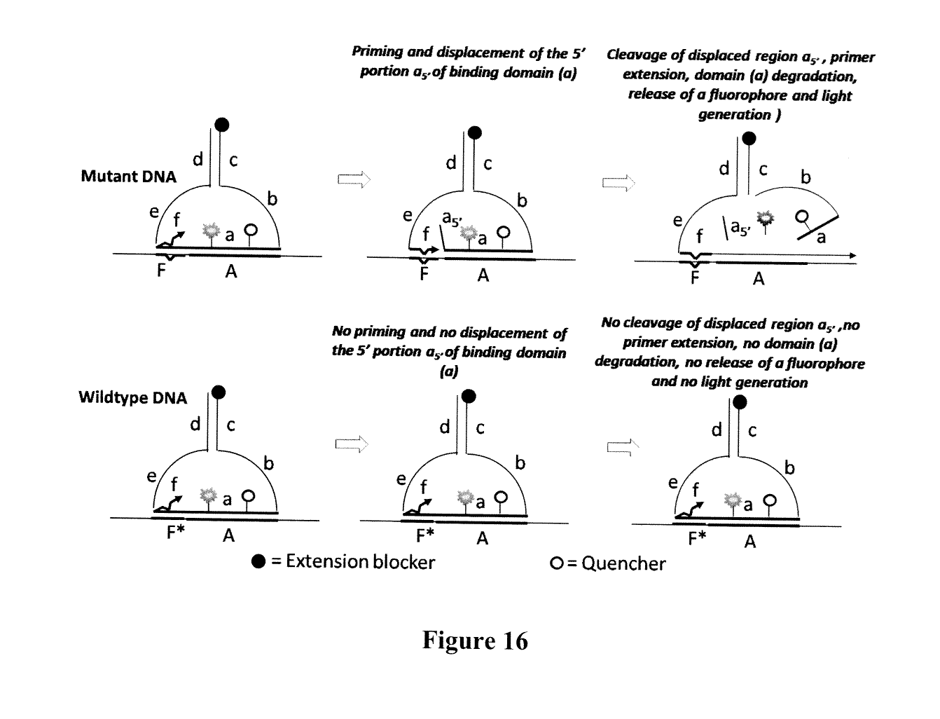

In some embodiments, a polynucleotide primer combination is provided wherein domain (a) further comprises a detectable marker and a quencher that quenches the detectable marker, and wherein domain (a) further comprises a blocker polynucleotide domain (a_{5′}) as described above, and depicted in FIG. 16.

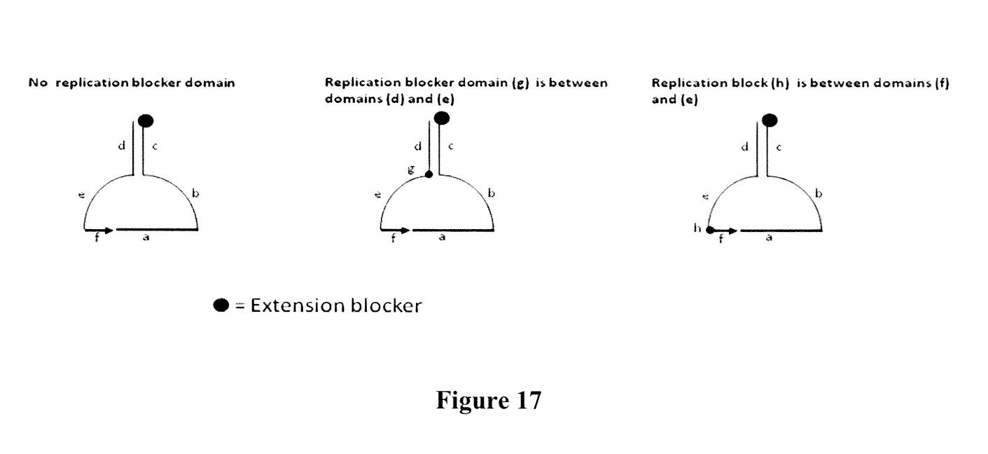

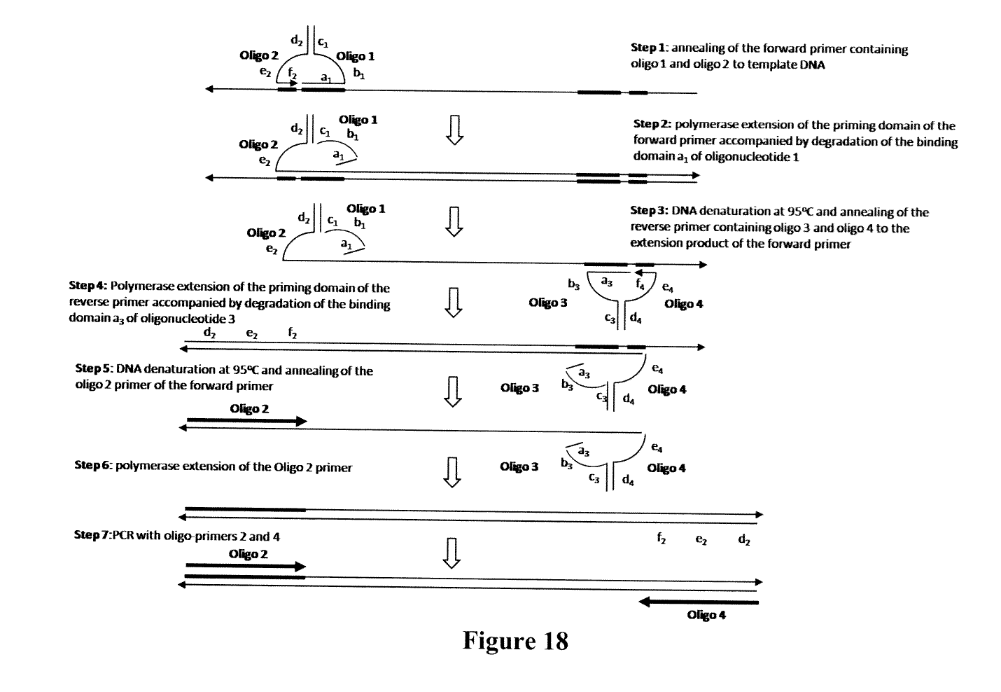

A polynucleotide primer combination is also provided, in certain embodiments, that further comprises a replication blocker domain within the second polynucleotide (2) (see FIG. 17). A polynucleotide primer combination can thus have an additional element, specifically a replication blocker domain, located either between domains (d) and (e) (i.e., domain (g)), or between domains (f) and (e) (i.e., domain (h)). The replication blocker domain is, in various aspects, an abasic site, a modified base or linker, or a polymer that does not allow DNA polymerase to replicate beyond but rather terminates replication at the position of the (g) or (h) domain. The purpose of replication blocker domain is to prevent DNA polymerase from replicating domain (d) (in the case of domain (g)), or from replicating domains (e) and (d) (in the case of domain (h)). Use of a polynucleotide primer combination without a replication blocker domain is depicted in FIG. 18. Polynucleotide primer combinations of the disclosure can be used for quantitative PCR, mutation-specific PCR, mutation-specific PCR enhanced by an overlapping blocking domain, and for mutation-specific PCR with combined blocking domain and detection probe function. During the first 6 steps of the PCR process described in FIG. 18, two polynucleotide primer combinations (forward and reverse) facilitate formation of the PCR amplicon. After formation of the PCR amplicon, further amplification can be supported by polynucleotide primer combinations 2 and 4 without absolute necessity of first and third polynucleotides (1) and (3) (although in some aspects they can still participate as fluorescent probes or blockers, and help to generate a specific signal).

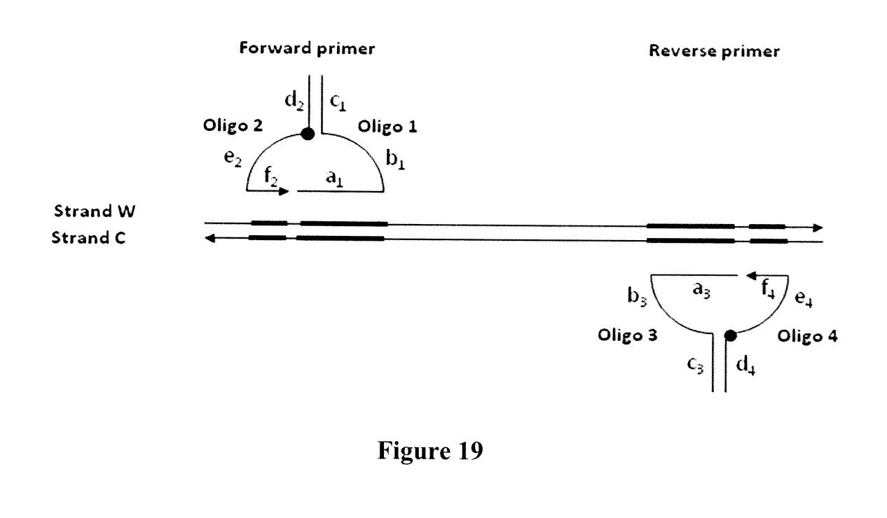

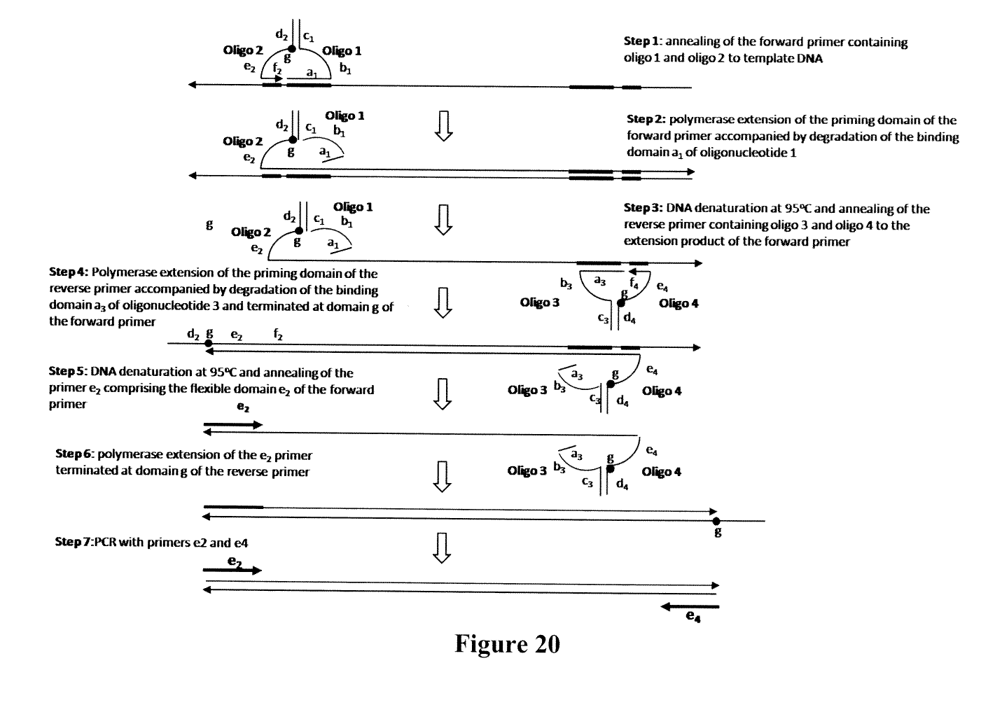

In embodiments wherein the second polynucleotide (2) comprises a replication blocker domain (g), FIGS. 19 and 20 depict their use in PCR. A polynucleotide primer combination with a (g) domain replication blocker can be used for quantitative PCR, mutation-specific PCR, mutation-specific PCR enhanced by an overlapping blocking domain, and for mutation-specific PCR with the combined blocking and detection probe function. During the first 6 steps of the PCR process described in FIG. 20, two primers (forward and reverse) facilitate formation of the PCR amplicon. After formation of the PCR amplicon, further amplification can be supported by universal primers e₂ and e₄.

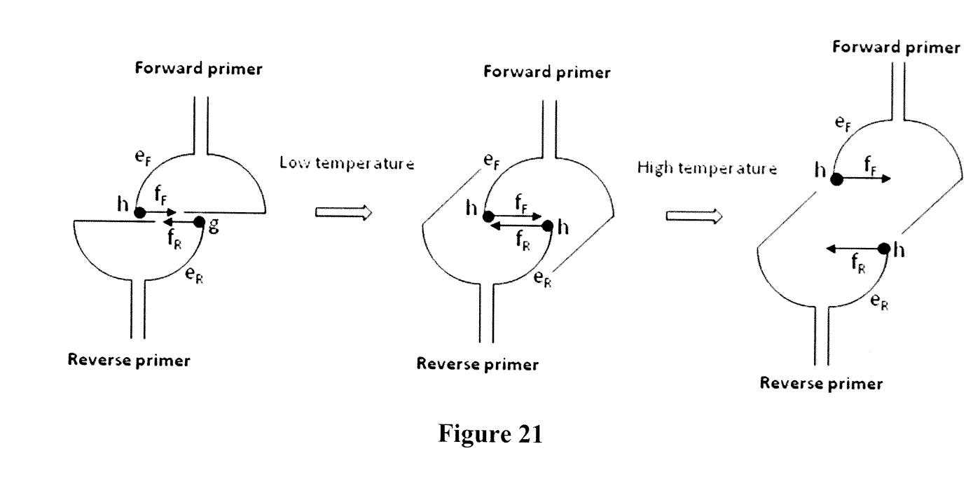

The disclosure also provides a polynucleotide primer combination wherein the second polynucleotide (2) further comprises a replication blocker domain (h), wherein domain (h) is positioned between domains (e) and (f) (FIG. 21). The potential for a polynucleotide primer combination of the disclosure to form a primer-dimer at low temperature cannot be amplified at a higher temperature due to the presence of the replication blocker domain (h).

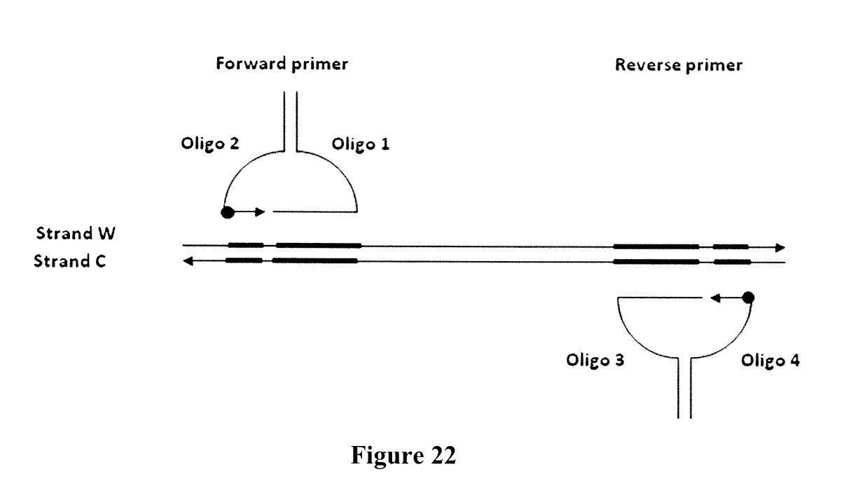

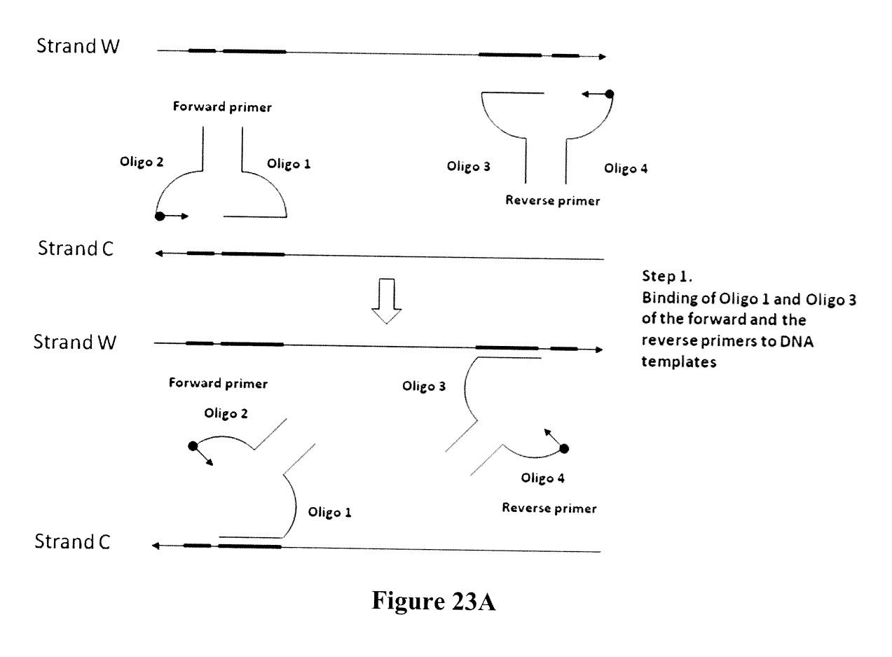

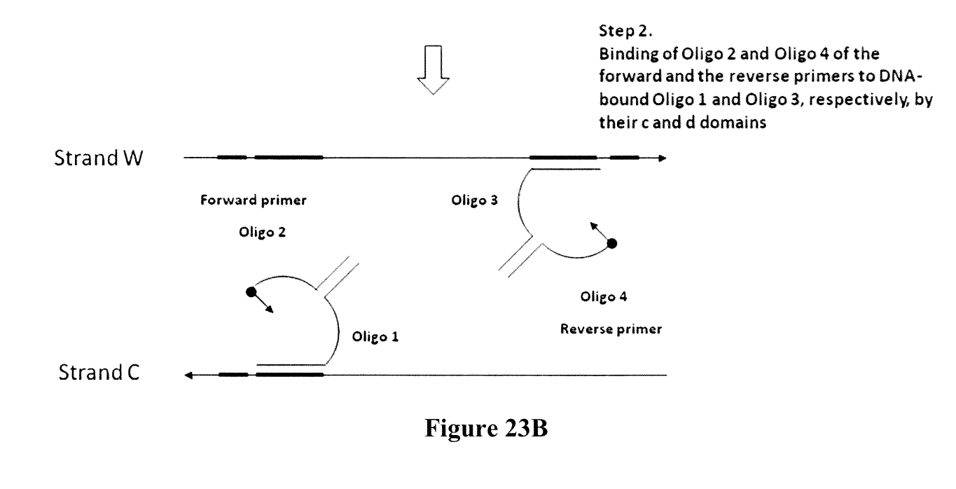

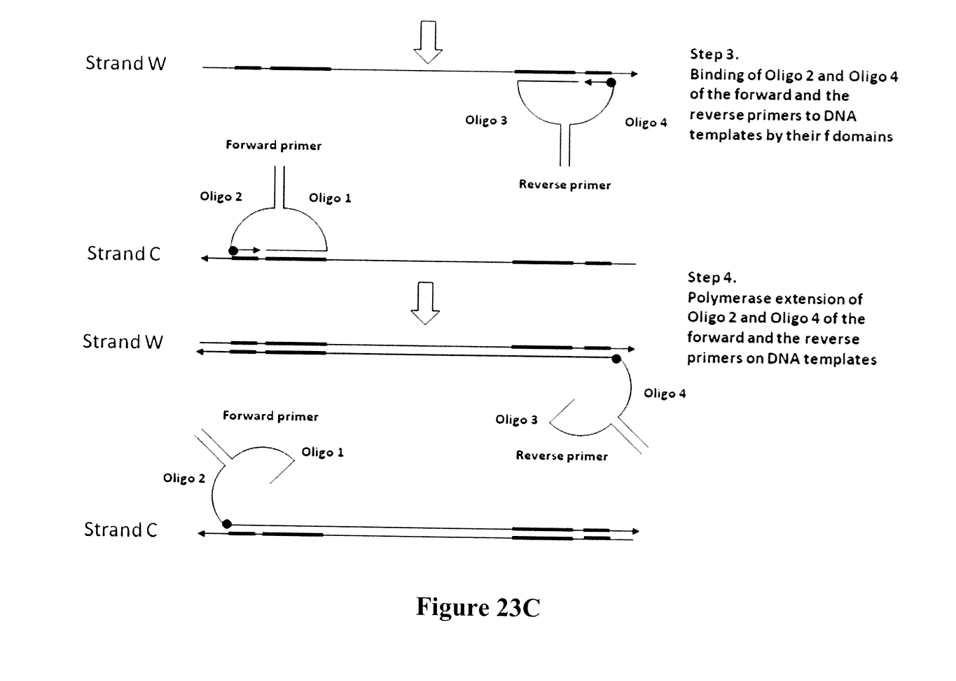

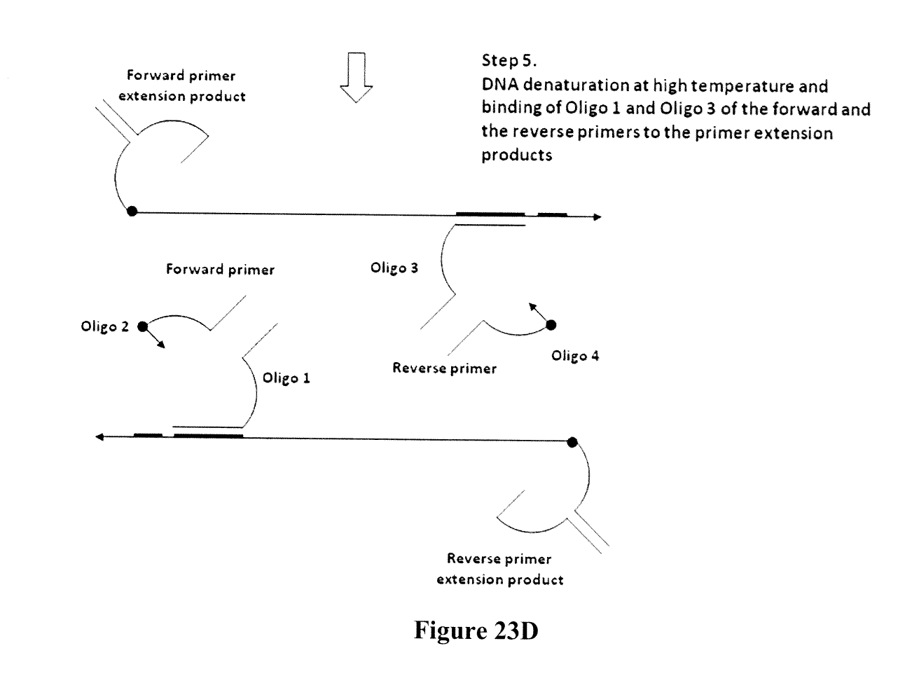

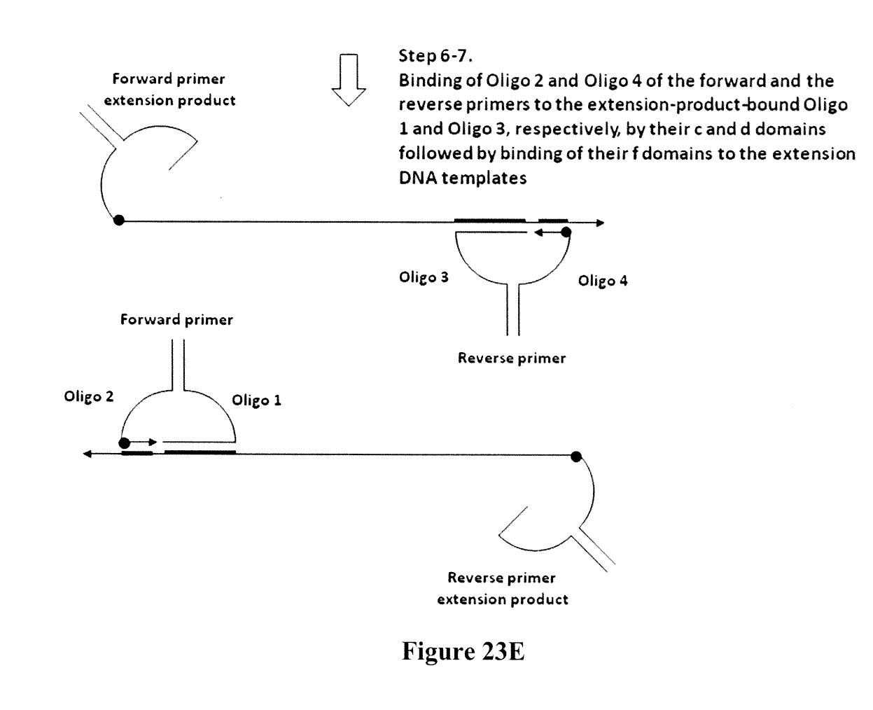

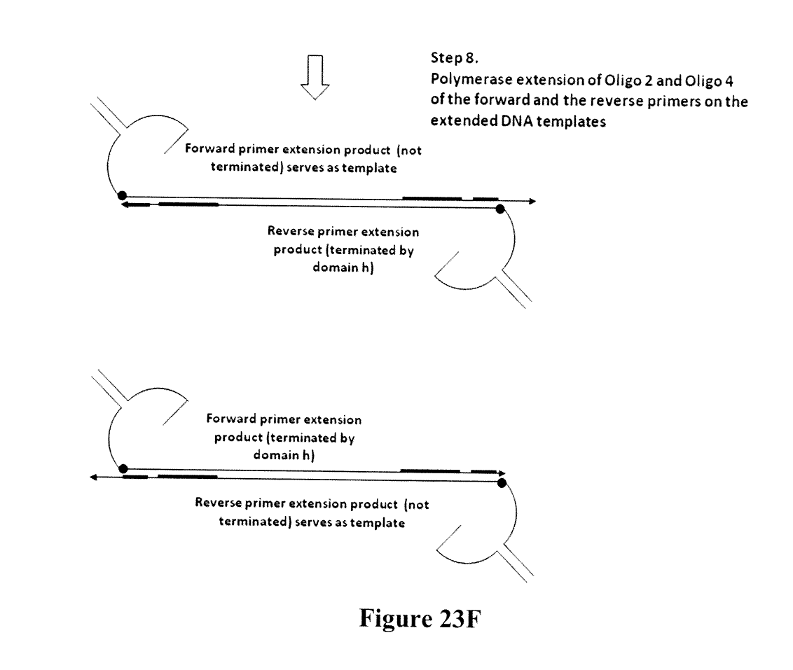

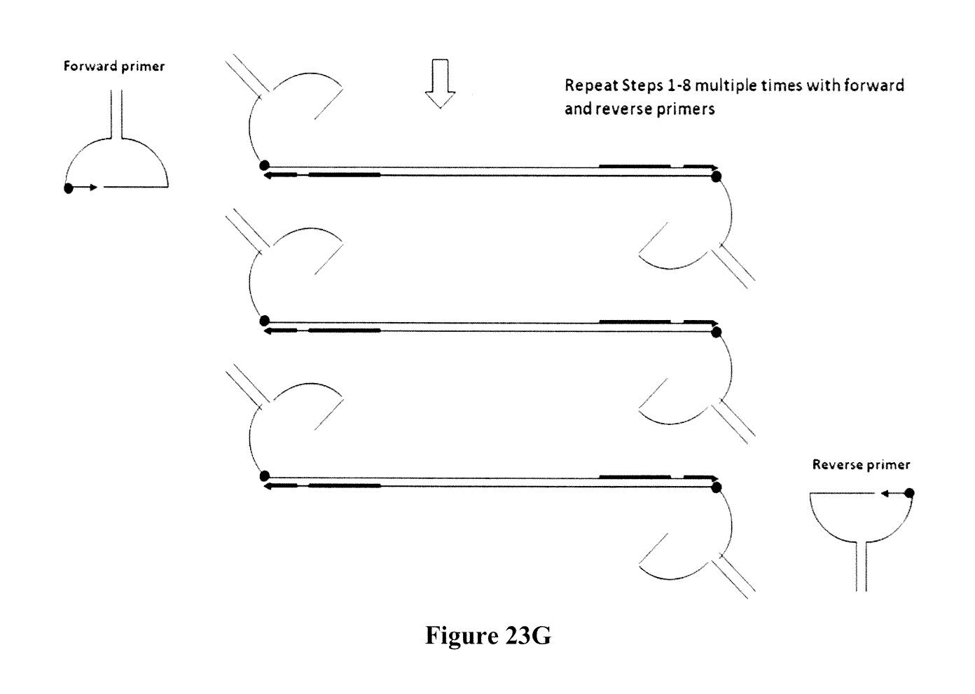

In embodiments wherein the second polynucleotide (2) comprises a replication blocker domain (h), FIGS. 22 and 23 A-G depict their use in PCR. In the case of polynucleotide primer combinations without a replication blocking domain or with a (g) domain, fully-assembled polynucleotide primer combinations are only necessary for the first few cycles of the PCR process. Later PCR amplification can be supported by the second polynucleotide (2) alone or by primers corresponding to their internal (e) domain sequences. Polynucleotide primer combinations with an (h) domain function differently. They cannot support PCR without the presence of the first and third polynucleotides (1) and (3) during the whole process due to the very short size of their functional priming domain. For this reason, any primer-dimers formed by a polynucleotide primer combination that contains an (h) domain cannot be amplified at temperatures typical for a standard PCR reaction (>50° C.). For example and without limitation, a polynucleotide primer combination with an (h) domain can be used for quantitative PCR, mutation-specific PCR, mutation-specific PCR enhanced by an overlapping blocking domain, and for mutation-specific PCR with combined blocking and detection probe domains.

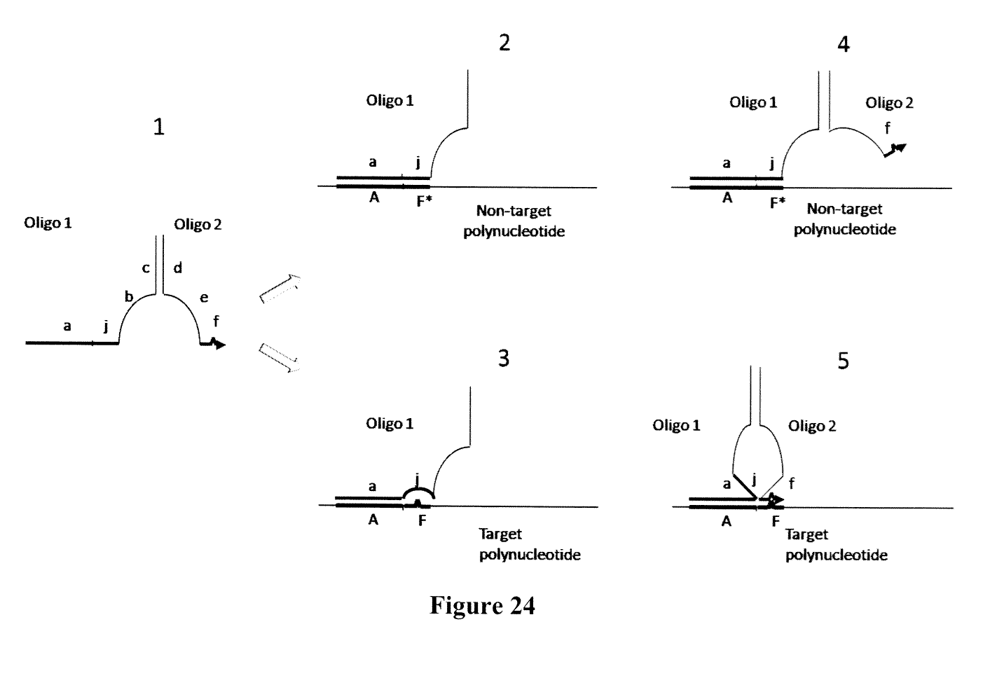

In some embodiments, a polynucleotide primer combination is provided wherein the first polynucleotide (1) comprises a self-blocking domain (FIG. 24). A polynucleotide primer combination with a self-blocking domain represents a case where the first polynucleotide (1) plays a dual function: as a stable binding domain (a) and as a competitive blocker domain for the target polynucleotide specific domain (f) of the second polynucleotide (2). To achieve this goal the polynucleotide primer combination is designed such that the binding domain (a) of the first polynucleotide (1) has two adjacent domains: binding domain (a) and binding-blocking domain (j) that are fully complementary to two adjacent target regions (A) and (F*) of the non-target polynucleotide, and not fully complementary to the (F) region of the target polynucleotide. As a result, the first polynucleotide (1) will form a stable duplex between domains (a) and (j) and (A) and (F*) of the non-target polynucleotide and a destabilized duplex between domain (f) and region (F) of the target polynucleotide. Formation of a stable complex between the first polynucleotide (1) and the non-target polynucleotide within regions (A) and (F*) will outcompete formation of a less stable duplex between the priming domain (f) of the second polynucleotide (2) and the non-target polynucleotide, thus preventing extension of domain (f) on the non-target polynucleotide. Formation of an unstable duplex between domain (j) of the first polynucleotide (1) and the mismatched region (F) of the target polynucleotide will promote competition between domain (f) of the second polynucleotide (2) and domain (j) of the first polynucleotide (1). This results in formation of a stable (f)/(F) duplex and extension of domain (f) on the target polynucleotide. As a result, self-blocking polynucleotide primer combinations provide greater specificity for amplification and detection of target alleles that are present at very low copy number in the presence of a large number of non-target alleles.

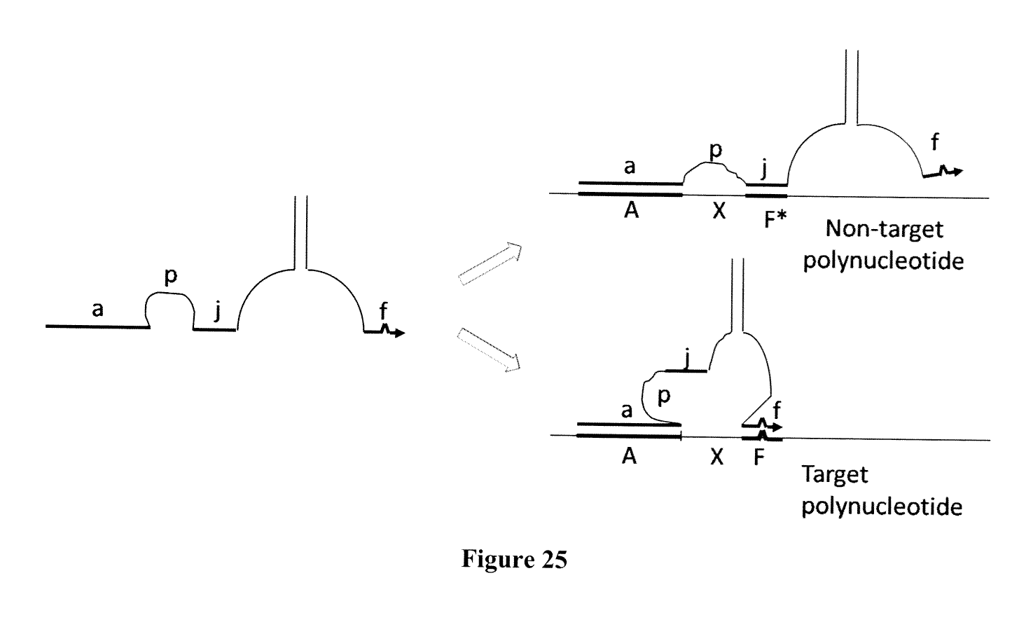

In a related embodiment, a polynucleotide primer combination is provided wherein the first polynucleotide (1) comprises non-contiguous domains (a) and (j) (FIG. 25). The situation presented in FIG. 25 differs from that described in FIG. 24 only by the fact that the two target regions (A) and (F) are not adjacent to one another but are separated by region (X). To adjust to this difference, the self-blocking first polynucleotide (1) depicted in FIG. 25 has a first polynucleotide (1) portion with three domains: domains (a) and (j) that are fully complementary to regions (A) and (F*) of the non-target polynucleotide, and domain (p) that represents a unique non-complementary spacer between domains (a) and (j). The self-blocking polynucleotide primer combination described in FIG. 25 functions similarly to the primer combination described in FIG. 24.

### B. Simple Second Strand Synthesis

In another embodiment, a method of amplifying a target polynucleotide is provided using the first (1) and second polynucleotides (2) comprising contacting the target polynucleotide with the first (1) and second polynucleotides (2) disclosed herein under conditions sufficient to allow hybridization of the first domain (a) of the first polynucleotide (1) to the first target polynucleotide region (A) of the target polynucleotide and the first domain (f) of the second polynucleotide (2) to the second target polynucleotide region (F) of the target polynucleotide, and extending the first domain (f) (i.e., priming domain) of the second polynucleotide with a DNA polymerase under conditions which permit extension of the first domain of the first polynucleotide. In some aspects, the second polynucleotide (2) (with associated polynucleotide product extended therefrom) and first polynucleotide are then denatured from the target polynucleotide and another set of first and second polynucleotides are allowed to hybridize to a target polynucleotide.

In one aspect, the first polynucleotide (1) and the second polynucleotide (2) hybridize sequentially to the target polynucleotide. In another aspect, the first domain (a) of the first polynucleotide (1) hybridizes to the target before the first domain (f) of the second polynucleotide (2) hybridizes to the target polynucleotide. In yet another aspect, the first domain (f) of the second polynucleotide (2) hybridizes to the target polynucleotide before the first domain (a) of the first polynucleotide (1) hybridizes to the target polynucleotide. In another aspect, the first domain (a) of the first polynucleotide (1) and the first domain (f) of the second polynucleotide (2) hybridize to the target polynucleotide concurrently.

In various embodiments, the target polynucleotide includes but is not limited to chromosomal DNA, genomic DNA, plasmid DNA, cDNA, RNA, a synthetic polynucleotide, a single stranded polynucleotide, or a double stranded polynucleotide. In one aspect, the target is a double stranded polynucleotide and the first domain (a) of the first polynucleotide (1) and the first domain (f) of the second polynucleotide (2) hybridize to the same strand of the double stranded target polynucleotide. In another aspect, the second domain (c) of the first polynucleotide (1) and the second domain (d) of the second polynucleotide (2) hybridize prior to hybridization of the first polynucleotide (1) and the second polynucleotide (2) to the target polynucleotide.

In an embodiment, the first polynucleotide (1) and the second polynucleotide (2) hybridize to the target polynucleotide concurrently and the third polynucleotide (3) and the fourth polynucleotide (4) hybridize to the complement of the target polynucleotide concurrently, the first polynucleotide (1) and the second polynucleotide (2) hybridizing to the target polynucleotide at the same time that the third polynucleotide (3) and the fourth polynucleotide (4) hybridize to the complement of the target polynucleotide.

In another embodiment, the first polynucleotide (1), the second polynucleotide (2), the third polynucleotide (3) and the fourth polynucleotide (4) do not hybridize to the target polynucleotide and the complement of the target polynucleotide at the same time.

In yet another embodiment, the second domain (c) of the first polynucleotide (1) and the second domain (d) of the second polynucleotide (d) hybridize prior to hybridizing to the target polynucleotide. In another embodiment, the second domain (c) of the third polynucleotide (3) and the second domain (d) of the fourth polynucleotide (4) hybridize prior to hybridizing to the complement of the target polynucleotide.

In an embodiment, the second domain (c) of the first polynucleotide (1) and the second domain (d) of the second polynucleotide (2) hybridize prior to hybridizing to the target polynucleotide and the second domain (c) of the third polynucleotide (3) and the second domain (d) of the fourth polynucleotide (4) hybridize prior to hybridizing to the complement of the target polynucleotide.

In another embodiment, the target polynucleotide contains a mutation in the region to which the first domain (f) of the second polynucleotide hybridizes to the target polynucleotide. In some embodiments, the target polynucleotide is fully complementary in the region to which the first domain (f) of the second polynucleotide (2) hybridizes to the target polynucleotide. In some embodiments, the non-target polynucleotide is not fully complementary in the region to which the first domain (f) of the second polynucleotide (2) hybridizes to the non-target polynucleotide. In another embodiment, the target polynucleotide contains a mutation in the region to which the first domain (f) of the fourth polynucleotide (4) hybridizes to the target polynucleotide. In some aspects, the mutation is a destabilizing mutation, an insertion, a deletion, a substitution or an inversion. In related aspects, the destabilizing mutation prevents extension of the second polynucleotide (2), or the fourth polynucleotide (4), or both.

### C. Multiplexing

In an embodiment, the extension by an enzyme that is capable of synthesizing a nucleic acid is a multiplex extension, the first domain (f) of the second polynucleotide having the property of hybridizing to more than one region in the target polynucleotide. In a related embodiment, the extension by an enzyme that is capable of synthesizing a nucleic acid is a multiplex extension, the first domain (f) of the fourth polynucleotide (4) having the property of hybridizing to more than one locus in the target polynucleotide.

In related embodiments, multiplex PCR is performed using at least two polynucleotide primers to amplify more than one polynucleotide product. In some aspects of these embodiments, each polynucleotide primer used for multiplex PCR is a polynucleotide combination as disclosed herein. In other aspects, at least one polynucleotide primer used for multiplex PCR is a polynucleotide combination as disclosed herein.

In another embodiment, multiplex PCR is performed using multiple fixer polynucleotides and are directed against genomic repeated sequences. In another embodiment, the fixer polynucleotides are comprised of random sequences. In some of these aspects, multiple fixer polynucleotides refers to about 10 polynucleotide sequences. In other aspects, multiple fixer polynucleotides refers to about 15, about 20, about 25, about 30, about 35, about 40, about 45, about 50, about 55, about 60, about 65, about 70, about 75, about 80, about 85, about 90, about 95, about 100, about 150, about 200, about 250, about 300, about 350, about 400, about 450, about 500, about 550, about 600, about 650, about 700, about 750, about 800, about 850, about 900, about 950, about 1000 or more polynucleotide sequences. These fixer polynucleotide sequences would provide a multitude of “fixed” locations in the genome to which a multitude of primer polynucleotides could then bind, taking advantage of the unique complementary polynucleotide sequences present in both the primer and fixer polynucleotides as described herein.

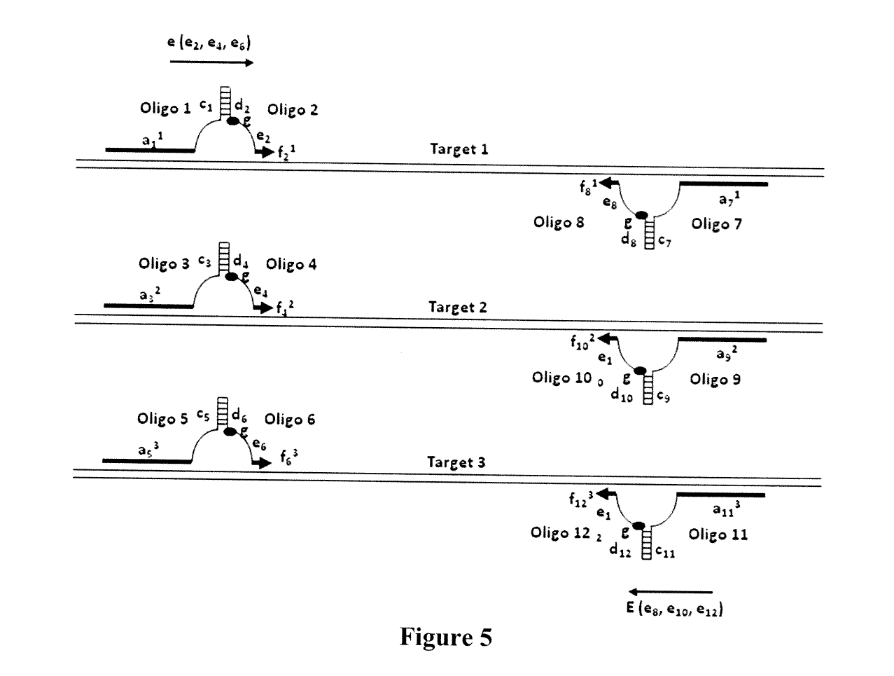

Thus, in an embodiment, multiple target polynucleotide sequences can be analyzed in a single reaction vessel. In some aspects, this multiplex reaction is performed with a polynucleotide primer combination of the disclosure that further comprises a replication blocker. In one aspect, the replication blocker is domain (g) as described herein. FIG. 5 depicts three forward polynucleotide primer combinations of the disclosure for the multiplexed PCR that have flexible domains (e₂), (e₄) and (e₆), each with the same universal sequence ‘e’. Three reverse polynucleotide primer combinations of the disclosure that are used for the multiplexed PCR are also depicted in FIG. 5, which have flexible domains (e₈), (e₁₀) and (e₁₂) each with the same universal sequence ‘E’ that is different from universal sequence ‘e’. After the first three PCR cycles, all three targeted amplicons will be terminated by two universal sequences ‘e’ and ‘E’, so further amplification of all amplicons can be supported by the two universal primers e and E. These aspects involve three co-amplified targeted regions, but a worker of ordinary skill in the art will appreciate that this number can be substantially greater and involve 10, 100, 1,000, 10,000 or more targeted regions in one multiplexed PCR.

### D. Real-Time PCR

Primer combinations of the disclosure are useful for real-time PCR. Analysis and quantification of rare transcripts, detection of pathogens, diagnostics of rare cancer cells with mutations, or low levels of aberrant gene methylation in cancer patients are among the problems that can be solved by improved real-time PCR assays that combine high sensitivity and specificity of target amplification, high specificity of target detection, the ability to selectively amplify and detect a small number of cancer-specific mutant alleles or abnormally methylated promoters in the presence of thousands of copies of normal DNA, analysis and quantification of low copy number RNA transcripts, detection of fluorescence traces the ability to multiplex 4-5 different targets in one assay to maximally utilize capabilities of current real-time thermal cyclers. A fluorophore is positioned at the 5′ end of the second polynucleotide (2), and a quencher is positioned at the 3′ end of the first polynucleotide (1). In this arrangement, no fluorescence is detected when the first (1) and second (2) polynucleotides are hybridized (since the fluorophore is positioned adjacent to the quencher). However, following extension of the primer polynucleotide during PCR, the first polynucleotide (1) and second polynucleotide (2) will become separated during the denaturation phase of PCR, thus creating distance between the fluorophore and the quencher and resulting in a detectable fluorescent signal.

Primer combinations of the disclosure and a probe polynucleotide can also be used for real-time PCR. The probe polynucleotide is labeled with a fluorophore on its 5′ end, a quencher on its 3′ end, and in some embodiments, an additional internal quencher. When the first polynucleotide is extended by a polymerase with 5′ to 3′ exonuclease activity, such as Tag polymerase, the label is cleaved and is no longer quenched, resulting in increased signal from the label. In some embodiments, the probe polynucleotide is a molecular beacon probe. In short, a molecular beacon probe is comprised of a nucleotide sequence with bases on its 5′ and 3′ ends that are complementary and form a hairpin structure in the absence of a target polynucleotide. The molecular beacon probe also comprises a quencher at its 3′ end (or 5′ end) and a fluorescent label at its 5′ end (or 3′ end) such that there is no detectable signal from the label when the target polynucleotide is not present. The molecular beacon probe also comprises a sequence that is complementary to the target polynucleotide such that, in the presence of the target, hybridization of the probe to the target polynucleotide causes the dissociation of the hairpin structure and loss of quenching, resulting in a detectable fluorescent signal.

Primer combinations of the disclosure and a blocker polynucleotide can also be used in combination for real-time PCR. The primer polynucleotide (i.e., “second polynucleotide (2)”) is labeled with a fluorophore on its 5′ end, and the fixer polynucleotide (i.e., “first polynucleotide (1)”) is labeled with a quencher on its 3′ end. The blocker polynucleotide is complementary to a target polynucleotide region located immediately 5′ of the second target polynucleotide region (F). In some embodiments, the blocker polynucleotide overlaps with the first domain (f) of the second polynucleotide (2). In other words, the nucleotide(s) at the 3′ end of the second polynucleotide (2) and the nucleotide(s) at the 5′ end of the blocker polynucleotide would be complementary to the same nucleotide(s) of the target polynucleotide. In related embodiments, the nucleotide(s) at the 3′ end of the second polynucleotide (2) and the nucleotide(s) at the 5′ end of the blocker polynucleotide are different. In these embodiments, the nucleotide(s) at the 3′ end of the second polynucleotide (2) would hybridize to the target polynucleotide when it is complementary to the target polynucleotide at the appropriate position(s), thus allowing for extension of the second polynucleotide under the appropriate conditions. Following extension of the primer polynucleotide during PCR, the primer polynucleotide and fixer polynucleotide will become separated during the denaturation phase of PCR, thus creating distance between the fluorophore and the quencher and resulting in a detectable fluorescent signal. In related embodiments, the nucleotide at the 5′ end of the blocker polynucleotide would hybridize to the non-target polynucleotide when it is complementary to the non-target polynucleotide at the appropriate position, thus blocking extension of the second polynucleotide. In this arrangement, no fluorescence is detected when the primer and fixer polynucleotides are hybridized (since the fluorophore is positioned adjacent to the quencher). In various embodiments, the nucleotide at the 3′ end of the blocker polynucleotide is modified to prevent extension by a polymerase. This system allows for detection of, for example and without limitation, single nucleotide polymorphisms with great sensitivity and specificity.

Primer combinations of the disclosure, blocker polynucleotides, and probe polynucleotides are also used in combination for real-time PCR. In related embodiments, the second polynucleotide (2) used in this combination comprises a modified nucleotide as the nucleotide at its 3′ end and the blocker polynucleotide comprises a modified nucleotide as the nucleotide at its 5′ end. In various aspects, the second polynucleotide (2) and/or the blocker polynucleotide comprises at least one modified nucleotide that is 1, 2, 3, 4, or 5 nucleotides from their 3′ or 5′ end, respectively. In some embodiments, the modified nucleotide is a locked nucleic acid.

In some aspects, the above embodiments further comprise a reverse primer polynucleotide. The reverse primer is complementary to a region in the polynucleotide created by extension of the second polynucleotide. As is apparent, in some embodiments the reverse primer is also complementary to the complementary strand of the target polynucleotide when the target polynucleotide is one strand of a double-stranded polynucleotide. Inclusion of a reverse primer allows for amplification of the target polynucleotide. In various aspects, the reverse primer is a “simple” primer wherein the sequence of the reverse primer is designed to be sufficiently complementary over its entire length to hybridize to a target sequence over the entire length of the primer. A simple primer of this type is in one aspect, 100% complementary to a target sequence, however, it will be appreciated that a simple primer with complementarity of less than 100% is useful under certain circumstances and conditions.

In other aspects, a reverse primer is a separate polynucleotide primer combination that specifically binds to regions in a sequence produced by extension of a polynucleotide from the first domain (f) of the second polynucleotide (2) in a primer pair combination used in a first reaction.

In various aspects, the methods described herein provide a change in sequence detection from a sample with a non-target polynucleotide compared to sequence detection from a sample with a target polynucleotide. In some aspects, the change is an increase in detection of a target polynucleotide in a sample compared to sequence detection from a sample with a non-target polynucleotide. In some aspects, the change is a decrease in detection of a target polynucleotide in a sample compared to sequence detection from a sample with a non-target polynucleotide.

Due to the increased specificity of the polynucleotides described herein, real-time PCR can be performed in the presence of SYBR green dye to achieve a specificity that is equivalent to that achieved using TaqMan, molecular beacon probes or Scorpion primers but at a greatly reduced cost.

In one embodiment, the primer polynucleotide (i.e., “second polynucleotide (2)”) is labeled with a fluorescent molecule at its 5′ end and a second quenching polynucleotide (i.e., “universal quencher polynucleotide”) that is labeled at its 3′ end with a quencher are both hybridized to the second domain (c) of the fixer polynucleotide (i.e., “first polynucleotide (1)”), which comprises a blocking group at its 3′ end to prevent extension from a DNA polymerase. This complex has no fluorescence in this state but will fluoresce when the complex is displaced (denatured) following extension of the primer polynucleotide by a DNA polymerase.

In another embodiment, the primer polynucleotide (i.e., “second polynucleotide (2)”) comprising a fluorophore at its 5′ end is hybridized to a fixer polynucleotide (i.e., “first polynucleotide (1)”) comprising a quencher at its 3′ end. The complex has no fluorescence when hybridized, but will fluoresce when the complex is displaced (denatured) following extension of the primer polynucleotide by a DNA polymerase. In another aspect of the method, multiplex real-time PCR is performed using two sets of polynucleotide combinations, wherein one polynucleotide in each primer set is labeled with a fluorophore, and the two fluorophores are distinguishable from each other.

In another embodiment, the primer polynucleotide (i.e., “second polynucleotide (2)”) comprises a fluorophore, a quencher on its 3′ end, and these two labels are separated by a stretch of RNA or RNA/DNA oligonucleotides (i.e., “probe polynucleotide”). In some aspects, the probe polynucleotide further comprises an internal Zen quencher. In some aspects, a fluorescent signal is generated upon creation and degradation of the RNA/DNA hybrid by a thermostable RNase H and release of a free fluorophore (or quencher) into solution.

In some embodiments, one fixer polynucleotide (i.e., “first polynucleotide (1)”) may be used in combination with 2, 3, 4, 5 or more primer polynucleotides (i.e., “second polynucleotides (2)”) for simultaneous multiplex detection of several mutations in one real-time PCR assay.

In another embodiment, a kit is provided comprising, e.g., a package insert and any of the primer combinations of the disclosure, which can in various aspects be fluorescently labeled).

### E. Primer Extension

The primer compositions disclosed herein can be used in any method requiring or utilizing primer extension. For example, primer extension can be used to determine the start site of RNA transcription for a known gene. This technique requires a labeled primer polynucleotide combination as described herein (usually 20-50 nucleotides in length) which is complementary to a region near the 3′ end of the gene. The polynucleotide combination is allowed to anneal to the RNA and reverse transcriptase is used to synthesize complementary DNA (cDNA) to the RNA until it reaches the 5′ end of the RNA. By analyzing the product on a polyacrylamide gel, it is possible to determine the transcriptional start site, as the length of the sequence on the gel represents the distance from the start site to the labeled primer.

The advanced polynucleotide technology described herein would overcome and resolve potential secondary structure encountered in RNA.

### F. Isothermal DNA Amplification

Isothermal DNA amplification may be performed as taught in U.S. Pat. No. 7,579,153 using the advanced polynucleotide technology described herein. Briefly, isothermal DNA amplification comprises the following steps: (i) providing a double stranded DNA having a hairpin at one end, the polynucleotide at the other end, and disposed therebetween a promoter sequence oriented so that synthesis by an RNA polymerase recognizing the promoter sequence proceeds in the direction of the hairpin; (ii) transcribing the double stranded DNA with an RNA polymerase that recognizes the promoter sequence to form an RNA transcript comprising copies of the promoter sequence and the polynucleotide; (iii) generating a complementary DNA from the RNA transcript; (iv) displacing a 5′ end of the RNA transcript from the complementary DNA so that the hairpin is reconstituted; and (v) extending the hairpin to generate the double stranded DNA containing a reconstituted promoter sequence, the RNA polymerase recognizing the reconstituted promoter sequence and synthesizing RNA transcripts. In a preferred embodiment, the step of generating includes forming a heteroduplex of said complementary DNA and said RNA transcript and wherein said step of displacing includes treating the heteroduplex with a helicase.

### G. Fluorescence In Situ Hybridization (FISH)

The advanced polynucleotide technology described herein can also be used to practice FISH. FISH is a cytogenetic technique used to detect and localize the presence or absence of specific DNA sequences on chromosomes. FISH uses fluorescent probes that bind to only those parts of the chromosome with which they show a high degree of sequence similarity. Fluorescence microscopy can be used to find out where the fluorescent probe bound to the chromosomes. FISH is often used for finding specific features in DNA for use in genetic counseling, medicine, and species identification. FISH can also be used to detect and localize specific mRNAs within tissue samples. In this context, it can help define the spatial-temporal patterns of gene expression within cells and tissues.

### H. Ligation Probes

The advanced polynucleotide technology described herein can also be used to practice multiplex PCR using ligation probes. Ligation probe methods are known to those of skill in the art. Briefly, ligation probes consist of two separate oligonucleotides, each containing a PCR primer sequence. It is only when these two hemi probes are both hybridized to their adjacent targets that they can be ligated. Only ligated probes will be amplified exponentially in a PCR. The number of probe ligation products therefore depends on the number of target sequences in the sample.

In some embodiments, two ligation probes are separated by about 1 to about 500 nucleotides, and prior to ligation the first probe is extended by a DNA thermostable polymerase lacking strand-displacement activity. A DNA thermostable polymerase lacking strand-displacement activity includes, but is not limited to, a Pfu polymerase. When the extended strand reaches the 5′-phosphate group of the second ligation probe, polymerization stops and a nick is created. This nick can be sealed by a thermostable ligase present in the reaction mixture, allowing for the entire reaction to occur in a single-reaction format.

### I. Next Generation Sequencing (NGS)

The polynucleotide combinations of the present disclosure may also be used in NGS applications. Instead of sequencing by sequential ligation of DNA probes, the primer combinations disclosed herein can be used in sequential hybridization without ligation. For a review of NGS technology, see Morozova et al., Genomics 92(5): 255-64, 2008, incorporated herein by reference in its entirety. NGS is readily understood and practiced by those of ordinary skill in the art.

### J. Insertion/Deletion (Indel) Detection

In some embodiments, and as described herein, use of a polynucleotide primer combination of the disclosure is used to detect a mutation in a target polynucleotide versus a non-target polynucleotide. In various aspects, the mutation is due to a deletion and in further aspects, the mutation is due to an insertion. Deletions and insertions are found in the normal human population, but they are also associated with disease and are frequently associated with human cancers.

The disclosure provides methods to detect insertions and/or deletions in a target polynucleotide using a polynucleotide primer combination as described herein. Quantitative PCR detection of low copy number alleles carrying a deletion or an insertion in the presence of high copy number wild type (i.e., normal) DNA is a comparable or more challenging task than detection of a single base mutation (i.e., substitution). Use of the polynucleotide primer combinations of the disclosure, however, affords an efficient way to detect these types of mutations. Detection of small deletions by polynucleotide primer combinations of the disclosure and qPCR is similar to detection of base substitutions. The forward polynucleotide primer combination is designed such that the approximately 6-12 base domain (f) is complementary to the proximal and distal sequences flanking the deletion in a target polynucleotide and where the deletion breakpoint is located in the middle of domain (f). Domain (f) can efficiently prime and promote a primer-extension reaction on the target polynucleotide due to formation of an uninterrupted stable duplex between domain (f) and target polynucleotide but cannot prime and promote a primer-extension reaction on a non-target polynucleotide due to an unstable interrupted duplex formed by domain (f) and the non-target polynucleotide. Specificity of the deletion-specific primer combination to the target polynucleotide can be further increased by adding a blocking oligonucleotide complementary to the non-target polynucleotide that will outcompete binding by domain (f) whose complement is not contiguous with the non-target polynucleotide. The 3′ end of the blocker polynucleotide has a group that prevents its extension by a DNA polymerase (i.e., an extension blocker). As a result, when combined with a reverse primer, a probe and a thermostable polymerase, the polynucleotide primer combination can selectively amplify and detect a small amount of DNA carrying a small deletion in the presence of large amount of non-target DNA in a quantitative PCR mode.

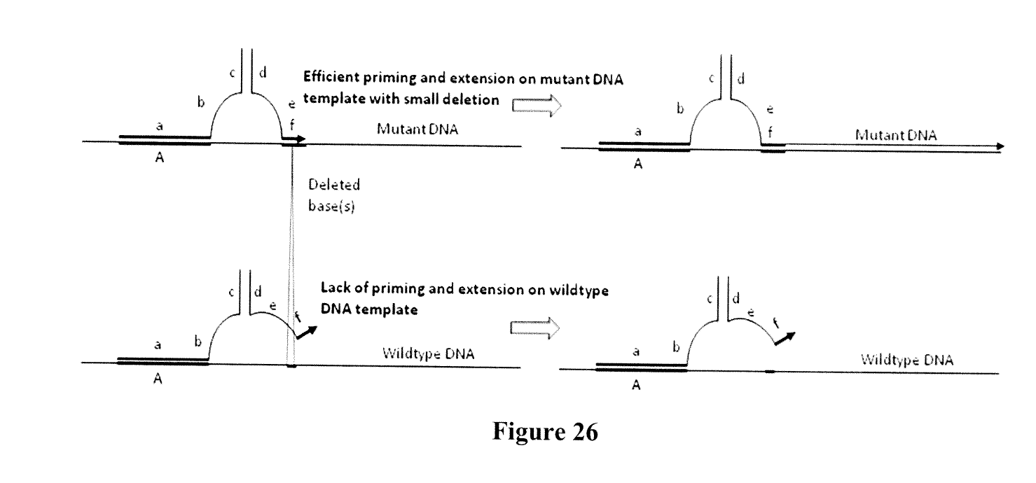

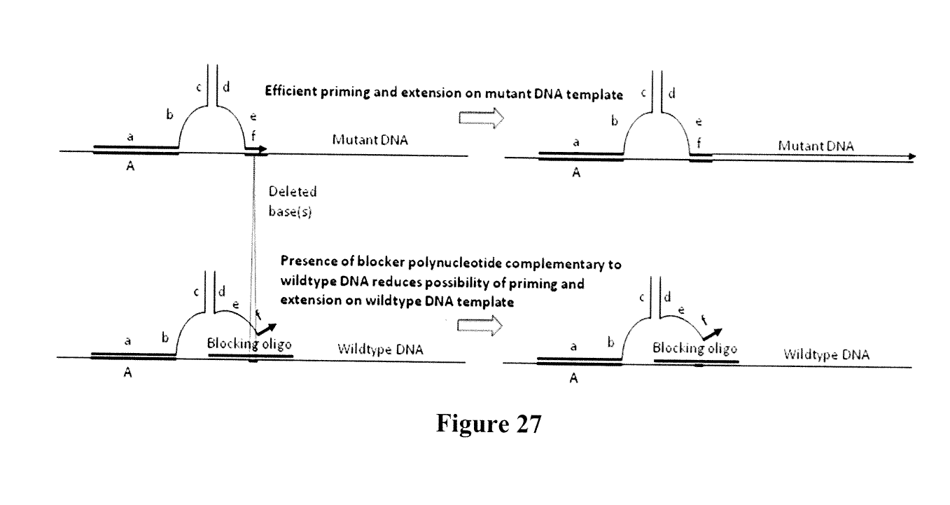

Accordingly, in various embodiments, for a relatively small deletion of DNA in a target polynucleotide (i.e., at least 1, 2, 3, 4, 5, 6, 7, 8, 9 or 10 nucleotides) it is contemplated that the sequence of domain (f) is identical to a mutant target polynucleotide wherein up to about 10 nucleotides are deleted relative to the wild type (non-target) polynucleotide (FIG. 26). In further aspects, a blocker polynucleotide is used to reduce the priming and extension of a non-target polynucleotide by specifically hybridizing to the non-target polynucleotide (FIG. 27).

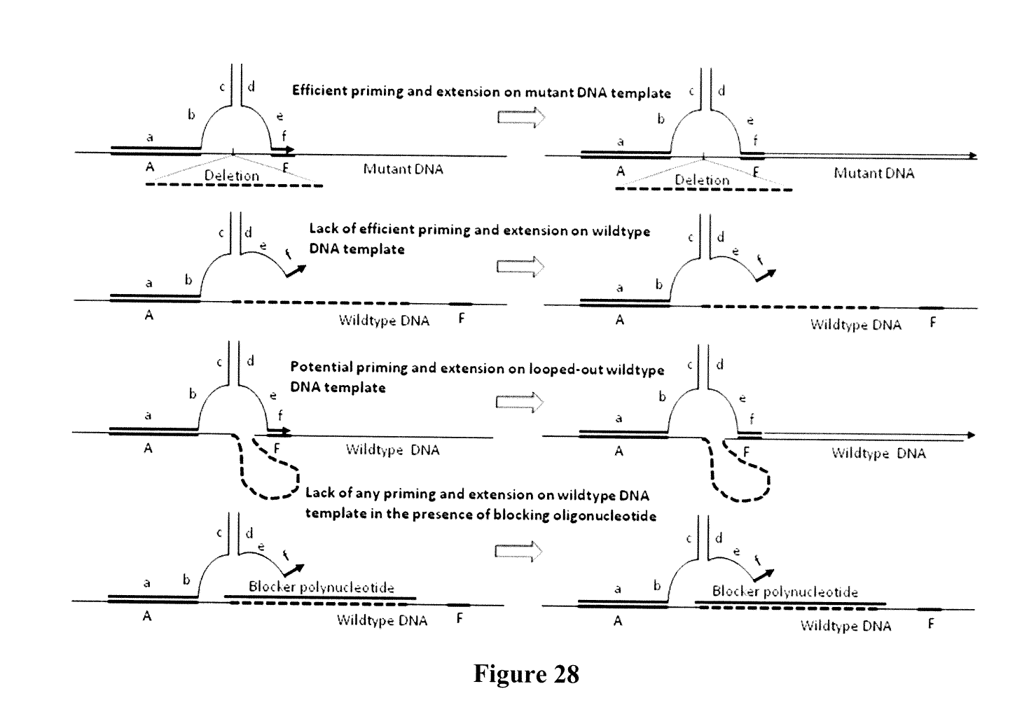

In further embodiments, for a larger deletion of DNA in a target polynucleotide (i.e., more than about 10 nucleotides) (FIG. 28). Detection of larger deletions by a polynucleotide primer combination of the disclosure and qPCR is different from detection of base substitutions or small deletions. In the case of a large deletion, the forward polynucleotide primer combination is designed such that the approximately 6-12 base domain (f) is complementary to both the target polynucleotide region (F) and the non-target polynucleotide region (F*) flanking the 5′-distal breakpoint of the deletion. Domain (f) can efficiently prime and promote a primer-extension reaction on the target polynucleotide due to formation of a stable complex between domain (f) and the target polynucleotide but cannot with the same efficiency prime and promote a primer-extension reaction on a non-target polynucleotide due to the distance between regions (A) and (F). However, priming of a non-target polynucleotide cannot be excluded completely for relatively short deletions (for example and without limitation, approximately 8-50 bases) due to formation of complexes with looped-out non-target polynucleotide templates. Such looping-out can be efficiently reduced or completely precluded by hybridization of a blocker polynucleotide comprising the deleted region that can extend through region (F) (see FIG. 28). The higher rigidity of double-stranded DNA formed within the putative deletion region will prevent loop formation, thus preventing domain (f) binding and primer extension on a non-target polynucleotide. The 3′ end of the blocker polynucleotide has a group that prevents its extension by a DNA polymerase (i.e., an extension blocker). As a result, when combined with a reverse polynucleotide primer, a probe and a thermostable polymerase, the polynucleotide primer combination can selectively amplify and detect a small amount of DNA carrying a larger deletion in the presence of a large amount of non-target polynucleotide in a quantitative PCR mode. The method described above does not require a precise, base resolution knowledge of the deletion boundaries. For this reason the method can be used for the simultaneous (multiplexed) detection of several potential deletions localized within a certain genomic site, each with distinct breakpoint s. The multiplexed method may utilize one first polynucleotide (1) or require several different first polynucleotides (1).

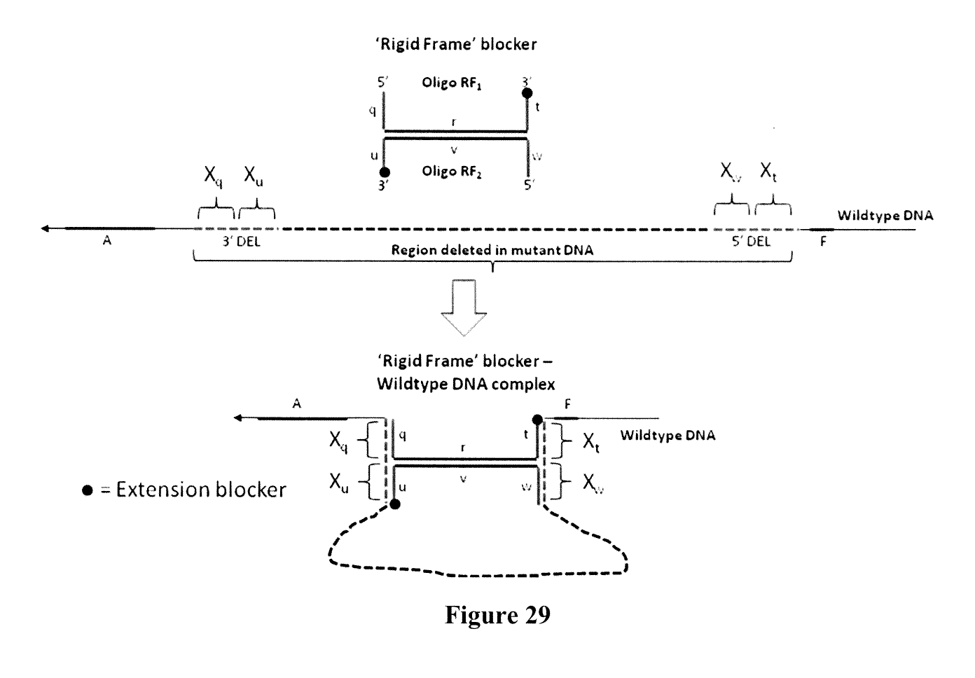

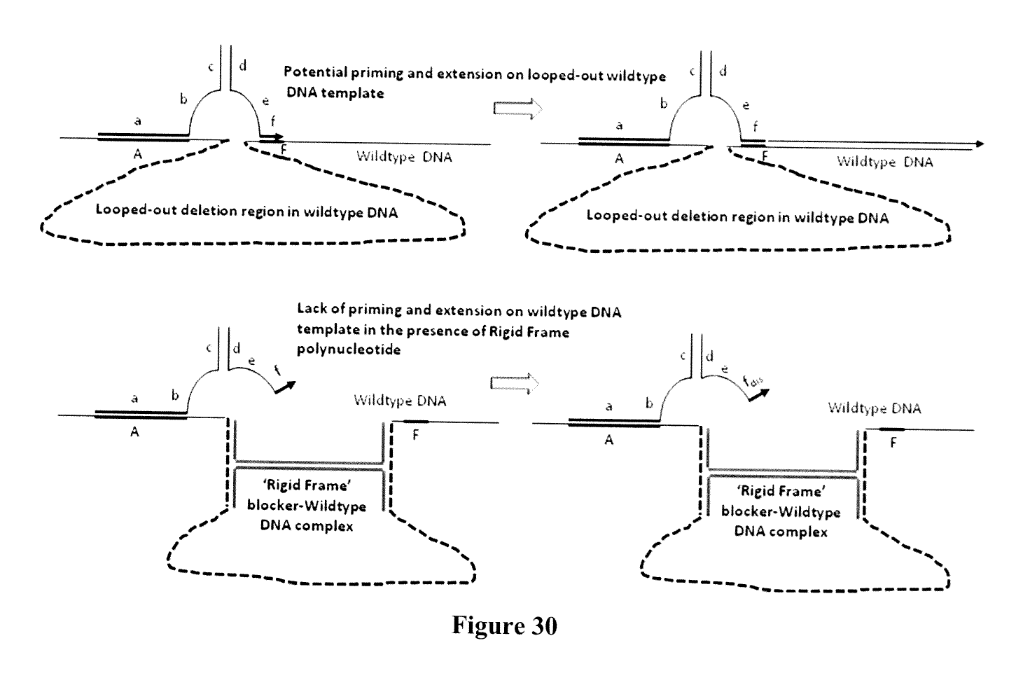

In still further embodiments, a polynucleotide primer combination of the disclosure further comprises a rigid frame blocker polynucleotide (RF). FIG. 29 provides a depiction of a rigid frame blocker polynucleotide. A rigid frame blocker polynucleotide is formed by hybridization of two polynucleotides RF1 and RF2 with complementary internal sequences (r) and (v). These sequences form an approximate 30-50 base-pair double-stranded stem, while terminal sequences q, t, u and v remain single-stranded. Sequences q and u are designed to be complementary to the 3′ end of the deleted region (“3′ DEL” in FIG. 29), sequences t and w are complementary to the 5′ end of the deleted region (“5′ DEL” in FIG. 29). The 3′ ends of the polynucleotides RF1 and RF2 have a group that prevents their extension by a DNA polymerase (i.e., an extension blocker). Interaction of the rigid frame blocker polynucleotides with wildtype (i.e., non-target) DNA (FIG. 30) results in the formation of a “Rigid Frame” blocker-wildtype DNA complex where regions (A) and (F) are separated by a distance sufficient to prevent simultaneous binding of domain (a) and domain (f) of the polynucleotide primer combination to regions (A) and (F), respectively, and extension of domain (f) by a DNA polymerase.

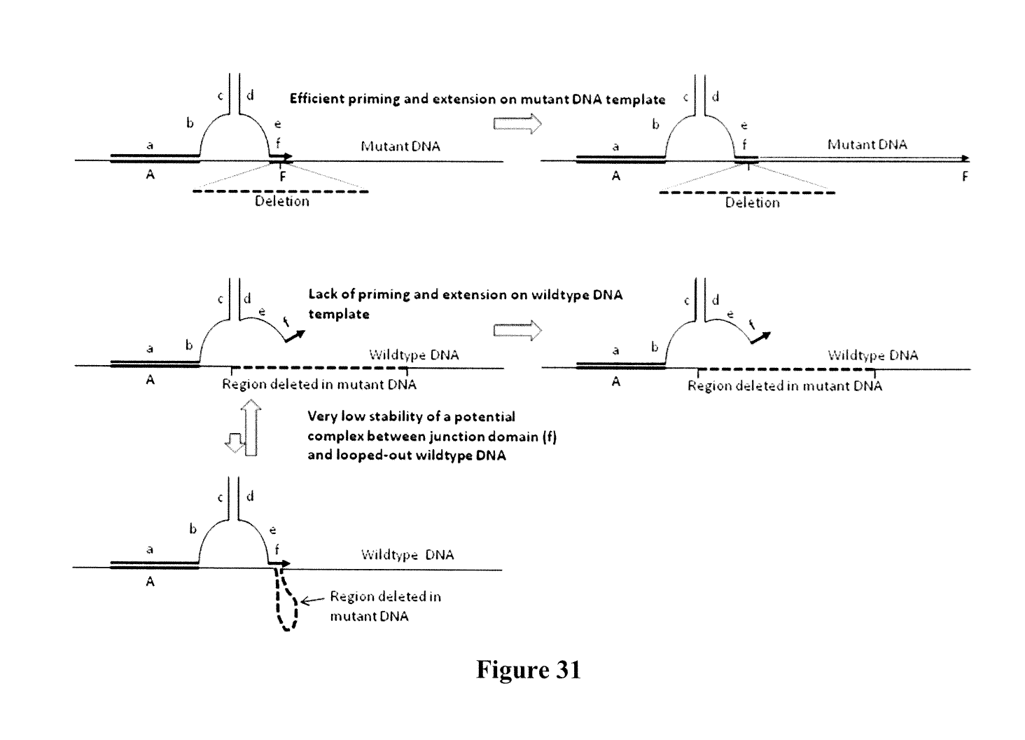

In another embodiment, a polynucleotide primer combination of the disclosure is used to detect a deletion in a target polynucleotide, wherein the second polynucleotide (2) comprises a junction-specific domain (f). Detection of large deletions by a polynucleotide primer combination of the disclosure and qPCR is achieved using a forward primer designed such that the 6-12 base priming domain (f) is complementary to the junction region formed by DNA sequences flanking the deleted fragment with the junction located in the middle or close to the middle of the priming domain (f) (for example and without limitation, if domain (f) is 8 nucleotides in length, then the junction is located at base number 3, 4, 5, or 6 from the 3′ end of domain (f)). Domain (f) can efficiently prime and promote a primer-extension reaction on the mutant (deleted) DNA due to formation of a stable complex between domain (f) and the mutant DNA but cannot prime and promote a primer-extension reaction on wildtype DNA due to a lack of complementarity between the wildtype DNA and 3′ portion of domain (f). Formation of a complex between domain (f) and looped-out wildtype DNA at high temperature (approximately 50°-70° C.) is unlikely due to very low stability of a such complex even at room temperature. Such looping-out of wildtype DNA can be further reduced or even completely precluded by hybridization of a blocker polynucleotide complementary to the deleted region. As a result of higher rigidity of double-stranded DNA formed within the putative deletion region of wildtype DNA, the loop formation will be prevented and any potential complex between domain (f) of the polynucleotide primer combination and wildtype DNA will not be formed and extended by DNA polymerase. As a result, when combined with a second reverse primer, a probe and a thermostable polymerase the polynucleotide primer combination can selectively amplify and detect a small amount of DNA carrying a long deletion in the presence of large amount of wildtype DNA in a quantitative PCR mode. The method depicted in FIG. 31 requires a precise, base resolution knowledge of the deletion boundaries. For this reason it would require several second polynucleotides (2) with different binding domains (f) that are complementary to corresponding deletion junction regions if used for the simultaneous (multiplexed) detection of several deletions localized within a certain genomic site. The multiplexed method may utilize one first polynucleotide (1) or require several different first polynucleotides (1).

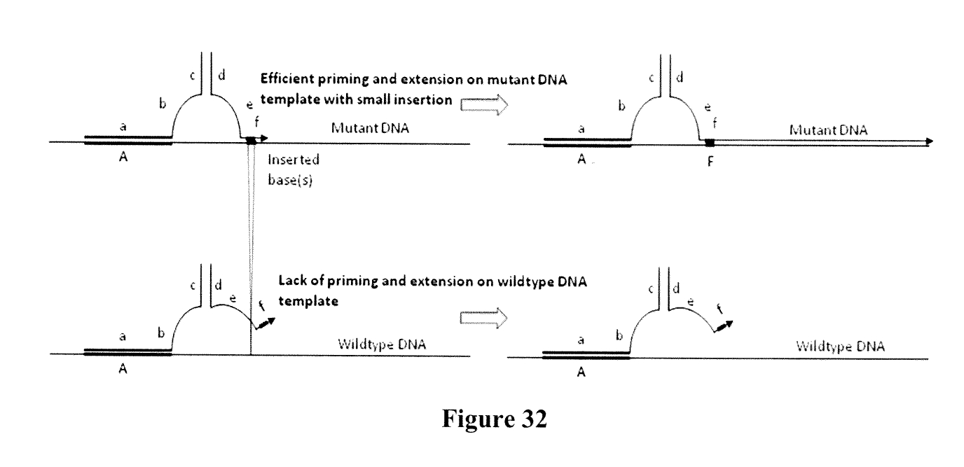

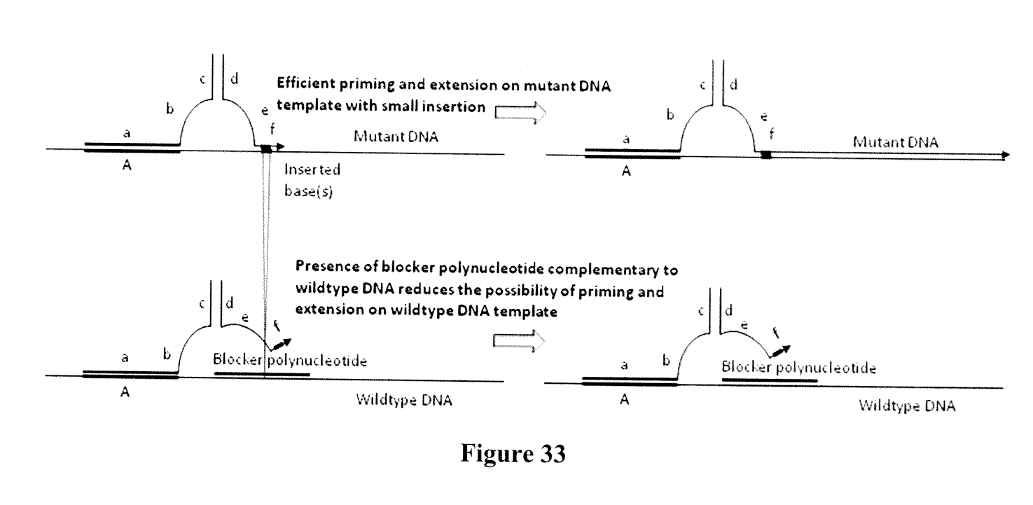

In some embodiments, a polynucleotide primer combination of the disclosure is used to detect an insertion of one or more nucleotides in a target polynucleotide (FIG. 32). Detection of small insertions by a polynucleotide primer combination of the disclosure and qPCR is similar to detection of base substitutions and small deletions. A forward primer is designed in such a way that the approximately 6-12 nucleotide priming domain (f) is complementary to the mutated region and the insertion is located in the middle of the priming domain (f). Domain (f) can efficiently prime and promote a primer-extension reaction on the mutant (inserted) DNA due to formation of a stable uninterrupted duplex between domain (f) and the mutant DNA but cannot prime and promote a primer-extension reaction on wildtype DNA due to instability of the interrupted duplex formed by domain (f) and wildtype DNA. Specificity of the insertion-specific polynucleotide primer combination to the mutant template can be further increased by adding a blocker polynucleotide complementary to the wild DNA template that will outcompete priming domain (f) for binding to wildtype DNA (see FIG. 33). The 3′ end of the blocker polynucleotide has a group that prevents its extension by a DNA polymerase (i.e., an extension blocker). As a result, when combined with a reverse primer, a probe and a thermostable polymerase, the polynucleotide primer combination can selectively amplify and detect a small amount of DNA carrying a small insertion in the presence of large amount of wildtype DNA in a quantitative PCR mode.

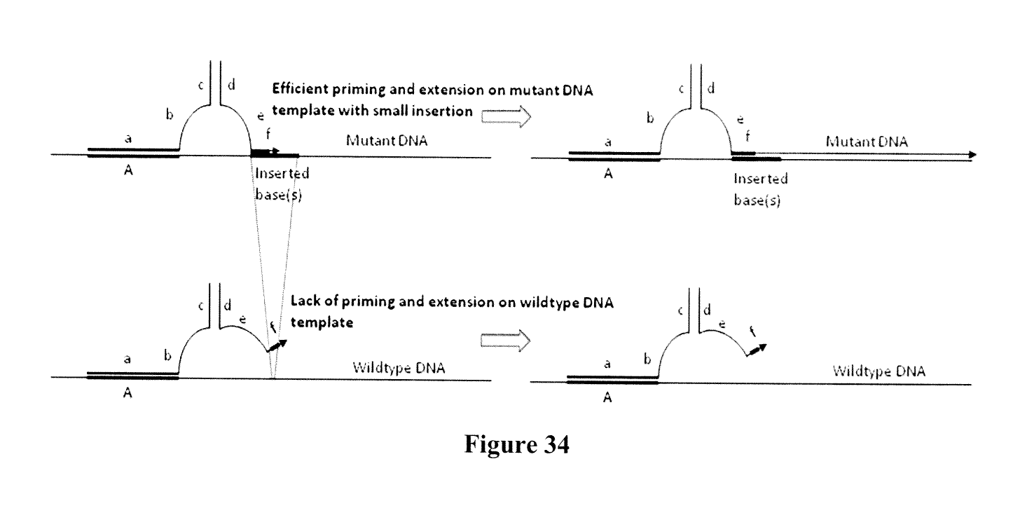

In an embodiment, a polynucleotide primer combination of the disclosure is used to detect a large insertion (at least about 8 nucleotides in length) (FIG. 34). In some aspects, domain (f) of the polynucleotide primer combination is designed to be complementary to the 3′ end of the inserted region. Domain (f) forms a stable complex with mutant DNA and becomes extended by a DNA polymerase, but domain (f) does not form a complex with wildtype DNA and cannot be extended by a DNA polymerase. As a result, when combined with a reverse primer, a probe and a thermostable polymerase, the polynucleotide primer combination can selectively amplify and detect a small amount of DNA carrying a large insertion in the presence of large amount of wildtype DNA in a quantitative PCR mode.

### X. Enzymes

In some aspects of any of the methods, the extension is performed by an enzyme that is capable of synthesizing a nucleic acid is quantitated in real-time. The enzymes useful in the practice of the disclosure include but are not limited to a DNA polymerase (which can include a thermostable DNA polymerase, e.g., a Taq DNA polymerase), RNA polymerase, and reverse transcriptase. Non-limiting examples of enzymes that may be used to practice the present disclosure include but are not limited to Deep VentR™ DNA Polymerase, LongAmp™ Taq DNA Polymerase, Phusion™ High-Fidelity DNA Polymerase, Phusion™ Hot Start High-Fidelity DNA Polymerase, VentR® DNA Polymerase, DyNAzyme™ II Hot Start DNA Polymerase, Phire™ Hot Start DNA Polymerase, Phusion™ Hot Start High-Fidelity DNA Polymerase, Crimson LongAmp™ Taq DNA Polymerase, DyNAzyme™ EXT DNA Polymerase, LongAmp™ Taq DNA Polymerase, Phusion™ High-Fidelity DNA Polymerase, Phusion™ Hot Start High-Fidelity DNA Polymerase, Taq DNA Polymerase with Standard Taq (Mg-free) Buffer, Taq DNA Polymerase with Standard Taq Buffer, Taq DNA Polymerase with ThermoPol II (Mg-free) Buffer, Taq DNA Polymerase with ThermoPol Buffer, Crimson Taq™ DNA Polymerase, Crimson Taq™ DNA Polymerase with (Mg-free) Buffer, Phire™ Hot Start DNA Polymerase, Phusion™ High-Fidelity DNA Polymerase, VentR® DNA Polymerase, VentR® (exo-) DNA Polymerase, Phire™ Hot Start DNA Polymerase, Phusion™ High-Fidelity DNA Polymerase, Phusion™ Hot Start High-Fidelity DNA Polymerase, Hemo KlenTaq™, Deep VentR™ (exo-) DNA Polymerase, Deep VentR™ DNA Polymerase, DyNAzyme™ EXT DNA Polymerase, Hemo KlenTaq™, LongAmp™ Taq DNA Polymerase, Phusion™ High-Fidelity DNA Polymerase, ProtoScript® AMV First Strand cDNA Synthesis Kit, ProtoScript® M-MuLV First Strand cDNA Synthesis Kit, Bst DNA Polymerase, Full Length, Bst DNA Polymerase, Large Fragment, Taq DNA Polymerase with ThermoPol Buffer, 9° Nm DNA Polymerase, Crimson Taq™ DNA Polymerase, Crimson Taq™ DNA Polymerase with (Mg-free) Buffer, Deep VentR™ (exo-) DNA Polymerase, Deep VentR™ DNA Polymerase, DyNAzyme™ EXT DNA Polymerase, DyNAzyme™ II Hot Start DNA Polymerase, Hemo KlenTaq™, Phusion™ High-Fidelity DNA Polymerase, Phusion™ Hot Start High-Fidelity DNA Polymerase, Sulfolobus DNA Polymerase IV, Therminator™ y DNA Polymerase, Therminator™ DNA Polymerase, Therminator™ II DNA Polymerase, Therminator™ III DNA Polymerase, VentR® DNA Polymerase, VentR® (exo-) DNA Polymerase, Bsu DNA Polymerase, Large Fragment, DNA Polymerase I (E. coli), DNA Polymerase I, Large (Klenow) Fragment, Klenow Fragment (3′→5′ exo-), phi29 DNA Polymerase, T4 DNA Polymerase, T7 DNA Polymerase (unmodified), Terminal Transferase, Reverse Transcriptases and RNA Polymerases, E. coli Poly(A) Polymerase, AMV Reverse Transcriptase, M-MuLV Reverse Transcriptase, phi6 RNA Polymerase (RdRP), Poly(U) Polymerase, SP6 RNA Polymerase, and T7 RNA Polymerase.

### XI. Labels

In some aspects, the second polynucleotide comprises a label. In other aspects, any polynucleotide used in the methods described herein comprises a label. In some of these aspects the label is fluorescent. Methods of labeling oligonucleotides with fluorescent molecules and measuring fluorescence are well known in the art. Fluorescent labels useful in the practice of the disclosure include but are not limited to 1,8-ANS (1-Anilinonaphthalene-8-sulfonic acid), 1-Anilinonaphthalene-8-sulfonic acid (1,8-ANS), 5-(and-6)-Carboxy-2′,7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), BO-PRO-1-DNA, BO-PRO-3-DNA, BOBO-1-DNA, BOBO-3-DNA, BODIPY 650/665-X, MeOH, BODIPY FL conjugate, BODIPY FL, MeOH, Bodipy R6G SE, BODIPY R6G, MeOH, BODIPY TMR-X antibody conjugate pH 7.2, Bodipy TMR-X conjugate, BODIPY TMR-X, MeOH, BODIPY TMR-X, SE, BODIPY TR-X phallacidin pH 7.0, BODIPY TR-X, MeOH, BODIPY TR-X, SE, BOPRO-1, BOPRO-3, Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, Cy 5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, DiI, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, Ethidium Bromide, Ethidium homodimer, Ethidium homodimer-1-DNA, eYFP (Enhanced Yellow Fluorescent Protein), FDA, FITC, FITC antibody conjugate pH 8.0, FlAsH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, high Ca, Fura-2, no Ca, GFP(S65T), HcRed, Hoechst 33258, Hoechst 33258-DNA, Hoechst 33342, Indo-1 Ca2+, Indo-1, Ca free, Indo-1, Ca saturated, JC-1, JC-1 pH 8.2, Lissamine rhodamine, LOLO-1-DNA, Lucifer Yellow, CH, LysoSensor Blue, LysoSensor Blue pH 5.0, LysoSensor Green, LysoSensor Green pH 5.0, LysoSensor Yellow pH 3.0, LysoSensor Yellow pH 9.0, LysoTracker Blue, LysoTracker Green, LysoTracker Red, Magnesium Green, Magnesium Green Mg2+, Magnesium Orange, Marina Blue, mBanana, mCherry, mHoneydew, MitoTracker Green, MitoTracker Green FM, MeOH, MitoTracker Orange, MitoTracker Orange, MeOH, MitoTracker Red, MitoTracker Red, MeOH, mOrange, mPlum, mRFP, mStrawberry, mTangerine, NBD-X, NBD-X, MeOH, NeuroTrace 500/525, green fluorescent Nissl stain-RNA, Nile Blue, EtOH, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, PO-PRO-1, PO-PRO-1-DNA, PO-PRO-3, PO-PRO-3-DNA, POPO-1, POPO-1-DNA, POPO-3, Propidium Iodide, Propidium Iodide-DNA, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodaminen Green pH 7.0, Rhodol Green antibody conjugate pH 8.0, Sapphire, SBFI-Na+, Sodium Green Na+, Sulforhodamine 101, SYBR Green I, SYPRO Ruby, SYTO 13-DNA, SYTO 45-DNA, SYTOX Blue-DNA, Tetramethylrhodamine antibody conjugate pH 8.0, Tetramethylrhodamine dextran pH 7.0, Texas Red-X antibody conjugate pH 7.2, TO-PRO-1-DNA, TO-PRO-3-DNA, TOTO-1-DNA, TOTO-3-DNA, TRITC, X-Rhod-1 Ca2+, YO-PRO-1-DNA, YO-PRO-3-DNA, YOYO-1-DNA, and YOYO-3-DNA.

Other labels besides fluorescent molecules can be used, such as chemiluminescent molecules, which will give a detectable signal or a change in detectable signal upon hybridization, and radioactive molecules.

In some embodiments, the second polynucleotide comprises a quencher that attenuates the fluorescence signal of a label. In other embodiments, the fourth polynucleotide comprises a quencher that attenuates the fluorescence signal of a label. Quenchers contemplated for use in practice of the methods of the disclosure include but are not limited to Black Hole Quencher 1, Black Hole Quencher-2, Iowa Black FQ, Iowa Black RQ, Zen quencher, a G-Base, and Dabcyl.

The references cited herein throughout, to the extent that they provide exemplary procedural or other details supplementary to those set forth herein, are all specifically incorporated herein by reference.

### EXAMPLES

A person of skill in the art will appreciate that when primers or primer combinations are referred to as being in “forward” or “reverse” orientations, these designations are arbitrary conventions used in describing polymerase chain reactions (PCR) and the structural relationship of the primers and the template. Thus, as is apparent to a person of skill in the art, re-orienting a PCR schematic diagram by flipping it 180° would result in “forward” primers becoming “reverse” primers and “reverse” primers becoming “forward” primers, and as such, designation of, for example, one primer combination as a forward primer or a reverse primer is not a limitation on the structure or use of that particular primer combination.

### Example 1

### Real Time PCR Using Primer Combinations of the Disclosure where Region (F) is 5′ to Region (A) in the Target Polynucleotide**

### Materials

Wild Type Human Genomic DNA from Promega #G1471

Synthetic KRAS templates: seven synthetic KRAS mutant DNA clones have been generated by GenScript. 610 bp fragment of the synthetized mutant DNA corresponding to 10270-10879 bp of human KRAS gene (NG 007524) was subcloned at EcoRV site of pUC57 vector.

Template DNA: To generate KRAS mutant DNA templates for real time PCR, Promega WT genomic DNA was spiked with the designated amount of synthetic KRAS G12V genomic DNA so that number of mutant KRAS copies varied from one to 14,000 copies per 50 ng of total DNA, then template DNA was aliquoted and stored at −20° C. until use.

Real time qPCR mix: BioRad IQ Supermix #170-8862.

TABLE 1
Oligonucleotides used in PCR

| Mutation | Forward Primer | Fixer | Blocker |
|---|---|---|---|
| G12V | 10-374 | 10-307 | 10-395 |
| G12A | 10-403 | 10-307 | 10-395 |
| G12R | 10-402 | 10-440 | 10-394 |
| G12C | 10-401 | 10-307 | 10-395 |
| G12D | 10-399 | 10-307 | 10-395 |
| G12S | 10-380 | 10-307 | 10-395 |
| G13D | 10-386 | 10-307 | 10-215 |

Common Oligonucleotides:

Reverse primer: 10-208

Taqman probe: 10-210

TABLE 2
Oligonucleotide sequences

| | | SEQ |
|---|---|---|
| | | ID |
| Oligonucleotide | Sequence (5′→3′) | NO |
| 10-374 | CAGAGACGCAGGGATGAGAAGTTCTCTCTCTCTCAG | 1 |
| | CTGTTG | |
| 10-403 | CAGAGACGCAGGGATGAGAAGTTCTCTCTCTCTCTC | 2 |
| | TCAGCTGCTG | |
| 10-402 | CAGAGACGCAGGGATGAGAAGTTCTCTCTCTCTCTC | 3 |
| | TCGAGCTCGT | |
| 10-401 | CAGAGACGCAGGGATGAGAAGTTCTCTCTCTCTCTC | 4 |
| | TCGAGCTTGT | |
| 10-399 | CAGAGACGCAGGGATGAGAAGTTCTCTCTCTCTCTC | 5 |
| | TCAGCTGATG | |
| 10-380 | CAGAGACGCAGGGATGAGAAGTTCTCTCTCTCTCAG | 6 |
| | CTAGTG | |
| 10-386 | CAGAGACGCAGGGATGAGAAGTTCTCTCTCTCTCTC | 7 |
| | TCGGTGACGT | |
| 10-307 | ATTATAAGGCCTGCTGAAAATGACTGAATATAAACT | 8 |
| | CTCTCTCTACTTCTCATCCCTGCGTCTCTG/3Phos/ | |
| 10-440 | ATTATAAGGCCTGCTGAAAATGACTGAATATAAACT | 9 |
| | CCTCTCCAACACTTCTCATCCCTGCGTCTCTG/3Phos/ | |
| 10-395 | TTGGAGCTGGTGGCGTAGGC/3Phos/ | 10 |
| 10-394 | GTTGGAGCTGGTGGCGTAGGC/3Phos/ | 11 |
| 10-215 | GGTGGCGTAGGCAAGAGTGCC/3Phos/ | 12 |
| 10-208 | TTGTTGGATCATATTCGTCCACAAAATG | 13 |
| 10-210 | /56-FAM/AGCTGT ATC/ZEN/GTC AAG GCA CTCTTG | 14 |
| | CC/3IABkFQ/ | |

TABLE 3
Term designations

| Term | Designation |
|---|---|
| /3Phos/ | 3′ Phosphate group |
| /56-FAM/ | 5′ Fluorescein |
| /Zen/ | Internal ZEN quencher |
| /3IABkFQ/ | 3′ Iowa Black FQ quencher |

### Methods

Real-time amplification reaction: Amplification was carried out in triplicate with 25 μl aliquots consisting of 12.5 μl of 2× BioRad IQ Supermix, 200 nM of KRAS mutant specific second polynucleotide (2), 50 nM of first polynucleotide (1), 500 nM of blocker polynucleotide, 250 nM of probe 10-210, 200 nM of reverse primer 10-208 and 50 ng of template DNA using BioRad CFX96 Real Time System. Amplification was performed using the following thermal cycling profile: one cycle at 94° C. for 3 minutes, followed by 60 cycles at 94° C. for 10 seconds and 65° C. for 1 minute.

### Results

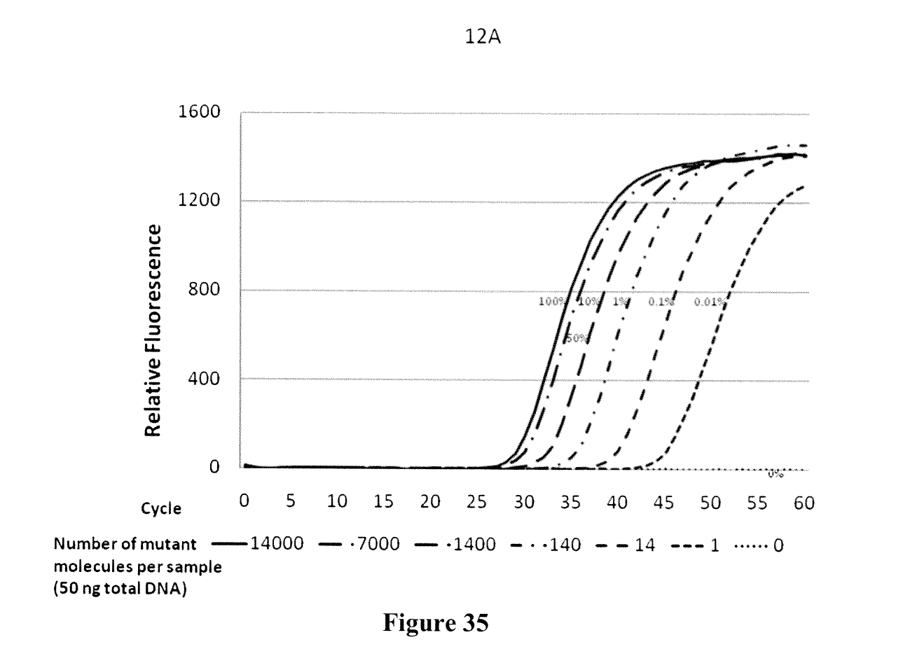

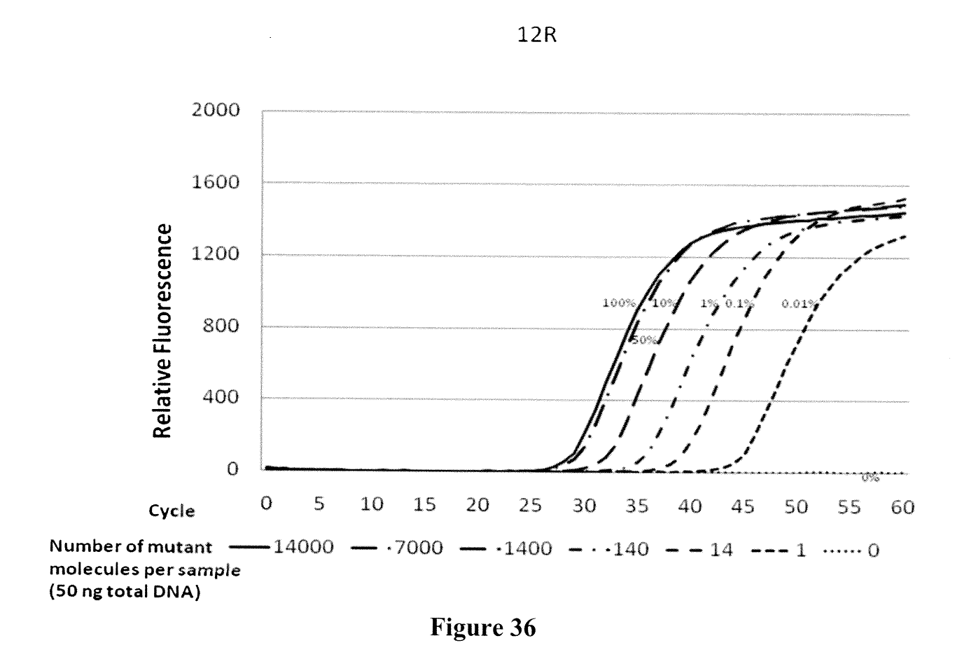

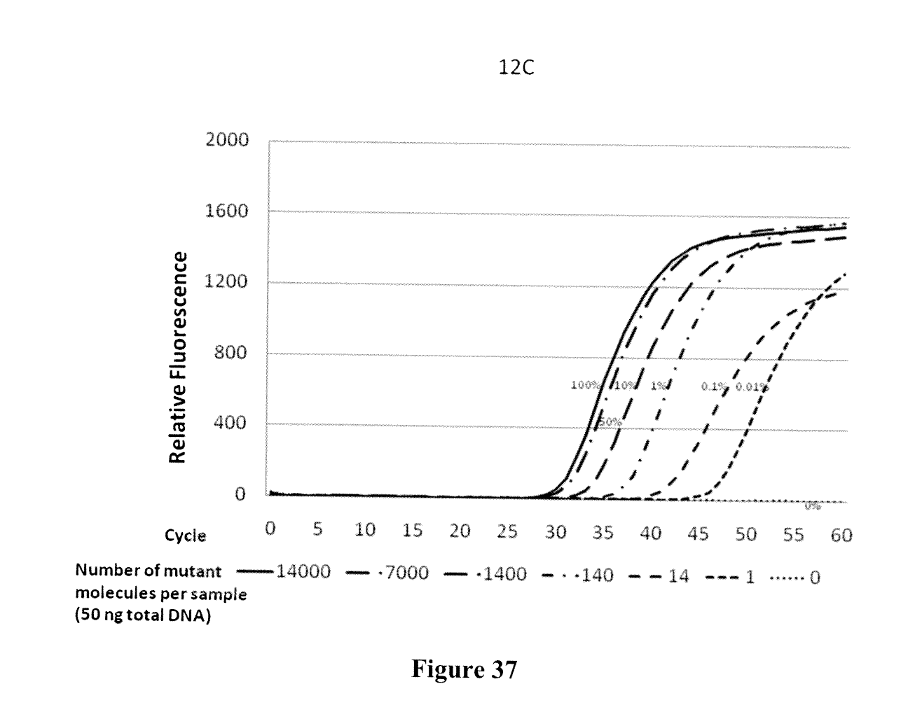

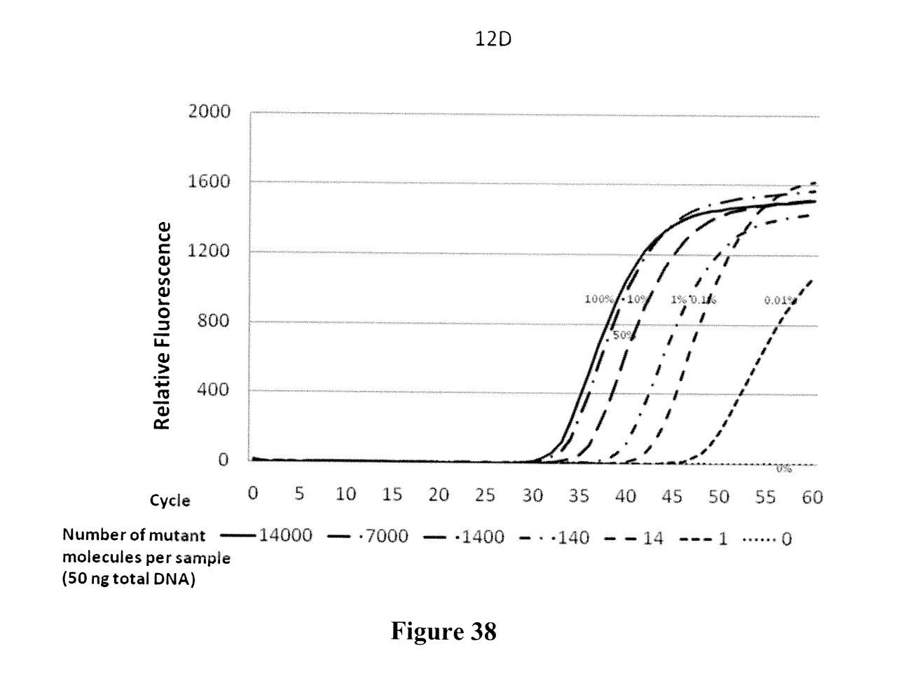

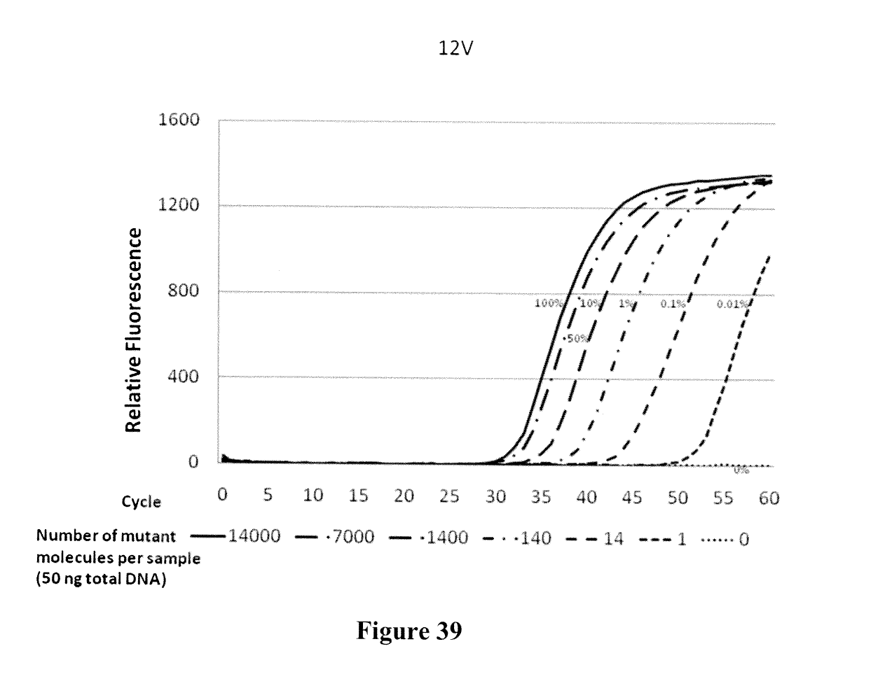

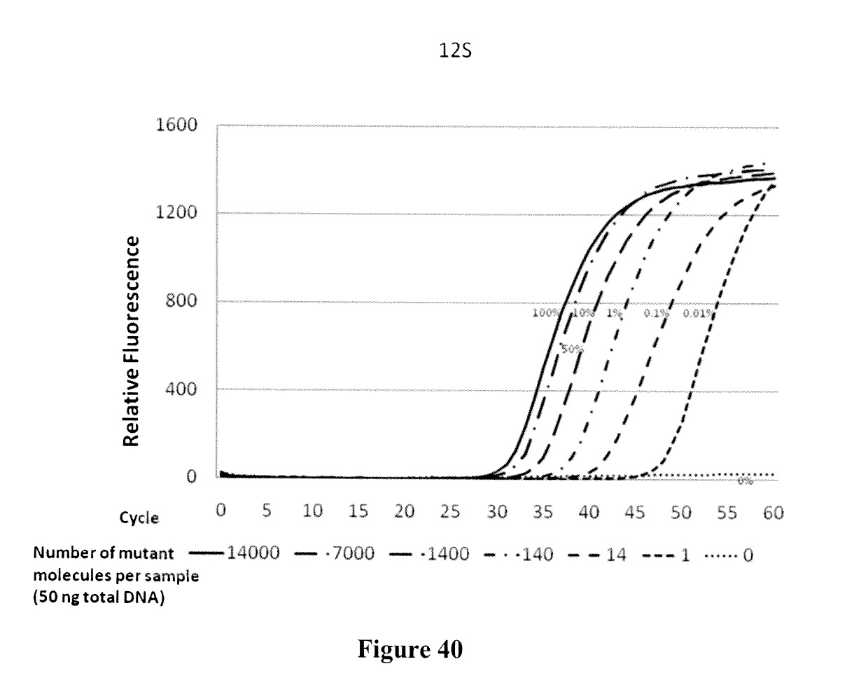

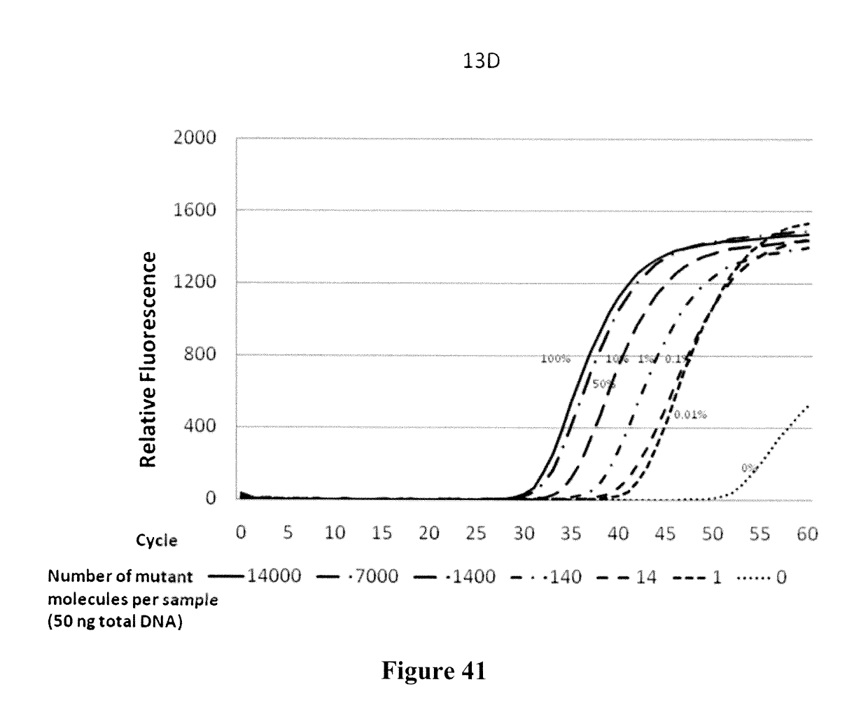

qPCR curves for 7 different KRAS mutant samples containing 100% (14,000 mutant DNA copies), 50% (7,000 mutant DNA copies), 10% (1,400 mutant DNA copies), 1% (140 mutant DNA copies), 0.1% (14 mutant DNA copies), 0.01% (1 mutant DNA copy), and 0% of mutant allele G12V are shown in FIGS. 35-41.

### Conclusions

Data above demonstrated sensitivity level of 0.01% or 1 mutant DNA molecule in a context of 14,000 copies of WT genomic DNA while maintaining absolute (zero background) selectivity for the six out of seven mutations. Such parameters of the assay satisfied the most demanding characteristics of a diagnostic assay including rare cancer cell detection and noninvasive diagnostic.

### Example 2

### Primer Combinations Specific to the KRAS G12V Mutation Demonstrate Allele Discrimination when Region (A) is 5′ to Region (F) in the Target Polynucleotide (Opposite Orientation Relative to Example 1)

### Materials

Oligonucleotides:

Second polynucleotide (2) G12V specific: 10-256, where the G12V specific base is at the 3′ terminal position of the priming domain and has a 1 base overlap with the WT specific blocking domain of the first polynucleotide (1).

First polynucleotide (1) WT KRAS specific blocking domain: 10-257, where the WT specific base is the 5′ terminal position of the blocking domain and has a 1 base overlap with the G12V specific priming domain (f).

Reverse primer: 10-208

Taqman probe: 10-210

TABLE 4
Oligonucleotide sequences:

| | | SEQ ID |
|---|---|---|
| Oligonucleotide | Sequence (5′→3′) | NO |
| 10-256 | CCGCGGCCCGGGCGCGGCCCGGCTTcTcTCTTccttc/idS | 15 |
| | p/tctccttccctcttggagctgt | |
| 10-257 | GTGGCGTAGGCAAGAGTGCCTTGACCTTTTTCTTTCT | 16 |
| | TTTTCTTTCTTTTCCGGGCCGCGCCCGGGCCGCGG/3P | |
| | hos/ | |
| 10-208 | TTGTTGGATCATATTCGTCCACAAAATG | 13 |
| 10-210 | /56-FAM/AGCTGT ATC/ZEN/GTC AAG GCA CTCTTG | 14 |
| | CC/3IABkFQ/ | |

TABLE 5
Term Designations

| Term | Designation |
|---|---|
| /idSp/ | internal stable abasic site |
| /3Phos/ | 3′ Phosphate group |
| /56-FAM/ | 5′ Fluorescein |
| /Zen/ | Internal ZEN quencher |
| /3IABkFQ/ | 3′ Iowa Black FQ quencher |

0.1×TE buffer: 10 mM Tris, 0.1 mM EDTA pH=8.0

Genomic DNA isolation kit: Qiagen DNeasy Blood & Tissue Kit #69504

Wild Type human genomic DNA template: Promega human genomic DNA #G1471

Mutant template: (G12V) genomic DNA isolated from freshly harvested SW480 colorectal adenocarcinoma cells (ATCC# CCL-228)

Real time SYBR Green qPCR mix: BioRad IQ SYBR Green Supermix #170-8882

### Methods

Genomic DNA Isolation:

KRAS G12V human genomic DNA was isolated from freshly harvested SW480 cells using Qiagen DNeasy Blood & Tissue Kit according to manufacturer protocol, resuspended in 0.1×TE buffer at a concentration of 100 ng/μl, aliquoted and stored at −20° C. until use.

Real-Time Amplification Reaction:

Amplification was carried out in triplicate with 25 μl aliquots consisting of 12.5 μl of 2× BioRad IQ Supermix, 200 nM of KRAS G12V specific second polynucleotide (2), 400 nM of WT KRAS specific first polynucleotide (1), 250 nM of probe 10-210, 200 nM of reverse primer 10-208 and 50 ng of template (WT or G12V mutant) DNA using BioRad CFX96 Real Time System. Amplification was performed using the following thermal cycling profile: one cycle at 94° C. for 3 minutes, followed by 60 cycles at 94° C. for 15 seconds and 65° C. for 1 minute 20 seconds.

### Results

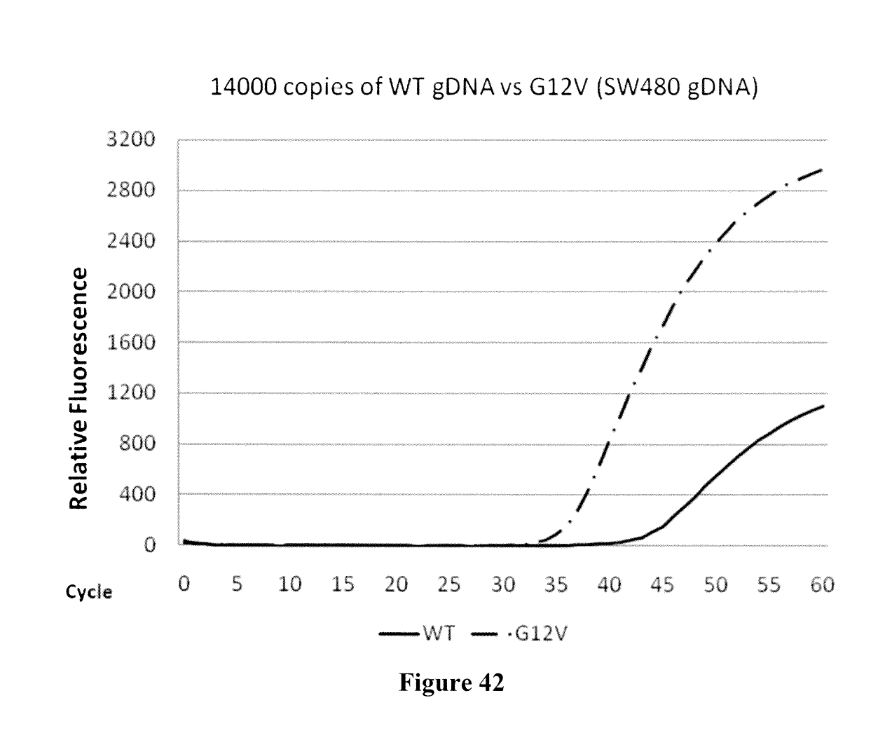

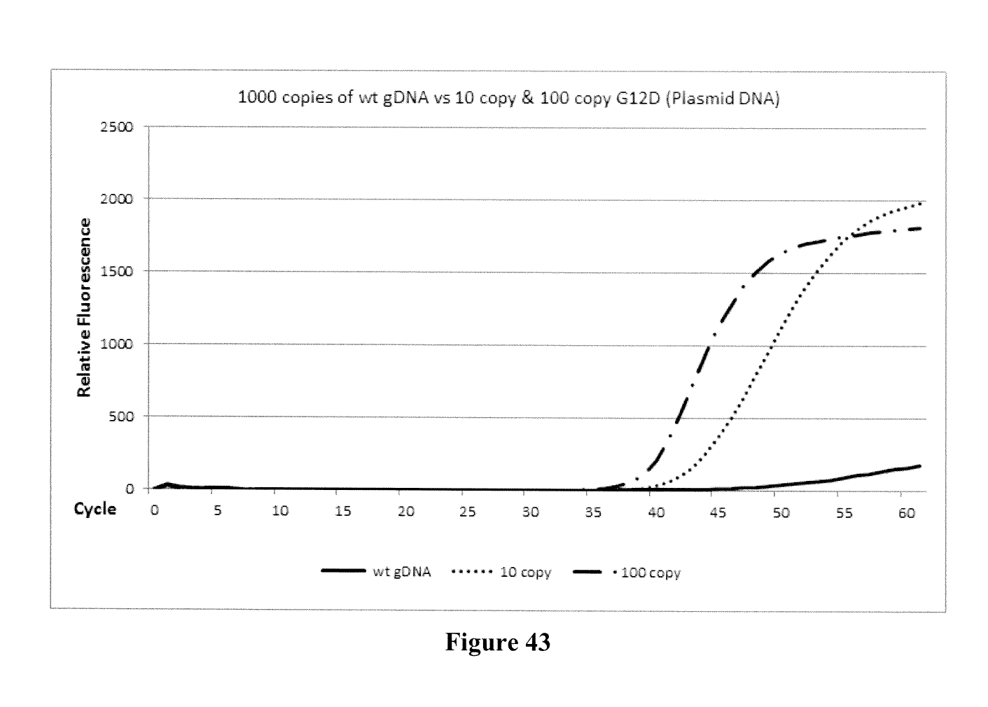

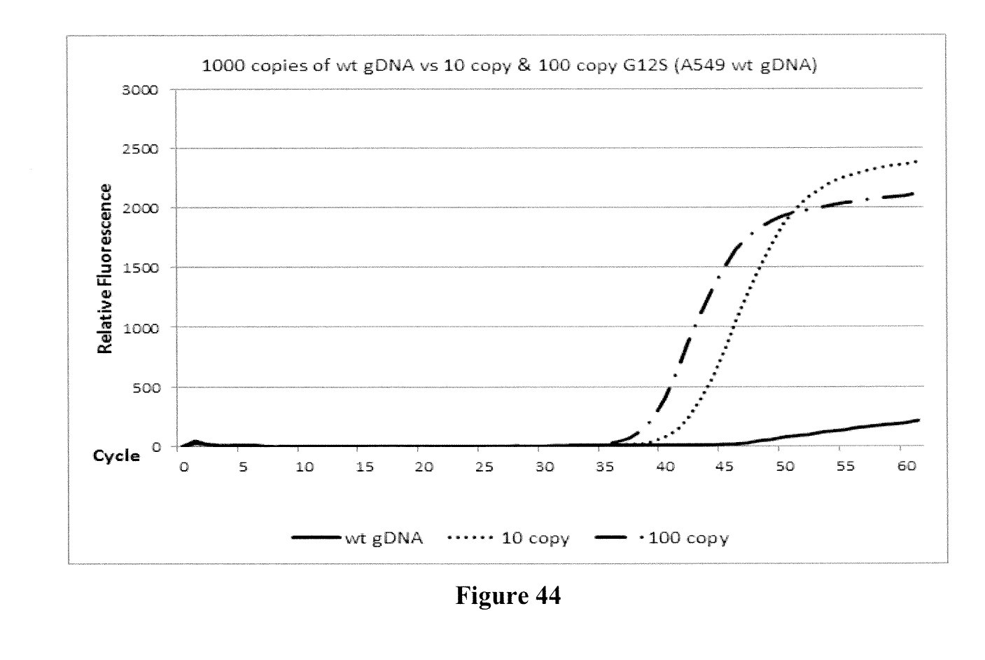

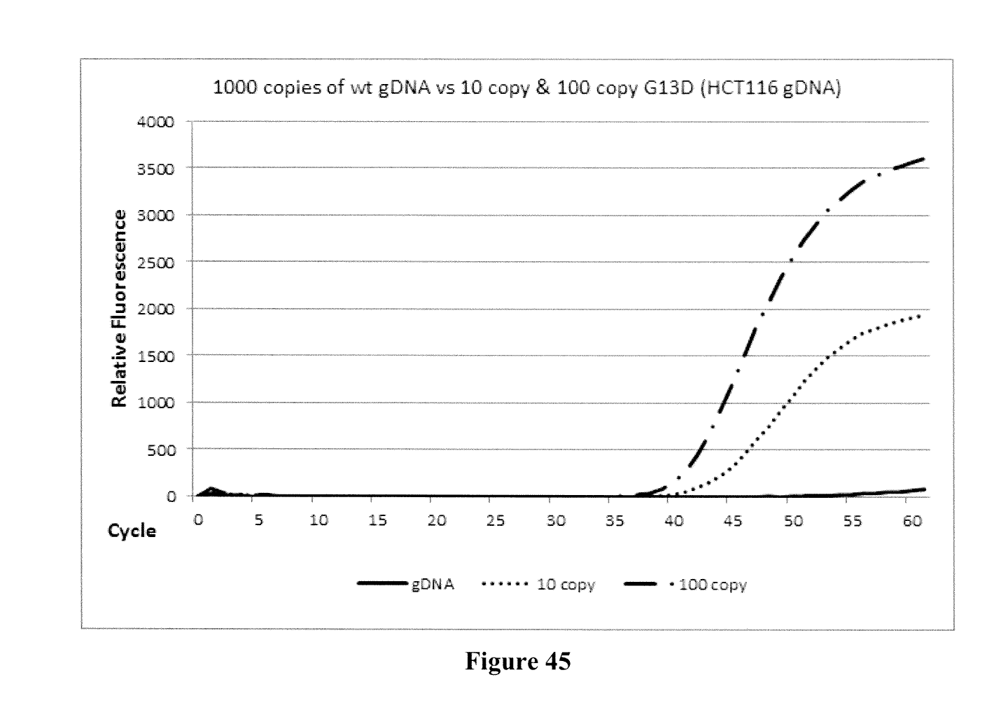

qPCR curves for G12V KRAS mutant sample containing 50 ng SW480 DNA (14,000 mutant DNA copies) versus 50 ng (14,000 copies) of WT genomic DNA are shown in FIG. 42. The delta Ct between the two templates was 9, indicating that the G12V-specific second polynucleotide (2) with WT-specific blocking tail (domain a_{5′}) of the first polynucleotide (1) demonstrates significant allele discrimination.

### Example 3

### Real Time PCR Using Primer Combinations of the Disclosure for KRAS G12S, G12D and G13D Mutations Wherein the Blocking Oligonucleotide not Only Overlaps Entirely with Region F* but Also Overlaps Either Partially or Entirely with Region A and Wherein Region b Of the First Polynucleotide can be Absent (i.e., Comprised of 0 Bases)

### Materials

Wild-Type Human Genomic DNA from Promega #G1471

Synthetic KRAS templates: G12D synthetic KRAS mutant DNA clones were generated by GenScript. A 610 bp fragment of the synthesized mutant DNA corresponding to bases 10270-10879 of human KRAS gene (GenBank Accession Number NG_{—}007524; SEQ ID NO: 17) was subcloned at EcoRV site of pUC57 vector. G12S mutant genomic DNA was obtained from A549 cells while G13D mutant genomic DNA was obtained from HCT116 cells.

Template DNA: To generate KRAS mutant DNA templates for real time PCR, Promega WT genomic DNA was spiked with the designated amount of synthetic KRAS G12S, G12D and G13D genomic DNA so that number of mutant KRAS copies varied from 10 to 100 copies per 3.6 nanograms (ng) of total DNA, then template DNA was aliquoted and stored at −20° C. until use.

Real time qPCR mix: Maxima probe qPCR master mix #K 0261

Common Oligonucleotides:

Reverse primer: 10-208

Taqman probe: 10-210.

### Methods

Real-time amplification reaction: Amplification was carried out in triplicate with 25 μl aliquots consisting of 12.5 μl of 2× Maxima probe qPCR master mix, 200 nM of KRAS mutant specific polynucleotide 12-147, 12-148 and 12-122, 50 nM of polynucleotide 10-244, 12-093 and 12-119, 500 nM of Blocking polynucleotide 12-196, 12-180 and 12-179, 250 nM of probe 10-210, 200 nM of reverse primer 10-208 and 3.6 nanograms of template DNA using BioRad CFX96 Real Time System. Amplification was performed using the following thermal cycling profile: one cycle at 95° C. for 10 minutes, followed by 60 cycles at 95° C. for 14 seconds and 65° C. for 1 minute.

### Results

qPCR curves for 3 different KRAS mutant samples containing 10 and 100 copies of KRAS G12S, G12D and G13D in a background of 3.6 ng of wild-type genomic DNA and a negative control curve showing background amplification from 3.6 ng of wild-type genomic DNA.

### Conclusions

Data described above demonstrated a sensitivity level of 1% or 10 mutant DNA molecules in a context of 1,000 copies of WT genomic DNA while maintaining absolute (i.e., zero background) selectivity for the 3 KRAS mutations. Such parameters of the assay satisfied the most demanding characteristics of a diagnostic assay including rare cancer cell detection and a noninvasive diagnostic.

TABLE 6
Oligonucleotides used in PCR

| Mutation | Forward Primer | Fixer | Blocker |
|---|---|---|---|
| G12S | 12-147 | 10-244 | 12-196 |
| G12D | 12-148 | 12-093 | 12-180 |
| G13D | 12-122 | 12-119 | 12-179 |

TABLE 7
Oligonucleotide sequences

| | | SEQ |
|---|---|---|
| Oligonu- | | ID |
| cleotide | Sequence (5′→3′) | NO |
| 12-147 | CAGAGACGCAGGGATGAGAAGT-tctctctc- | 18 |
| | AGCTAGTG | |
| 12-148 | CAGAGACGCAGGGATGAGAAGT-tctctctc- | 19 |
| | GCTGATGG | |
| 12-122 | CAGAGACGCAGGGATGAGAAGT-tctctctc- | 20 |
| | GGTGACGT | |
| 10-244 | CAGAGACGCAGGGATGAGAAGTTCTCTCTCTCTCT | 21 |
| | CTCGAGCTTGT/3Phos/ | |
| 12-093 | GCTGAAAATGACTGAATATAAACTTGTGGTAGTTG | 22 |
| | ACTTCTCATCCCTGCGTCTCTG/3Phos/ | |
| 12-119 | ATGACTGAATATAAACTTGTGGTAGTTGGAGCTAC | 23 |
| | TTCTCATCCCTGCGTCTCTG/3Phos/ | |
| 12-196 | AGTTGGAGCTGGTGGCGTAGGCA/3Phos/ | 24 |
| 12-180 | TAGTTGGAGCTGGTGGCGTAGGCAA/3Phos/ | 25 |
| 12-179 | TTGGAGCTGGTGGCGTAGGCAAGAG/3Phos/ | 26 |

TABLE 8
Term designations

| Term | Designation |
|---|---|
| /3Phos/ | 3′ Phosphate group |
| /56-FAM/ | 5′ Fluorescein |
| /Zen/ | Internal ZEN quencher |
| /3IABkFQ/ | 3′ Iowa Black FQ quencher |

1. (canceled)

2. A polynucleotide primer combination comprising a first polynucleotide (1) and a second polynucleotide (2),
the first polynucleotide (1) comprising a first domain (a) having a sequence that is sufficiently complementary to a first target polynucleotide region (A), a second domain (c) comprising a unique polynucleotide sequence, and a third domain (b) comprising a polymer sequence that is not sufficiently complementary to hybridize to a domain in the first polynucleotide (1), a domain in the second polynucleotide (2), or a domain in the target polynucleotide, wherein domains in the first polynucleotide are arranged 5′-a-b-c-3′;
the second polynucleotide (2) comprising a first domain (f) having a sequence that is sufficiently complementary to a sequence in a second target polynucleotide region (F), a second domain (d) comprising a polynucleotide sequence sufficiently complementary to (c) such that (c) and (d) will hybridize under appropriate conditions, and a third domain (e) comprising a polynucleotide sequence that is not sufficiently complementary to hybridize to a domain in the first polynucleotide (1), a domain in the second polynucleotide (2), or a domain in the target polynucleotide, wherein domains in the second polymer are arranged 5′-d-e-f-3′,
wherein under conditions in which region (A) specifically hybridizes to domain (a) and region (F) specifically hybridizes to domain (f), domain (c) hybridizes to domain (d) and neither domain (b) nor domain (e) hybridizes to a domain in the first polynucleotide (1), a domain in second polynucleotide (2) or a domain in the target polynucleotide.

3-6. (canceled)

7. The polynucleotide primer combination of claim 2, wherein the second polynucleotide further comprises a fourth domain (h) positioned between domain (f) and domain (e), wherein domain (h) comprises a replication blocker and wherein domain (h) is not part of domain (f).

8. The polynucleotide primer combination of claim 7, wherein domain (h) is part of domain (e).

9. The polynucleotide primer combination of claim 8, wherein domain (h) is at a position in domain (e) that is 1, 2, 3, 4 or 5 nucleotides from the 3′ end of domain (e).

10-38. (canceled)

39. The polynucleotide primer combination of claim 2 wherein the sequence of domain (f) is 100% complementary to the sequence of the second non-target polynucleotide region (F*).

40. (canceled)

41. The polynucleotide primer combination of claim 2 wherein the sequence of domain (f) comprises a mismatch with the sequence of the non-target polynucleotide region (F*).

42. The polynucleotide primer combination of claim 41 wherein the mismatch in the sequence of domain (f) with respect to the sequence of the non-target polynucleotide region (F*) is not a 3′ base mismatch.

43-45. (canceled)

46. The polynucleotide primer combination of claim 2 further comprising a blocker polynucleotide that comprises a 5′ terminus and a 3′ terminus.

47-55. (canceled)

56. The polynucleotide primer combination of claim 46 wherein the blocker polynucleotide is sufficiently complementary to hybridize under appropriate conditions to a third target polynucleotide region (X), wherein region (X) is between region (A) and region (F).

57. The polynucleotide primer combination of claim 56 wherein region (X) comprises a sequence that is at least about 1 nucleotide to about 100 kilobases in length.

58. The polynucleotide primer combination of claim 57 further comprising a first rigid frame blocker polynucleotide (RF1) and a second rigid frame blocker polynucleotide (RF2).

59. The polynucleotide primer combination of claim 58 wherein polynucleotide (RF1) comprises a domain (q) that is sufficiently complementary to a region (Xq) in a non-target polynucleotide to allow hybridization between domain (q) and region (Xq) under appropriate conditions, a domain (t) that is sufficiently complementary to a region (Xt) in the non-target polynucleotide to allow hybridization to allow hybridization between domain (t) and region (Xt), and a domain (r) that is sufficiently complementary to a domain (v) in polynucleotide (RF2) to allow hybridization between domain (r) and domain (v) under appropriate conditions; and
wherein polynucleotide (RF2) comprises a domain (u) that is sufficiently complementary to a region (Xu) in a non-target polynucleotide to allow hybridization between domain (u) and region (Xu) under appropriate conditions, a domain (w) that is sufficiently complementary to a region (Xw) in the non-target polynucleotide to allow hybridization between domain (w) and region (Xw), and domain (v) that is sufficiently complementary to domain (r) in polynucleotide (RF1) to allow hybridization between domain (r) and domain (v) under appropriate conditions, and wherein when domain (q) is specifically hybridized to region (Xq), and domain (t) is specifically hybridized to region (Xt), and domain (u) is specifically hybridized to region (Xu), and domain (w) is specifically hybridized to region (Xw), and domain (r) is specifically hybridized to domain (v), and domain (a) is specifically hybridized to region (A), domain (f) will not hybridize to region (F), and wherein region (Xq), region (Xt), region (Xu), region (Xu) and region (Xw) are not in the target polynucleotide.

60-66. (canceled)

67. The polynucleotide primer combination of claim 2 wherein region (A) is 5′ to region (F) in the target polynucleotide.

68. The polynucleotide primer combination of claim 67 wherein at least one nucleotide in domain (a) overlaps at least one nucleotide in domain (f).

69-73. (canceled)

74. A method of detecting a target polynucleotide in a sample with a primer combination, the primer combination comprising a first polynucleotide (1) and a second polynucleotide (2),
the first polynucleotide (1) comprising a first domain (a) having a sequence that is sufficiently complementary to a first target polynucleotide region (A), a second domain (c) comprising a unique polynucleotide sequence, and a third domain (b) comprising a sequence that is not sufficiently complementary to hybridize to a domain in the first polynucleotide (1), a domain in the second polynucleotide (2), or a domain in the target polynucleotide, or the third domain comprises a chemical polymer,
the second polynucleotide (2) comprising a first domain (f) that is fully complementary to a second target polynucleotide region (F), a second domain (d) comprising a polynucleotide sequence sufficiently complementary to domain (c) such that domain (c) and domain (d) will hybridize under appropriate conditions, and a third domain (e) comprising a sequence that is not sufficiently complementary to hybridize to a domain in the first polynucleotide (1), a domain in the second polynucleotide (2), or a domain in the target polynucleotide, domain (f) having a sequence that is not fully complementary to a non-target polynucleotide in the sample and
the method comprising the steps of:
contacting the sample with the primer combination and a polymerase under conditions that allow extension of a sequence from domain (f) which is complementary to the target polynucleotide when the second target polynucleotide region (F) is present in the sample and
detecting the sequence extended from domain (f) indicating the second target polynucleotide region (F) is present in the sample.

75. (canceled)

76. A method of detecting a target polynucleotide in a sample with a primer combination of claim 2 wherein the second polynucleotide (2) comprises a first domain that is fully complementary to region (F) and wherein domain (f) is not fully complementary to a non-target polynucleotide region in the sample,
the method comprising the steps of:
contacting the sample with the primer combination and a polymerase under conditions that allow extension of a sequence from domain (f) which is complementary to the target polynucleotide when the second target polynucleotide region (F) is present in the sample and
detecting the sequence extended from domain (f).

77-81. (canceled)

82. A method of initiating polymerase extension using a primer combination and a target polynucleotide as template in a sample, the primer combination comprising a first polynucleotide (1) and a second polynucleotide (2),
the first polynucleotide (1) comprising a first domain (a) having a sequence that is sufficiently complementary to a first target polynucleotide region (A), a second domain (c) comprising a unique polynucleotide sequence, and a third domain (b) comprising a sequence that is not sufficiently complementary to hybridize to a domain in the first polynucleotide (1), a domain in the second polynucleotide (2), or a domain in the target polynucleotide, or the third domain comprises a chemical polymer,
the second polynucleotide (2) comprising a first domain (f) that is fully complementary to a second target polynucleotide region (F), a second domain (d) comprising a polynucleotide sequence sufficiently complementary to domain (c) such that domain (c) and domain (d) will hybridize under appropriate conditions, and a third domain (e) comprising a sequence that is not sufficiently complementary to hybridize to a domain in the first polynucleotide (1), a domain in the second polynucleotide (2), or a domain in the target polynucleotide, domain (f) having a sequence that is not fully complementary to a non-target polynucleotide in the sample, and
wherein the sample comprises a mixture of (i) a target polynucleotide that has a sequence (F) that is fully complementary to the sequence in domain (f) and (ii) a non-target polynucleotide that has a sequence (F*) that is not fully complementary to (f), wherein the sequence of (F) is identical to the sequence of (F*) except for at least a one nucleotide difference,
the method comprising the step of contacting the sample with the primer combination and a polymerase under conditions that allow extension of a sequence from domain (f) and complementary to the target polynucleotide strand when domain (f) contacts region (F).

83-86. (canceled)

87. A method of initiating polymerase extension using the primer combination of claim 2 and a target polynucleotide as template in a sample, wherein the second polynucleotide (2) comprises a first domain (f) that is fully complementary to a first target polynucleotide region (F) and wherein domain (f) is not fully complementary to a non-target polynucleotide in the sample,
the method comprising the steps of:
contacting the sample with the primer combination and a polymerase under conditions that allow extension of a sequence from domain (f) which is complementary to the target polynucleotide when the target polynucleotide is present in the sample.

88-92. (canceled)

93. A method of amplifying a target polynucleotide in a sample using a polynucleotide primer combination, the primer combination comprising a first polynucleotide (1) and a second polynucleotide (2),
the first polynucleotide (1) comprising a first domain (a) having a sequence that is sufficiently complementary to a first target polynucleotide region (A), a second domain (c) comprising a unique polynucleotide sequence, and a third domain (b) comprising a sequence that is not sufficiently complementary to hybridize to a domain in the first polynucleotide (1), a domain in the second polynucleotide (2), or a domain in the target polynucleotide, or the third domain comprises a chemical polymer,
the second polynucleotide (2) comprising a first domain (f) that is fully complementary to a second target polynucleotide region (F), a second domain (d) comprising a polynucleotide sequence sufficiently complementary to domain (c) such that domain (c) and domain (d) will hybridize under appropriate conditions, and a third domain (e) comprising a sequence that is not sufficiently complementary to hybridize to a domain in the first polynucleotide (1), a domain in the second polynucleotide (2), or a domain in the target polynucleotide, domain (f) having a sequence that is not fully complementary to a non-target polynucleotide in the sample and
wherein the sample comprises a mixture of (i) a target polynucleotide that has a sequence in region (F) that is fully complementary to the sequence in domain (f) and (ii) one or more non-target polynucleotides that are not fully complementary to domain (f);
the method comprising the steps of:
(a) contacting the sample with the primer combination and a polymerase under conditions that allow extension of a sequence from domain (f) which is complementary to the target polynucleotide when the target polynucleotide is present in the sample,
(b) denaturing the sequence extended from domain (f) from the target polynucleotide, and
(c) repeating step (a) in the presence of a reverse primer having a sequence complementary to a region in the sequence extended from domain (f) in step (b) to amplify the target polynucleotide,
wherein extension and amplification of the target polynucleotide occurs when region (F) is fully complementary to the sequence in the domain (f) but is less efficient or does not occur when region (F) in the target polynucleotide is not fully complementary to the sequence in domain (f).

94. A method of amplifying a target polynucleotide in a sample using a polynucleotide primer combination of claim 2, wherein the second polynucleotide (2) comprises a first domain (f) that is fully complementary to a first target polynucleotide region (F) and wherein domain (f) is not fully complementary to a non-target polynucleotide in the sample,
the method comprising the steps of:
(a) contacting the sample with the primer combination and a polymerase under conditions that allow extension of a sequence from domain (f) which is complementary to the target polynucleotide when the target polynucleotide is present in the sample,
(b) denaturing the sequence extended from domain (f) from the target polynucleotide, and
(c) repeating step (a) in the presence of a reverse primer having a sequence complementary to a region in the sequence extended from (f) in step (b) to amplify the target polynucleotide,
wherein extension and amplification of the target polynucleotide occurs when region (F) is fully complementary to the sequence in domain (f) but is less efficient or does not occur when the first region in the target polynucleotide is not fully complementary to the sequence in domain (f).

95-101. (canceled)
